(19) United States
(12) Patent Application Publication
(10) Pub. No.: US 2026/0285801 A1
(43) Pub. Date: Sep. 24, 2026

(54) LIPID NANOPARTICLE FOR TARGETED DELIVERY OF THERAPEUTIC PAYLOADS

(71) Applicant: Trustees of Tufts College, Medford, MA (US)

(72) Inventors: Qiaobing Xu, Medford, MA (US); Haishan Li, Winchester, MA (US); Changfeng Huang, Arlington, MA (US)

(21) Appl. No.: 19/164,106

(22) PCT Filed: Mar. 13, 2024
(86) PCT No.: PCT/US2024/019723

§ 371 Date: Sep. 10, 2025

Related U.S. Application Data
(60) Provisional application No. 63/489,983, filed on Mar. 13, 2023.

(51) Int. Cl. C07C 229/16; A61K 9/51; A61K 31/7105; C07C 271/12; C07C 271/20; C07C 275/14; C07C 323/12; C07C 333/04; C07C 335/08; C07C 391/00; C07D 211/26; C07D 233/61; C07D 295/13

(52) U.S. Cl. CPC C07C 229/16; A61K 9/5123; A61K 31/7105; C07C 271/12; C07C 271/20; C07C 275/14; C07C 323/12; C07C 333/04; C07C 335/08; C07C 391/00; C07D 211/26; C07D 233/61; C07D 295/13; C07C 2601/02

(57) ABSTRACT

Disclosed are compounds, compositions, and methods related to ionizable lipids and lipid nanoparticles (LNPs) comprising the ionizable lipids. The LNPs can comprise a therapeutic peptide or a nucleic acid molecule encoding the therapeutic peptide for immunotherapy.

### CROSS-REFERENCE TO RELATED APPLICATIONS

This application claims the benefit of priority to U.S. Provisional Application No. 63/489,983, filed Mar. 13, 2023; the contents of which are fully incorporated herein by reference.

### BACKGROUND

Synthetic mRNA provides a template for the synthesis of any given peptide, protein or protein fragment and lends itself to a broad range of pharmaceutical applications, including different modalities of cancer immunotherapy. To function in vivo, mRNA requires safe, effective, and stable delivery systems that protect the nucleic acid from degradation and that allow cellular uptake and mRNA release. Lipid nanoparticles have successfully entered the clinic for the delivery of mRNA; in particular, lipid nanoparticle-mRNA vaccines have been approved in clinical use against coronavirus disease 2019 (COVID-19), which marks a milestone for mRNA therapeutics. Many other lipid nanoparticle-mRNA formulations have been developed and are under clinical evaluation for the prevention and treatment of viral infections, cancer, and genetic diseases.

### SUMMARY

In some aspects, the present disclosure provides a compound represented by Formula (III):

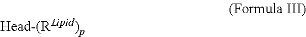

or a pharmaceutically acceptable salt thereof;
wherein:
the Head is selected from

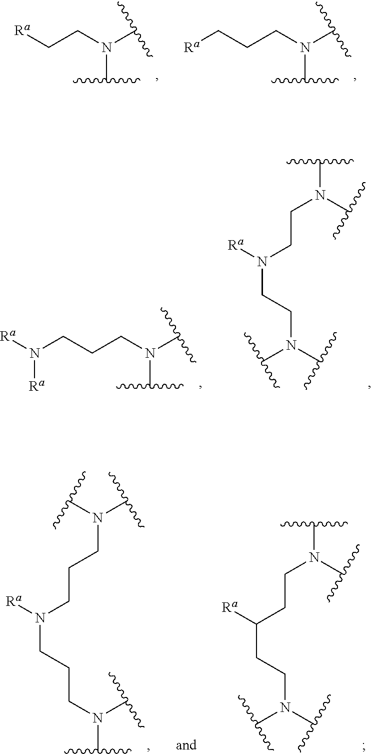

wherein each R^{a} is independently selected from H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₃-C₇ carbocycle, and 3- to 7-membered heterocycle;
each R^{Lipid} is independently selected from:

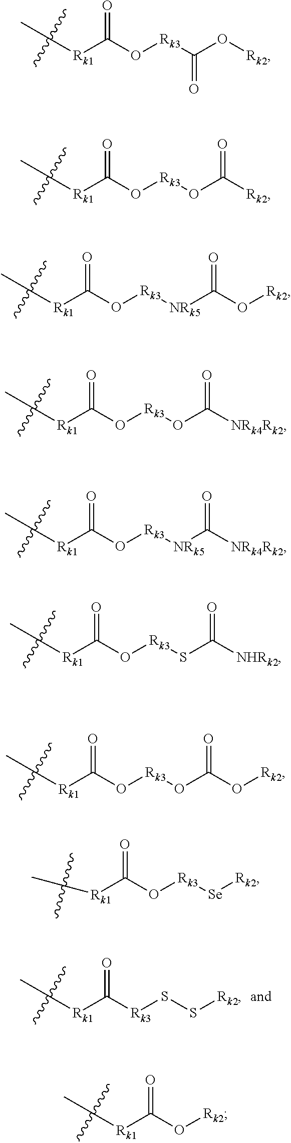

each Rₖ₁ and Rₖ₃ are independently selected from C₁-C₁₀ alkyl;
each Rₖ₂ is independently selected from C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl;
each Rₖ₄ and Rₖ₅ are independently selected from H and C₁-C₁₀ alkyl; and
p is selected from 1, 2, 3, 4, 5, and 6.

In some embodiments, R^{Lipid} is selected from the group consisting of

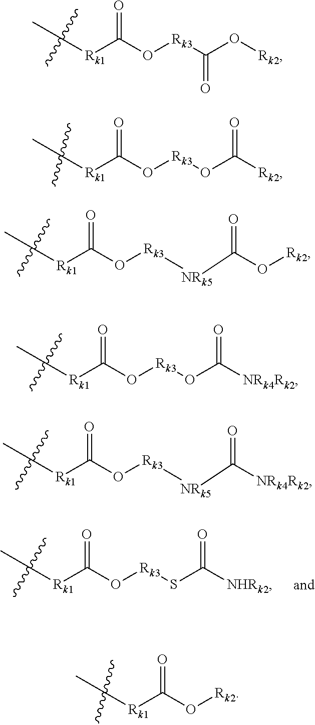

In some embodiments, the Head is selected from the group consisting of

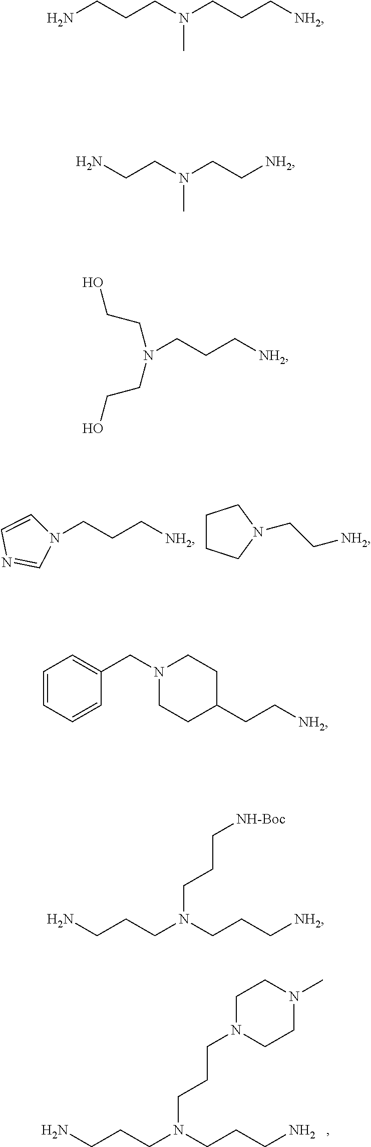

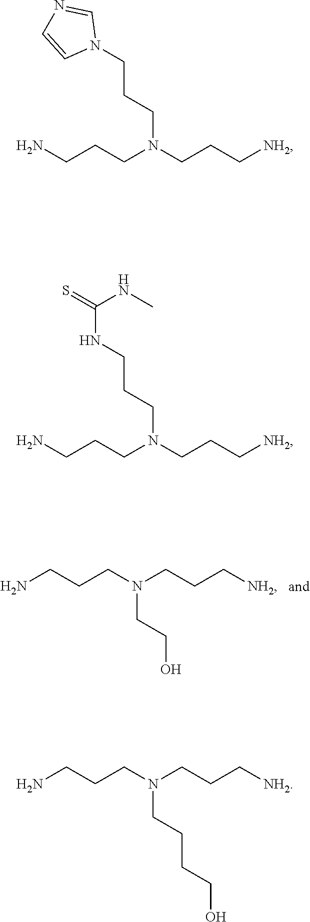 [SMILES: NCCCN(CCCN)CCCn1ccnc1 ; CNC(=S)NCCCN(CCCN)CCCN ; NCCCN(CCO)CCCN ; "and" ; NCCCN(CCCN)CCCCO]

In some embodiments, R^{Lipid} is represented by a structure in TABLE 1. In some embodiments, the compound is represented by a structure in TABLE 2.

In some aspects, the present disclosure provides a composition comprising a pharmaceutical agent assembled with a lipid composition comprising an ionizable lipid represented by Formula (III):

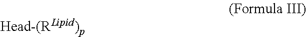

or a pharmaceutically acceptable salt thereof;
wherein:
the Head is selected from

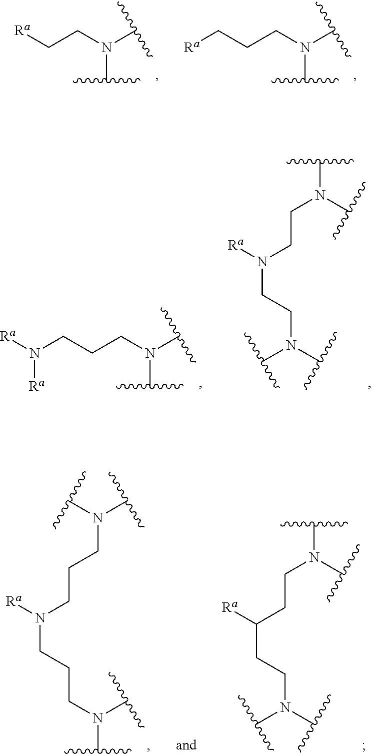

wherein each R^{a} is independently selected from H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₃-C₇ carbocycle, and 3- to 7-membered heterocycle;
each R^{Lipid} is independently selected from:

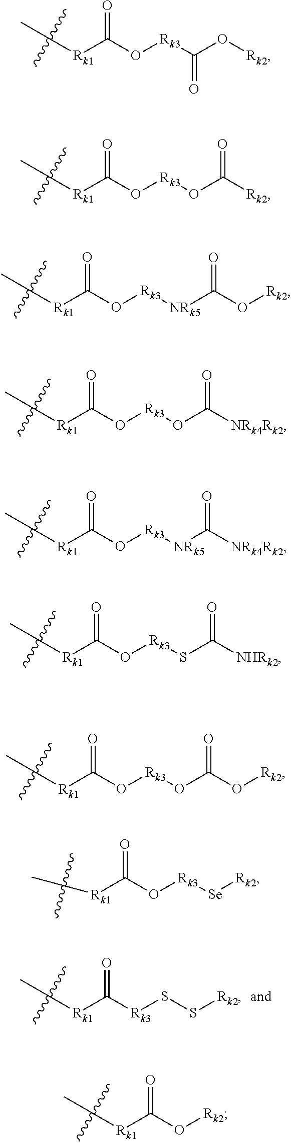

each of Rₖ₁ and Rₖ₃ are independently selected from C₁-C₁₀ alkyl;
each Rₖ₂ is independently selected from C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl;
each Rₖ₄ and Rₖ₅ are independently selected from H and C₁-C₁₀ alkyl; and
p is selected from 1, 2, 3, 4, 5, and 6.

In some aspects, the present disclosure provides a composition, comprising a pharmaceutical agent assembled with a lipid composition that comprises an ionizable lipid, wherein the ionizable lipid has an amine head group and at least one hydrophobic tail R^{Lipid} having a structure of Formula (I):

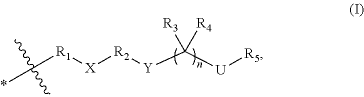

or a pharmaceutically acceptable salt thereof;
wherein:
* indicates a point of attachment to a nitrogen in the amine headgroup;
R₁ and R₂ are each independently selected from C₁-C₁₂ alkylene, C₁-C₁₂ alkenylene, and 1- to 12-membered heteroalkylene;
X is

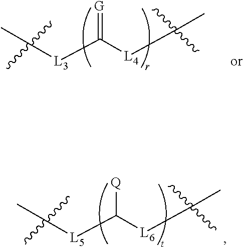

in which each of L₃, L₄, L₅, and L₆ is, independently, a bond, O, S, or NR^{c}; G is O, S, or NR^{d}; Q is OR^{e}, SR^{f}, or NR^{g}R^{h}; r is selected from 1, 2, 3, 4, 5, and 6; and t is t is selected from 1, 2, 3, 4, 5, and 6;
each of R^{c}, R^{d}, R^{e}, R^{f}, R^{g}, and R^{h} is independently H, C1-C10 alkyl, 1- to 10-membered heteroalkyl, aryl, or heteroaryl;
Y and U are each independently a bond, O, S, NR¹⁰, or Se;
n is 0 or 1;
R₃ and R₄ are each independently H, C1-C10 alkyl, 1- to 10-membered heteroalkyl, aryl, or heteroaryl; or R₃ and R₄ together with the atom to which they are attached, form ═O;
R₅ is a C1-C20 alkyl, C2-C20 alkenyl, C2-C20 alkynyl, C3-C20 cycloalkyl, 1- to 20-membered heteroalkyl, 3- to 20-membered heterocycloalkyl, aryl, or heteroaryl; and
the pharmaceutical agent comprises an mRNA encoding at least one therapeutic peptide for immunotherapy.

In some embodiments, the amine head group is represented by

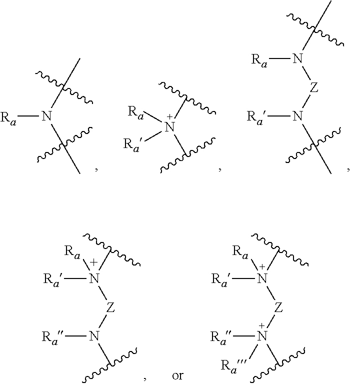

wherein Ra, Ra′, Ra″, and Ra′″ are each independently, H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₆-C₂₀ aryl, and 6- to 20-membered heteroaryl, or a R^{Lipid}

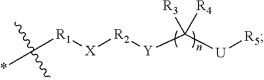

and Z is selected from C₁-C₂₀ alkylene, a 1- to 20-membered heteroalkylene, arylene, and heteroarylene.

In some embodiments, the ionizable lipid is represented by Formula (II):

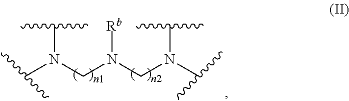

or a pharmaceutically acceptable salt thereof, wherein:
i) R^{b} is a substituted or unsubstituted C₁-C₆ alkyl, hydroxyalkyl, alkoxyalkyl, or aryl; and
ii) n1 and n2 are each independently 1, 2, 3, 4, 5, or 6.

In some embodiments, the amine head group is selected from the group consisting of

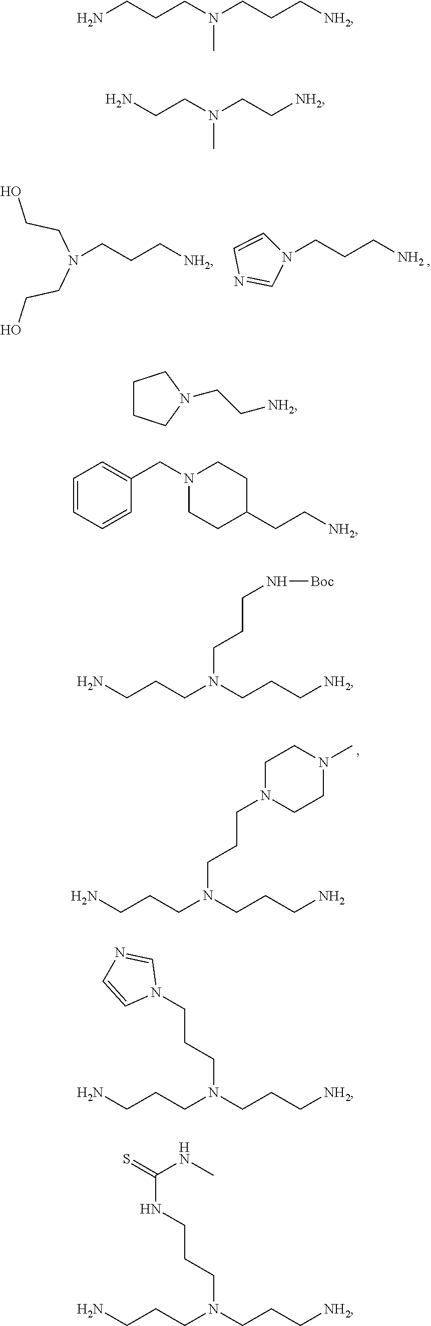

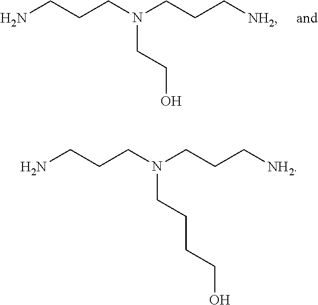 [SMILES: NCCCN(CCO)CCCN ; NCCCN(CCCN)CCCCO]

In some embodiments, the hydrophobic tail R^{Lipid} has a structure of

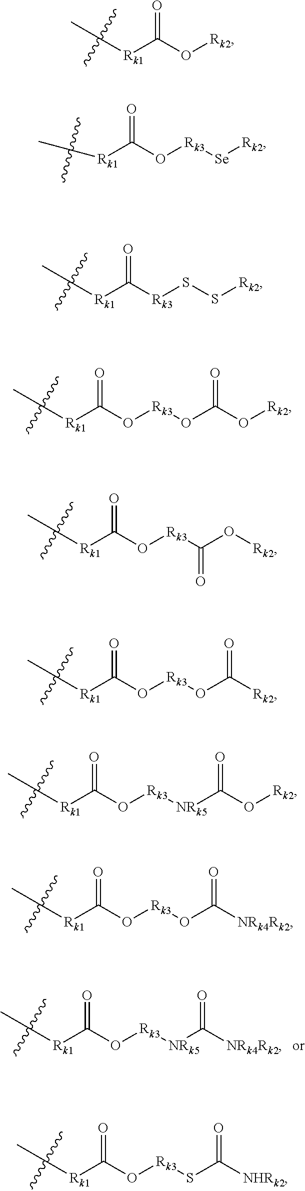

wherein
each Rₖ₁ and Rₖ₃ is independently selected from C₁-C₁₀ alkyl;
each Rₖ₂ is independently selected from C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl; and
each Rₖ₄ and Rₖ₅ is independently selected from H and C₁-C₁₀ alkyl.

In some embodiments, the lipid composition further comprises a steroid. In some embodiments, the steroid is cholesterol or a cholesterol derivative. In some embodiments, the lipid composition further comprises a helper lipid. In some embodiments, the helper lipid is 1,2-dioleoyl-sn-glycero-3-phosphoethanolamine (DOPE) or 1,2-dioleoyl-sn-glycero-3-phosphocholine (DOPC). In some embodiments, the lipid composition further comprises a polymer conjugated lipid. In some embodiments, the polymer conjugated lipid is a PEG conjugated lipid. In some embodiments, the polymer conjugated lipid is 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k). In some embodiments, the lipid composition comprises a transactivator of transcription (TAT) peptide modification. In some embodiments, the lipid composition further comprises a steroid, a helper lipid, and a polymer conjugated lipid. In some embodiments, the ionizable lipid is present in the lipid composition at a weight percentage from about 30% to about 90%. In some embodiments, the steroid is present in the lipid composition at a weight percentage from about 10% to about 40%. In some embodiments, the helper lipid is present in the lipid composition at a weight percentage from about 1% to about 20%. In some embodiments, the polymer conjugated lipid is present in the lipid composition at a weight percentage from about 1% to about 20%. In some embodiments, the weight ratio of the ionizable lipid/steroid/helper lipid/polymer conjugated lipid is about 16/4/1/1. In some embodiments, the weight ratio of the ionizable lipid/steroid/helper lipid/polymer conjugated lipid is about 16.8/4/2/1. In some embodiments, the weight ratio of the ionizable lipid/steroid/helper lipid/polymer conjugated lipid is about 16.6/4/2/1. In some embodiments, the weight ratio of the pharmaceutical agent/lipid composition is from about 1:200 to about 1:5.

In some embodiments, the lipid composition further comprises a steroid and a helper lipid. In some embodiments, the ionizable lipid is present in the lipid composition at a weight percentage from about 30% to about 90%. In some embodiments, the helper lipid is present in the lipid composition at a weight percentage from about 5% to about 40%. In some embodiments, the steroid is present in the lipid composition at a weight percentage from about 5% to about 40%. In some embodiments, the weight ratio of the ionizable lipid/steroid/helper lipid is about 2/1/1.

In some embodiments, the lipid composition further comprises an excipient. In some embodiments, the excipient is selected from the group consisting of (2-hydroxypropyl)-β-cyclodextrin ((HP-β-CD), stearic acid, Perfluoroundecanoic, Saponin, Mannitol, Borneol, Amikacin-EC16, Kanamycin-EC16, Neomycin-EC16, 80-EC16, and Bile salts. In some embodiments, the excipient is present in the composition at a weight percentage from about 5% to about 60%. In some embodiments, the pharmaceutical agent comprises a polynucleotide, an oligonucleotide, a polypeptide, an oligopeptide, a small molecule compound, or any combination thereof. In some embodiments, the pharmaceutical agent comprises: (a) a gene modulating moiety configured to specifically bind at least a portion of a target gene or a gene product thereof, or (b) a polynucleotide that encodes a gene modulating moiety configured to specifically bind at least a portion of a target gene or a gene product thereof. In some embodiments, the gene modulating moiety comprises a guide nucleic acid configured to complex with at least a portion of the target gene or the gene product thereof, or a polynucleotide sequence that encodes the guide nucleic acid. In some embodiments, the gene modulating moiety comprises a heterologous endonuclease or a polynucleotide comprising a sequence that encodes the heterologous endonuclease. In some embodiments, the ionizable lipid comprises at least two hydrophobic tails, wherein not all hydrophobic tails are identical.

In some embodiments, the ionizable lipid comprises at least two hydrophobic tails, wherein two or more hydrophobic tails are identical.

In some aspects, the present disclosure provides a method for delivering a pharmaceutical agent to a target organ in a subject in need thereof, the method comprising administering an effective amount of any composition disclosed in this application.

In some aspects, the present disclosure provides a method for delivering a pharmaceutical agent to a target organ in a subject in need thereof, the method comprising: administering to the subject an effective amount of the pharmaceutical agent assembled with a lipid composition disclosed herein that comprises an ionizable lipid, a steroid, a helper lipid, and a polymer conjugated lipid; and thereby providing a greater amount or activity of the pharmaceutical agent in the target organ in the subject as compared to that achieved absent the lipid composition.

In some aspects, the present disclosure provides a method for delivering a pharmaceutical agent to a target organ in a subject in need thereof, the method comprising administering to the subject an effective amount of the pharmaceutical agent assembled with a lipid composition according disclosed herein that comprises an ionizable lipid, a steroid, a helper lipid, and a polymer conjugated lipid; and thereby providing a greater amount or activity of the pharmaceutical agent in the target organ in the subject as compared to a non-target organ. In some embodiments, the administering is through systemic administration. In some embodiments, the administering is through intramuscular administration. In some embodiments, the administering is through intravenous administration. In some embodiments, the pharmaceutical agent is delivered at a dosage of no more than 3 mg/kg body weight. In some embodiments, the pharmaceutical agent is delivered at a dosage from about 1 μg/kg body weight to about 3 mg/kg body weight. In some embodiments, the pharmaceutical agent is delivered in one or more doses. In some embodiments, the pharmaceutical agent is delivered for cancer immunotherapy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B illustrate synthesis schemes of lipid tails. FIG. 1A depicts the synthesis of lipid tail 012B. FIG. 1B depicts the synthesis of lipid tail Compound 4 comprising a carbonate.

FIGS. 2A-2G show whole body bioluminescence from IVIS imaging of adult BALB/c mice at about 6 hours post single i.v. injections of luciferase mRNA/LNPs.

FIGS. 3A-3C depict plasma concentrations of hEPO in CD1 mice at 6 hours post single IM injections of hEPO mRNA/LNPs.

FIGS. 4A-4B show whole body bioluminescence from IVIS imaging of adult BALB/c mice at about 6 hours post single i.v. injections of luciferase mRNA/LNPs. Lipid nanoparticles were formulated with luciferase mRNA and lipids L88, L854, and L855, respectively. IVIS images and structures of the three lipids are shown in FIG. 4A. Quantification of luciferase intensity is shown in FIG. 4B. The experiment was conducted in duplicates. However, due to lab error the second mouse intended for testing the luciferase mRNA/L854-LNP did not receive the full injection.

FIGS. 5A-5B show whole body bioluminescence from IVIS imaging of adult BALB/c mice at about 6 hours post single i.v. injections of luciferase mRNA/LNPs. Lipid nanoparticles were formulated with luciferase mRNA and lipids L88, L854, and L855, respectively. IVIS images are shown in FIG. 5A. Quantification of luciferase intensity is shown in FIG. 5B. The experiment was conducted in duplicates.

FIGS. 6A-6B show whole body bioluminescence from IVIS imaging of adult BALB/c mice at about 6 hours post single i.v. injections of luciferase mRNA/LNPs. Lipid nanoparticles were formulated with luciferase mRNA and lipids L854 and ALC-0315, respectively. IVIS images are shown in FIG. 6A. Quantification of luciferase intensity is shown in FIG. 6B. The experiment was conducted in duplicates.

FIGS. 7A-7B show whole body bioluminescence from IVIS imaging of adult BALB/c mice at about 6 hours post single i.v. injections of luciferase mRNA/LNPs. Lipid nanoparticles were formulated with luciferase mRNA and lipids L855 and SM-102, respectively. IVIS images are shown in FIG. 7A. Quantification of luciferase intensity is shown in FIG. 7B. The experiment was conducted in duplicates.

### DETAILED DESCRIPTION

The present disclosure provides ionizable lipids and compositions, methods, and kits related to lipid nanoparticles (LNPs) formulated by the ionizable lipids and a therapeutic payload. In some embodiments, the therapeutic payload comprises a therapeutic peptide or a nucleic acid molecule encoding the therapeutic peptide. In some embodiments, the LNPs comprise an mRNA that encodes a therapeutic peptide for immunotherapy. In some embodiments, the therapeutic peptide for immunotherapy comprises an antigen-binding peptide, an antigen receptor, or an immunomodulators. In some embodiments, the LNPs can deliver the therapeutic payload to a target organ or cell. In some embodiments, the target organ comprises a liver or a spleen.

### Definitions

Before the embodiments of the disclosure are described, it is to be understood that such embodiments are provided by way of example only, and that various alternatives to the embodiments of the disclosure described herein may be employed in practicing the disclosure. Numerous variations, changes, and substitutions will now occur to those skilled in the art without departing from the disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the disclosure.

In the context of the present application, the following terms have the meanings ascribed to them unless specified otherwise:

As used throughout the specification and claims, the terms “a”, “an” and “the” are generally used in the sense that they mean “at least one”, “at least a first”, “one or more” or “a plurality” of the referenced components or steps, except in instances wherein an upper limit is thereafter specifically stated. For example, a “cleavage sequence”, as used herein, means “at least a first cleavage sequence” but includes a plurality of cleavage sequences. The operable limits and parameters of combinations, as with the amounts of any single agent, will be known to those of ordinary skill in the art in light of the present application.

The terms “polypeptide”, “peptide”, and “protein” are used interchangeably herein to generally refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein, the term “antibody” refers to an immunoglobulin (Ig) whether natural or partly or wholly synthetically produced. The term also covers any polypeptide or protein having a binding domain which is, or is homologous to, an antigen-binding domain. The term further includes “antigen-binding fragments” or “functional fragment thereof”, or “fragment of an antibody”, “antibody fragment”, “functional fragment of an antibody” and other interchangeable terms for similar binding fragments such as described below. An antibody includes, for example, monoclonal antibodies, chimeric antibodies, humanized antibodies, human antibodies, recombinant antibodies, chemically engineered antibodies, deimmunized antibodies, affinity-matured antibodies, multispecific antibodies (for example, bispecific antibodies and polyreactive antibodies), heteroconjugate antibodies, antibody fragments, and combinations thereof (e.g., a monoclonal antibody that is also deimmunized, a humanized antibody that is also deimmunized, etc.). An antibody can be, for example, murine, chimeric, humanized, heteroconjugate, bispecific, diabody, triabody, or tetrabody. The antigen binding fragment can include, for example, Fab′, F(ab′)2, Fab, Fv, rlgG, scFv, hcAbs (heavy chain antibodies), a single domain antibody, VHH, VNAR, sdAbs, or nanobody.

The term “antigen” refers to a molecule bound by an antibody or a fragment thereof. Antigens may be proteins recognized by immunoglobulins, in which case the sites on the proteins bound by the immunoglobulins are referred to as “epitopes”. Antigens may also, or alternatively, be recognized by non-immunoglobulin ligand binding domain of an antigen-binding peptide. In such cases, “antigen” refers to binding partners of the non-immunoglobulin ligand binding domain of the bispecific binding peptide. In some cases, the antigen can be a ligand of a cell surface receptor, e.g., a ligand of NKG2D. In some cases, the bispecific antigen-binding peptide contains a cell surface receptor ligand, or receptor binding portion thereof, and the antigen is the ligand binding portion of the cell surface receptor.

As used herein, the terms “treatment” or “treating,” or “palliating” or “ameliorating” are used interchangeably herein. These terms generally refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms or improvement in one or more clinical parameters associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect” or “therapeutic benefit,” as used herein, generally refers to a physiologic effect, including but not limited to the mitigation, amelioration, or prevention of disease or an improvement in one or more clinical parameters associated with the underlying disorder in humans or other animals, or to otherwise enhance physical or mental wellbeing of humans or animals, resulting from administration of a polypeptide of the disclosure other than the ability to induce the production of an antibody against an antigenic epitope possessed by the biologically active protein. For prophylactic benefit, the compositions may be administered to a subject at risk of developing a particular disease, a recurrence of a former disease, condition or symptom of the disease, or to a subject reporting one or more of the physiological symptoms of a disease, even though a diagnosis of this disease may not have been made.

The terms “therapeutically effective amount” and “therapeutically effective dose”, as used herein, generally refer to an amount of a drug or a biologically active protein, either alone or as a part of a polypeptide composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl(C≤8)” or the class “alkene(C≤8)” is two. Compare with “alkoxy(C≤10)”, which designates alkoxy groups having from 1 to 10 carbon atoms. “Cm-n” or “Cm-Cn” defines both the minimum (m) and maximum number (n) of carbon atoms in the group. Thus, “C1-C10 alkyl” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefincs” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” generally signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃ (tert-butyl, t-butyl, t-Bu or ⁱBu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H-R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, the carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

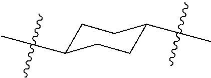 [SMILES: *C1CCC(*)CC1]

is a non-limiting example of cycloalkanediyl group. A “cycloalkane” refers to the class of compounds having the formula H-R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H-R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C—CH, —C—CCH₃, and —CH₂C—CCH₃ are non-limiting examples of alkynyl groups. An “alkyne” refers to the class of compounds having the formula H-R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, the carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, the carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

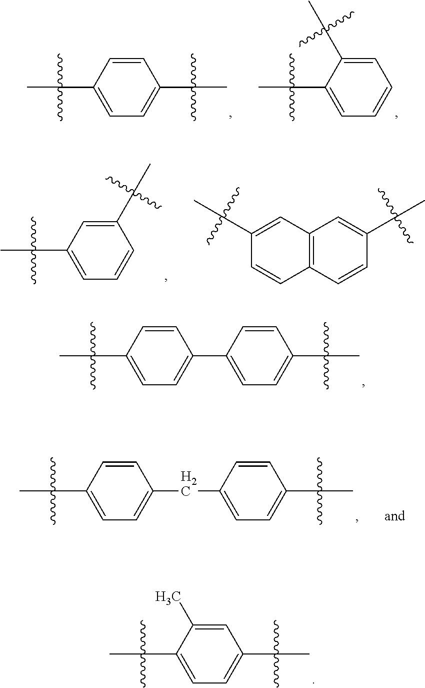

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

“Carbocycle” refers to a saturated, unsaturated or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Bicyclic carbocycles may be fused, bridged or spiro-ring systems. In some cases, spiro-ring carbocycles have at least two molecular rings with only one common atom.

“Heterocycle” refers to a saturated or unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. Bicyclic heterocycles may be fused, bridged or spiro-ring systems. In some cases, spiro-ring heterocycles have at least two molecular rings with only one common atom. The spiro-ring heterocycle includes at least one heteroatom.

“Heterocyclene” refers to a divalent heterocycle linking the rest of the molecule to a radical group.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” or “C₁-C₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆ alkylene- may be selected from methylene, ethylene, propylene, butylene, pentylene, and hexylene, any one of which is optionally substituted.

The term “hetero” when used to modify a compound or chemical group means the compound or chemical group has at least an atom that is not carbon, for example, N, O, S, Se, P, Si, B, or any other heteroatom. For example, a heteroaliphatic can be any aliphatic moiety containing at least one heteroatom selected from N, O, P, B, S, Si, Sb, Al, Sn, As, Se, and Ge. A heterocycle can be any ring containing a ring atom that is not carbon. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

The term “heteroaryl” when used without the “substituted” modifier refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

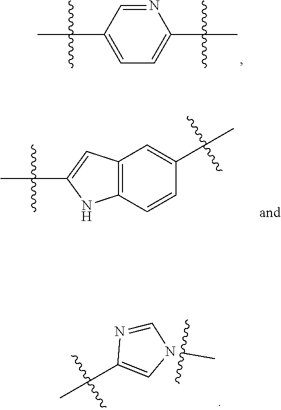

The term “heterocycloalkyl” when used without the “substituted” modifier refers to a monovalent non-aromatic group with a carbon atom or nitrogen atom as the point of attachment, the carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to a divalent cyclic group, with two carbon atoms, two nitrogen atoms, or one carbon atom and one nitrogen atom as the two points of attachment, the atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

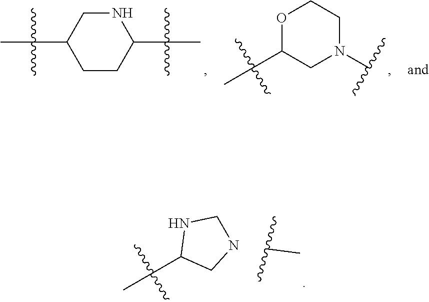

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃.

The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. Unless specified otherwise, aliphatic, heteroaliphatic, oxyaliphatic, alkyl, alkylene, alkenyl, alkenylene, alkynyl, alkynylene, cycloalkyl, cycloalkylene, cycloalkenyl, cycloalkenylene, cycloalkynyl, cycloalkynylene, hydroxyalkyl, heterocycloalkyl, heterocycloalkylene, heterocycloalkenyl, heterocycloalkenylene, aryl, and heteroaryl mentioned herein include both substituted and unsubstituted moieties.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally, the term “about,” as used herein when referring to a measurable value such as an amount of weight, time, dose, etc. is meant to encompass in one example variations of ±20% or ±10%, in another example ±5%, in another example ±3%, in another example ±1%, and in yet another example ±0.1% from the specified amount, as such variations are appropriate to perform the disclosed method.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to affect such treatment for the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate (e.g., non-human primate). In certain embodiments, the patient or subject is a human. Non-limiting examples of human subjects are adults, juveniles, infants, and fetuses.

The term “assemble” or “assembled,” as used herein, in context of delivery of a payload to target cell(s) generally refers to covalent or non-covalent interaction(s) or association(s), for example, such that a therapeutic or prophylactic agent be complexed with or encapsulated in a lipid composition.

As used herein, the term “lipid composition” generally refers to a composition comprising lipid compound(s), including but not limited to, a lipoplex, a liposome, a lipid particle. Examples of lipid compositions include suspensions, emulsions, and vesicular compositions.

A “payload”, as used herein, refers to an agent encapsulated within the lipid compositions or lipid nanoparticles (LNPs) provided herein. A payload can comprise a therapeutic payload, such as an active pharmaceutical ingredient. For example, a therapeutic payload can be a biologic drug (e.g., a peptide or a nucleic acid molecule) to be encapsulated within the LNPs. A therapeutic payload can also be a small molecule drug, such as a chemotherapeutic agent. In some embodiments, a plurality of payloads can be encapsulated within the LNPs.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present application which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically acceptable material, composition, or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The term “helper lipid” as used in this disclosure refers to a lipid that contributes to the stability or delivery efficacy of a lipid composition. A helper lipid can be a zwitterionic lipid, such as a phospholipid. A helper lipid can be phosphatidylcholine, distearoylphosphatidylcholine, dioleoylphosphatidylethanolamine, 1,2-dioleoyl-sn-glycero-3-phosphoethanolamine (DOPE) or 1,2-dioleoyl-sn-glycero-3-phosphocholine (DOPC). In some cases, the term “helper lipid” refers to phospholipids or other zwitterionic lipids in the LNP composition. In some cases, when describing the formulation of an LNP using weight ratios of the lipid components (e.g., lipidoid, steroid, helper lipid, and polymer conjugated lipid), a helper lipid refers to a phospholipid or another zwitterionic lipid. For example, the weight ratio of the lipidoid/steroid/helper lipid/polymer conjugated lipid is about 14/4/1/1. “Helper lipid” can refer to any class of lipid molecules that improves the particle stability and fluidity of lipid nanoparticles (LNP). Several classes of molecules can be used as helper lipids such as phospholipids (e.g., phosphoethanolamine, phosphocholine), zwitterionic lipids, steroid derivatives, and polymer conjugated lipids (e.g., PEGylated lipid). Representative helper lipids include cholesterol, 1,2-dioleoyl-sn-glycero-3-phosphoethanolamine (DOPE), 1,2-dioleoyl-sn-glycero-3-phosphocholine (DOPC), Phosphatidylcholine (PC), Methoxy-Polyethyleneglycol (MW 2k)-distearoylphosphatidylethanolamine (mPEG2k-DSPE), and 1,2-dimyristoyl-rac-glycero-3-methoxypolyethylene glycol-2000 (DMG-PEG2k).

### Lipids and Compositions

In some aspects, provided herein is a compound represented by Formula (III):

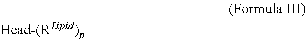

or a pharmaceutically acceptable salt thereof;
wherein:
the Head is selected from

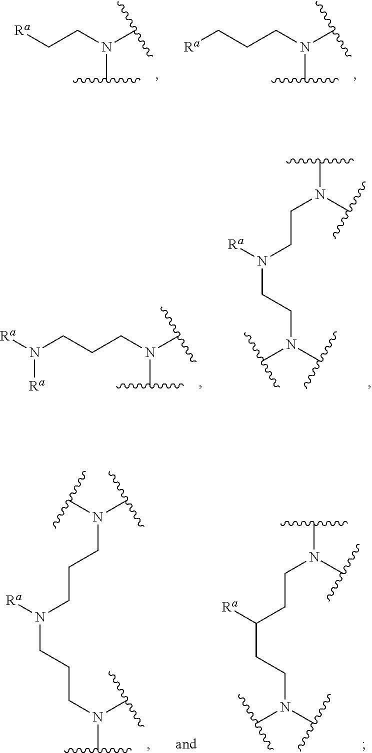

wherein each R^{a} is independently selected from H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₃-C₇ carbocycle, and 3- to 7-membered heterocycle;
each R^{Lipid} is independently selected from:

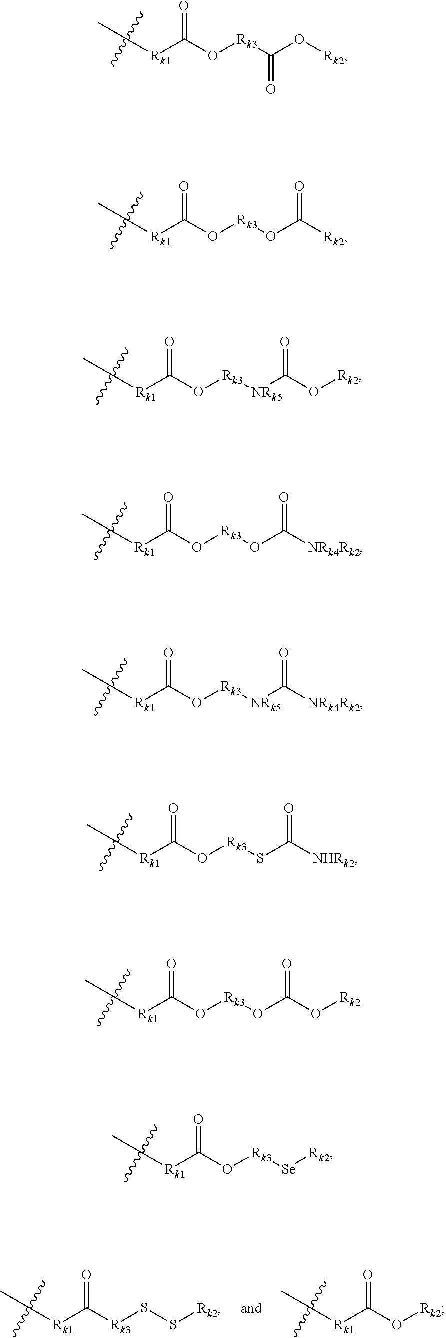

each Rₖ₁ and Rₖ₃ are independently selected from C₁-C₁₀ alkyl;
each Rₖ₂ is independently selected from C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl;
each Rₖ₄ and Rₖ₅ are independently selected from H and C₁-C₁₀ alkyl; and
p is selected from 1, 2, 3, 4, 5, and 6.

In some embodiments of a compound or salt of Formula (III), the Head is selected from

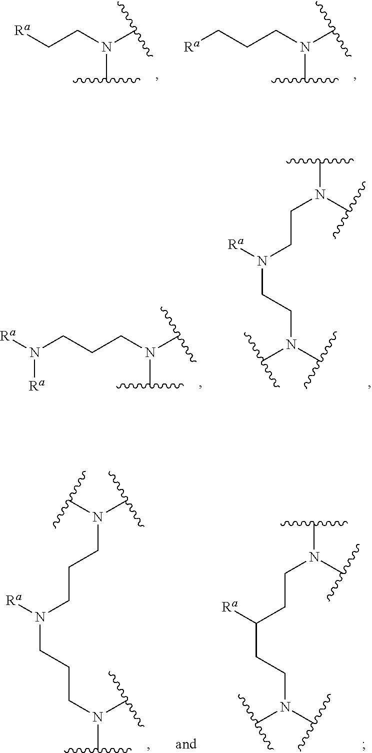

wherein each R^{a} is independently selected from H, C₁-C₁₀ alkyl, C₂-C₁₀ alkenyl, C₂-C₁₀ alkynyl, C₃-C₁₀ cycloalkyl, 3- to 10-membered heterocycloalkyl, 1- to 10-membered heteroalkyl, C₃-C₇ carbocycle, and 3- to 7-membered heterocycle. In some embodiments, each R^{a} is independently selected from H, C₁-C₅ alkyl, C₂-C₁₀ alkenyl, C₂-C₁₀ alkynyl, C₃-C₁₀ cycloalkyl, 3- to 10-membered heterocycloalkyl, 1- to 10-membered heteroalkyl, C₃-C₇ carbocycle, and 3- to 7-membered heterocycle. In some embodiments, each R^{a} is independently selected from H, C₁-C₅ alkyl, C₃-C₁₀ cycloalkyl, 3- to 10-membered heterocycloalkyl, 1- to 10-membered heteroalkyl, C₃-C₇ carbocycle, and 3- to 7-membered heterocycle.

In some embodiments of a compound or salt of Formula (III), each R^{a} is independently selected from H, methyl,

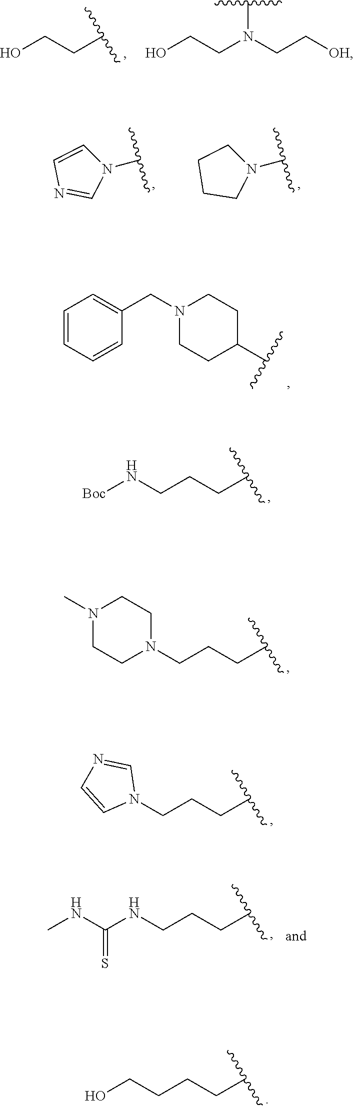

In some embodiments, each R^{a} is independently selected from H, methyl,

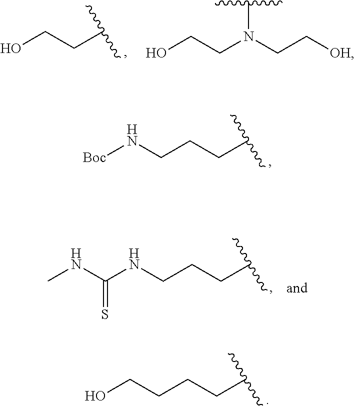

In some embodiments, each R^{a} is independently selected from

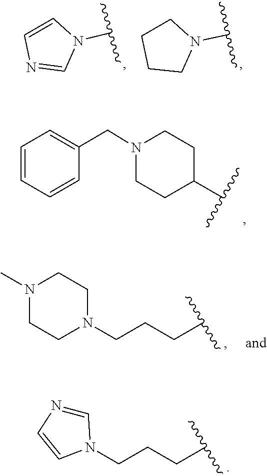

In some embodiments of a compound or salt of Formula (III), the Head is selected from

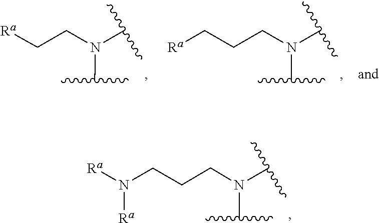

wherein each R^{a} is independently selected from H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₃-C₇ carbocycle, and 3- to 7-membered heterocycle. In some embodiments, Head is selected from

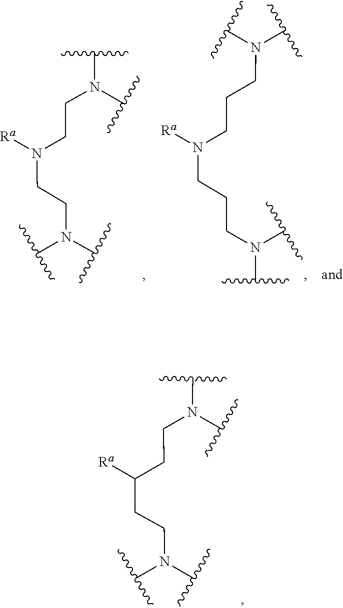

wherein each R^{a} is independently selected from H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₃-C₇ carbocycle, and 3- to 7-membered heterocycle

In some embodiments of a compound or salt of Formula (III) or (I), R^{Lipid} is selected from the group consisting of

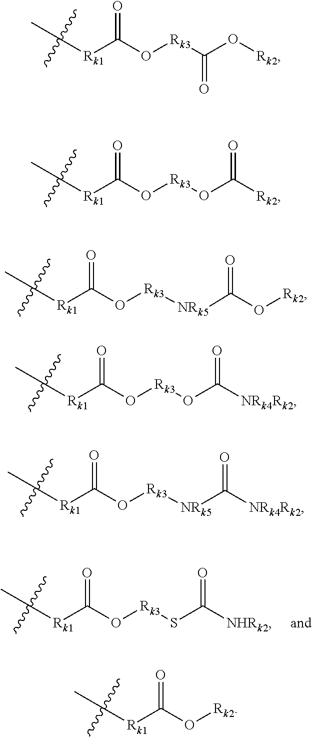

In some embodiments, R^{Lipid} is selected from the group consisting of

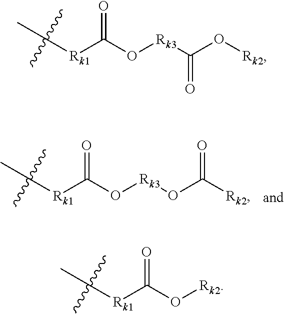

In some embodiments R^{Lipid} is selected from the group consisting of

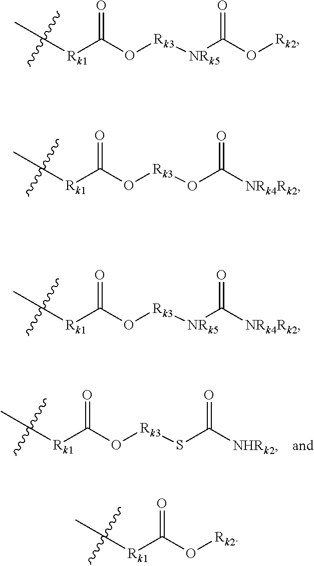

In some embodiments of a compound or salt of Formula (III), the Head is selected from the group consisting of

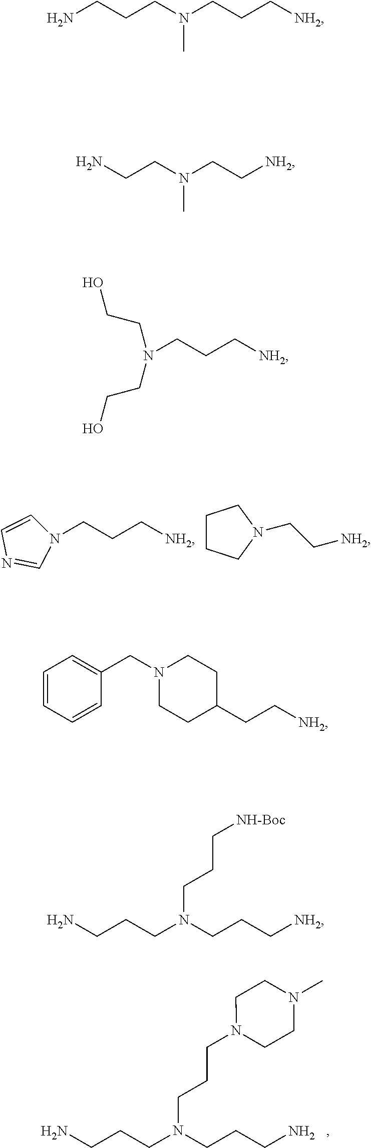

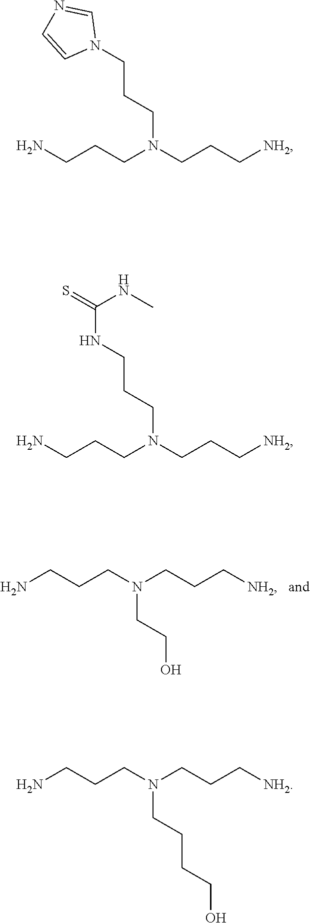 [SMILES: NCCCN(CCCN)CCCn1ccnc1 ; CNC(=S)NCCCN(CCCN)CCCN ; NCCCN(CCO)CCCN ; "and" ; NCCCN(CCCN)CCCCO]

In some embodiments, the Head is selected from the group consisting of

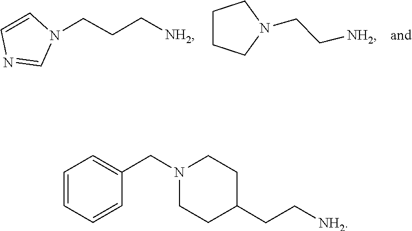

In some embodiments the Head is selected from the group consisting of

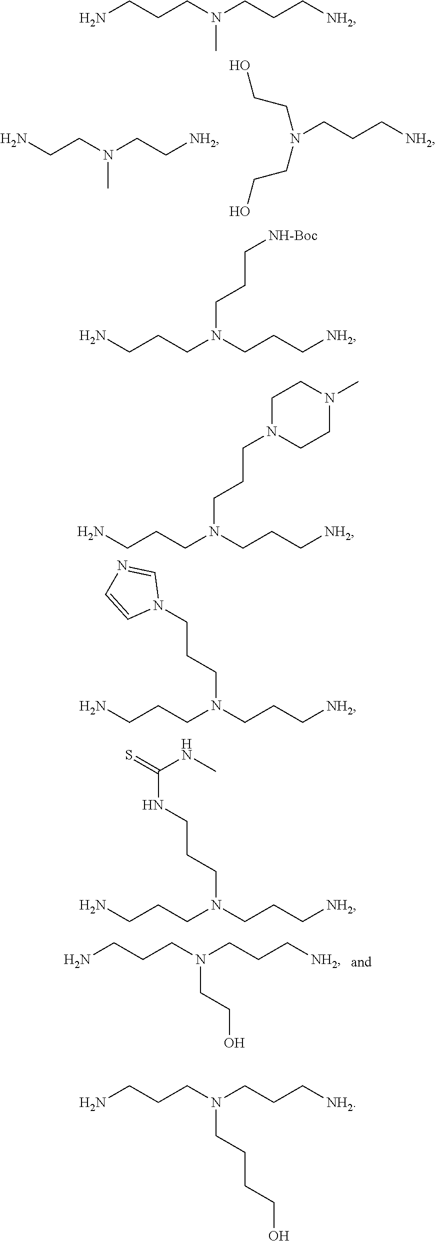

In some embodiments of a compound or salt of Formula (III), p is selected from 2 and 4. In some embodiments, p is 2. In some embodiments, p is 4.

In some embodiments of a compound or salt of Formula (III) or (I), R^{Lipid} is represented by a structure in TABLE 1.

In some embodiments of a compound or salt of Formula (III), the compound is represented by a structure in TABLE 2.

In some aspects, provided herein is a composition comprising a pharmaceutical agent (or therapeutic agent) assembled with a lipid composition. In some embodiments, the lipid composition comprises an ionizable lipid provided herein.

In some aspects, provided herein is a composition, comprising a pharmaceutical agent assembled with a lipid composition comprising an ionizable lipid represented by Formula (III):

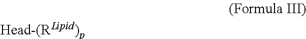

or a pharmaceutically acceptable salt thereof;
wherein:
the Head is selected from

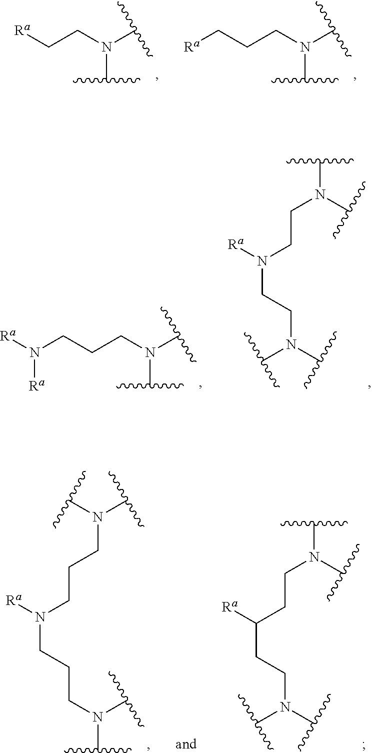

wherein each R^{a} is independently selected from H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₃-C₇ carbocycle, and 3- to 7-membered heterocycle;
each R^{Lipid} is independently selected from:

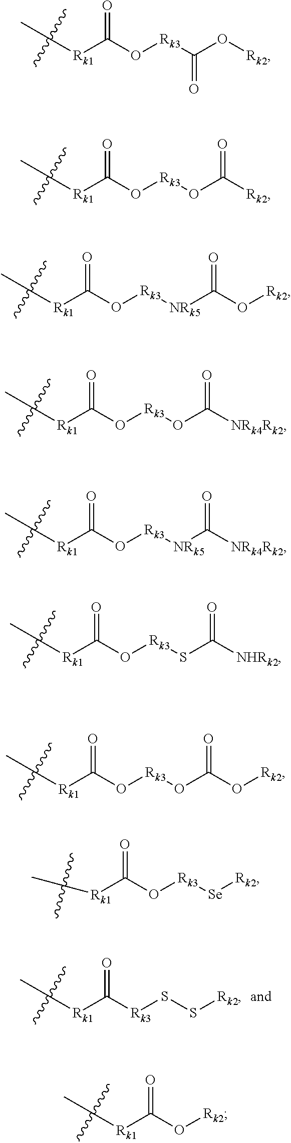

each of Rₖ₁ and Rₖ₃ are independently selected from C₁-C₁₀ alkyl;
each Rₖ₂ is independently selected from C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl;
each Rₖ₄ and Rₖ₅ are independently selected from H and C₁-C₁₀ alkyl; and
p is selected from 1, 2, 3, 4, 5, and 6.

In some embodiments, the pharmaceutical agent or therapeutic agent comprises a peptide, a protein, or a nucleic acid molecule.

In some embodiments, the ionizable lipid comprises an amine head group and at least one hydrophobic tail R^{Lipid} having a structure of Formula (I):

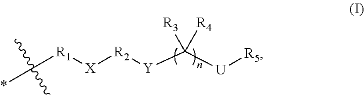

or a pharmaceutically acceptable salt thereof;
   wherein:
      * indicates a point of attachment to a nitrogen in the amine head group;
   R₁ and R₂ are each independently selected from C₁-C₁₂ alkylene, C₁-C₁₂ alkenylene, and 1- to 12-membered heteroalkylene;
   X is

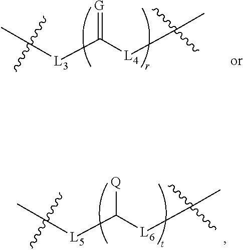

in which each of L₃, L₄, L₅, and L₆ is, independently, a bond, O, S, or NR^{c}; G is 0, S, or NR^{d}; Q is OR^{e}, SR^{f}, or NR^{g}R^{h}; r is selected from 1, 2, 3, 4, 5, and 6; and t is t is selected from 1, 2, 3, 4, 5, and 6; each of R^{e}, R^{d}, R^{e}, R^{f}, R^{g}, and R^{h} is independently H, C1-C10 alkyl, 1- to 10-membered heteroalkyl, aryl, or heteroaryl;
Y and U are each independently a bond, O, S, NR¹⁰, or Se;
n is 0 or 1;
R₃ and R₄, are each independently H, C1-C10 alkyl, 1- to 10-membered heteroalkyl, aryl, or heteroaryl; or R₃ and R₄ together with the atom to which they are attached, form ═O;
R₅ is a C1-C20 alkyl, C2-C20 alkenyl, C2-C20 alkynyl, C3-C20 cycloalkyl, 1- to 20-membered heteroalkyl, 3- to 20-membered heterocycloalkyl, aryl, or heteroaryl; and
the pharmaceutical agent comprises a nucleic acid molecule (e.g., DNA or RNA) encoding at least one therapeutic peptide. In some embodiments, the pharmaceutical agent can be an mRNA encoding a therapeutic peptide suitable for immunotherapy.

In some embodiments, n is 0. In some embodiments, n is 1.

In some embodiments, n is 1 and R₃ and R₄ together with the atom to which they are attached, form ═O. In some embodiments, Y is CH₂ and U is O. In some embodiments, Y and U are both O. In some embodiments, U is CH₂ and Y is O. In some embodiments, Y and U are NR¹⁰. In some embodiments, Y is O and U is NR¹⁰. In some embodiments, Y is S and U is NR¹⁰.

In some embodiments, n is 0, and Y and U are both S. In some embodiments, n is 0, and one of Y and U is O.

In some embodiments, n is 0, and one of Y and U is Se.

In some embodiments, R₁ is a C1-C12 alkyl, linear or branched. In some embodiments, R₁ is a C1-C10 alkyl, linear or branched. In some embodiments, R₁ is a C1-C8 alkyl, linear or branched. In some embodiments, R₁ is a C1-C6 alkyl, linear or branched. In some embodiments, R₁ is a C1-C4 alkyl, linear or branched. In some embodiments, R₁ is a C2 alkyl, e.g.,

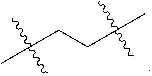 [SMILES: *CC*]

In some embodiments, R₁ is a C3 alkyl, e.g.,

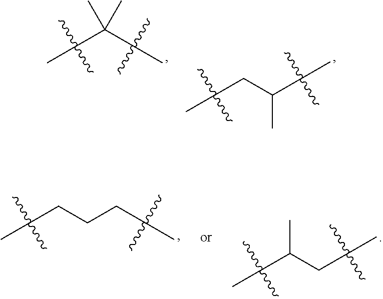

In some embodiments, R₁ is a C4 alkyl. In some embodiments, R₁ is a 1- to 12-membered heteroalkylene.

In some embodiments, R₂ is a C1-C12 alkyl, linear or branched. In some embodiments, R₂ is a C1-C10 alkyl, linear or branched. In some embodiments, R₂ is a C1-C8 alkyl, linear or branched. In some embodiments, R₂ is a C1-C6 alkyl, linear or branched. In some embodiments, R₂ is a C1-C4 alkyl, linear or branched. In some embodiments, R₂ is a C2 alkyl, e.g.,

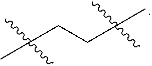 [SMILES: *CC*]

In some embodiments, R₂ is a C3 alkyl, e.g.,

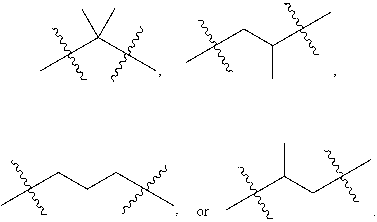

In some embodiments, R₂ is a C4 alkyl. In some embodiments, R₂ is a 1- to 12-membered heteroalkylene.

In some embodiments, the amine head group is represented by

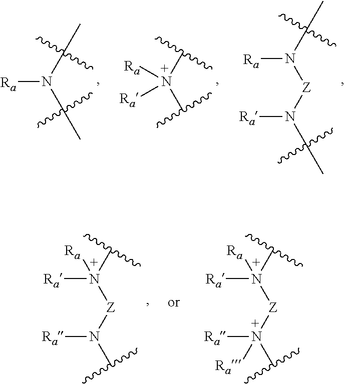

wherein Ra, Ra′, Ra″, and Ra′″ are each independently H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₆-C₂₀ aryl, and 6- to 20-membered heteroaryl, or a R^{Lipid}

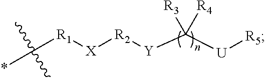

and Z is selected from C₁-C₂₀ alkylene, a 1- to 20-membered heteroalkylene, arylene, and heteroarylene.

In some embodiments, the ionizable lipid is represented by Formula (II):

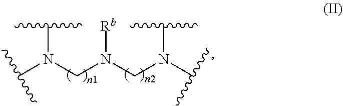

or a pharmaceutically acceptable salt thereof, wherein:
   i) R is a substituted or unsubstituted C1-C6 alkyl, hydroxyalkyl, alkoxyalkyl, or aryl; and
   ii) n1 and n2 are each independently 1, 2, 3, 4, 5, or 6.

In some embodiments, R is a C1-C6 alkyl, linear or branched. In some embodiments, R is a substituted C1-C6 alkyl, linear or branched. In some embodiments, a substituent comprises hydroxyl, carbonyl, thiocarbonyl, alkoxyl, phosphoryl, phosphate, phosphonate, phosphinate, amino, amide, cyclic amine, amidine, imine, cyano, nitro, azido, sulfhydryl, alkylthio, sulfate, sulfonate, sulfamoyl, sulfonamido, sulfonyl, heterocyclyl, aralkyl, or aromatic or heteroaromatic moiety. In some embodiments, the substituent comprises hydroxyl, NH-Boc,

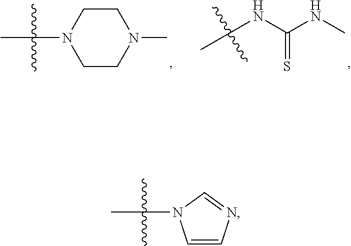

or any suitable substituent thereof.

In some embodiments, n1 is 1, 2, 3, 4, 5, or 6. In some embodiments, n1 is 2 or 3.

In some embodiments, n2 is 1, 2, 3, 4, 5, or 6. In some embodiments, n2 is 2 or 3.

In some embodiments, n1 and n2 are identical. In some embodiments, n1 and n2 are different.

In some embodiments, both n1 and n2 are 2. In some embodiments, both n1 and n2 are 3. In some embodiments, both n1 and n2 are 4.

In some embodiments, the amine head group is selected from the group consisting of

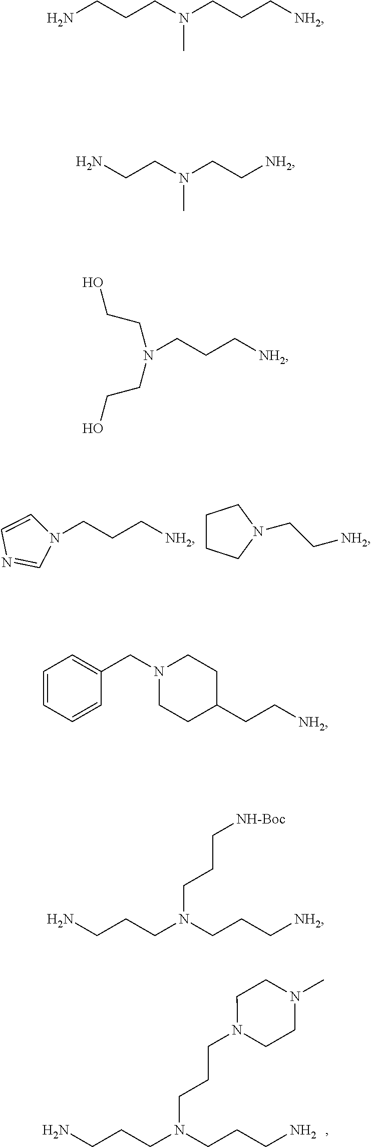

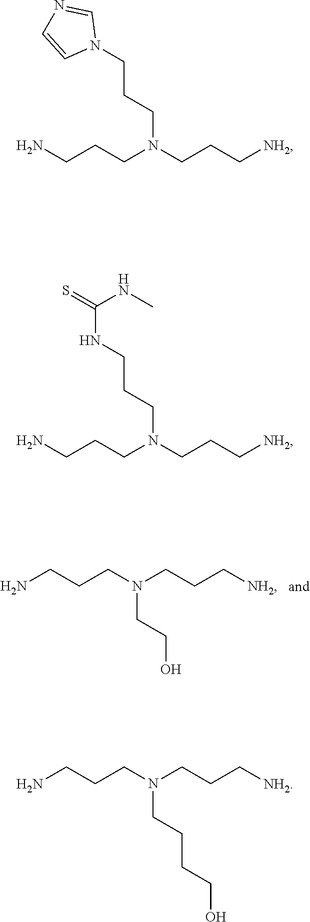 [SMILES: NCCCN(CCCN)CCCn1ccnc1 ; CNC(=S)NCCCN(CCCN)CCCN ; NCCCN(CCO)CCCN ; "and" ; NCCCN(CCCN)CCCCO]

In some embodiments, the at least one hydrophobic tail has a structure of

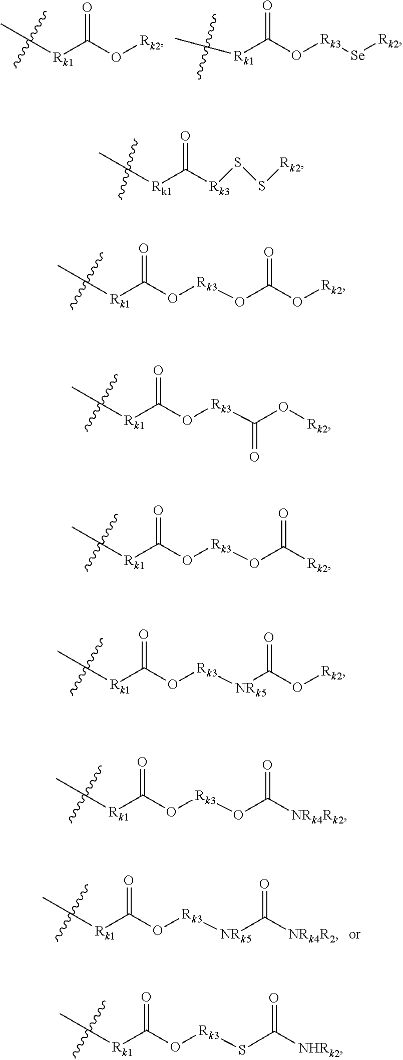

wherein
each Rₖ₁ and Rₖ₃ is independently selected from C₁-C₁₀ alkyl;
each Rₖ₂ is independently selected from C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl; and
each Rₖ₄ and Rₖ₅ is independently selected from H and C₁-C₁₀ alkyl.

In some embodiments, Rₖ₁ is a C1-C10 alkyl, linear or branched. In some embodiments, Rₖ₁ is a C1-C4 alkyl, linear or branched. In some embodiments, Rₖ₁ is a C2 alkyl, e.g.,

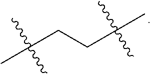 [SMILES: *CC*]

In some embodiments, Rₖ₁ is a C3 alkyl, e.g.,

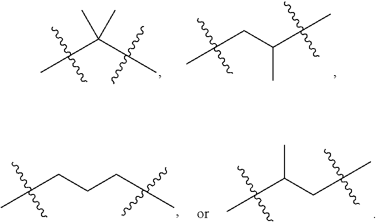

In some embodiments, Rₖ₁ is a C4 alkyl.

In some embodiments, Rₖ₃ is a C1-C10 alkyl, linear or branched. In some embodiments, Rₖ₃ is a C1-C4 alkyl, linear or branched. In some embodiments, Rₖ₃ is a C2 alkyl, e.g.,

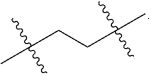 [SMILES: *CC*]

In some embodiments, Rₖ₃ is a C3 alkyl, e.g.,

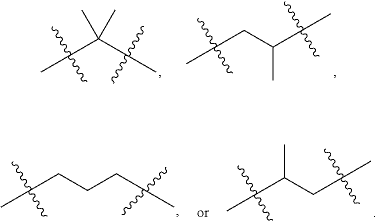

In some embodiments, Rₖ₃ is a C4 alkyl.

In some embodiments, Rₖ₄ is H. In some embodiments, Rₖ₄ is C1-C10 alkyl. In some embodiments, Rₖ₄ is C1-C4 alkyl. In some embodiments, Rₖ₄ is C4-C10 alkyl.

In some embodiments, Rₖ₅ is H. In some embodiments, Rₖ₅ is C1-C10 alkyl. In some embodiments, Rₖ₅ is C1-C4 alkyl. In some embodiments, Rₖ₅ is C4-C10 alkyl.

In some embodiments, Rₖ₂ is a C1-C20 alkyl. In some embodiments, Rₖ₂ is a C2-C20 alkenyl. In some embodiments, Rₖ₂ is a C2-C20 alkynyl. In some embodiments, Rₖ₂ is a C3-C20 cycloalkyl. In some embodiments, Rₖ₂ is a C1-C20 heteroalkyl. In some embodiments, Rₖ₂ is a 1- to 20-membered heterocycloalkyl, aryl, or heteroaryl. In some embodiments, Rₖ₂ is a C4-C20 alkyl, linear or branched. In some embodiments, Rₖ₂ is a C8-C14 branched alkyl. In some embodiments, Rₖ₂ is a C4-C20 alkenyl, linear or branched. In some embodiments, Rₖ₂ is a C8-C14 branched alkenyl.

In some embodiments, the at least one hydrophobic tail is selected from TABLE 1.

TABLE 1
Exemplary Hydrophobic Tail

| Tail # | Tail Structure |
|---|---|
| T0060 | 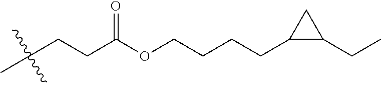 [SMILES: *CCC(=O)OCCCCC1CC1CC] |
| T0061 | 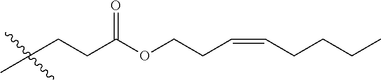 [SMILES: *CCC(=O)OCC/C=C\CCCC] |
| T0062 | 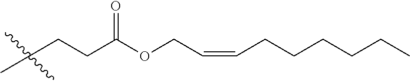 [SMILES: *CCC(=O)OC/C=C\CCCCCC] |
| T0063 | 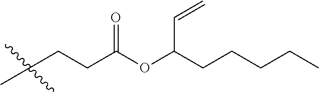 [SMILES: *CCC(=O)OC(C=C)CCCCC] |
| T0064 | 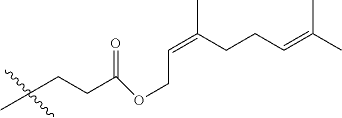 [SMILES: *CCC(=O)OC/C=C(/C)CCC=C(C)C] |
| T0007 | 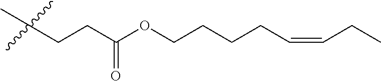 [SMILES: *CCC(=O)OCCCC/C=C\CC] |
| T0114 | 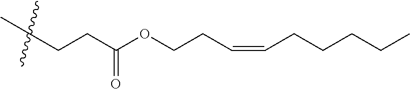 [SMILES: *CCC(=O)OCC/C=C\CCCCC] |
| T0115 | 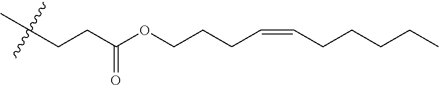 [SMILES: *CCC(=O)OCCC/C=C\CCCCC] |
| T0088 | 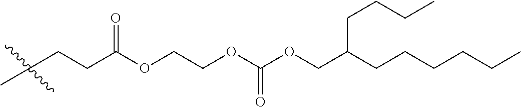 [SMILES: *CCC(=O)OCCOC(=O)OCC(CCCC)CCCCCC] |
| T0075 | 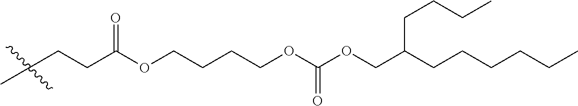 [SMILES: *CCC(=O)OCCCCOC(=O)OCC(CCCC)CCCCCC] |
| T0076 | 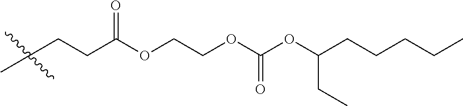 [SMILES: *CCC(=O)OCCOC(=O)OC(CC)CCCCC] |
| T0077 | 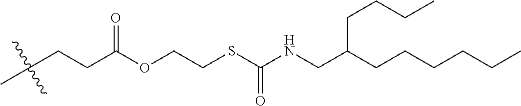 [SMILES: *CCC(=O)OCCSC(=O)NCC(CCCC)CCCCCC] |
| T0122 | 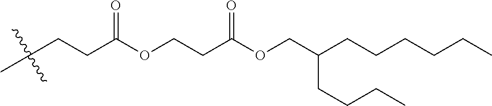 [SMILES: *CCC(=O)OCCC(=O)OCC(CCCC)CCCCCC] |
| T0128 | 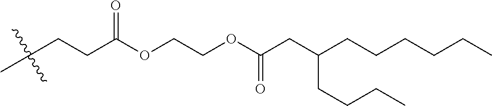 [SMILES: *CCC(=O)OCCOC(=O)CC(CCCC)CCCCCC] |
| T0082 | 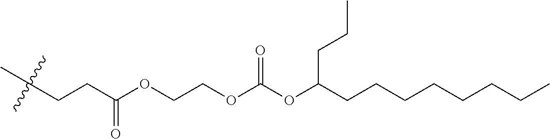 [SMILES: *CCC(=O)OCCOC(=O)OC(CCC)CCCCCCCC] |
| T0083 | 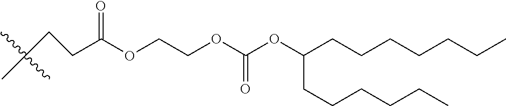 [SMILES: *CCC(=O)OCCOC(=O)OC(CCCCCC)CCCCCCC] |
| T0084 | 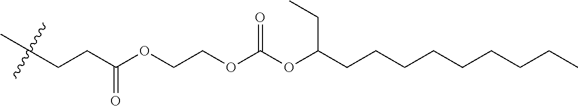 [SMILES: *CCC(=O)OCCOC(=O)OC(CC)CCCCCCCCC] |
| T0094 | 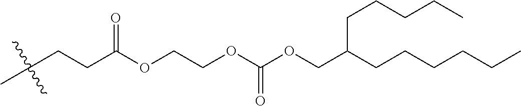 [SMILES: *CCC(=O)OCCOC(=O)OCC(CCCCC)CCCCCC] |
| T0096 | 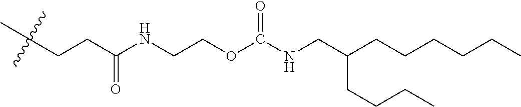 [SMILES: *CCC(=O)NCCOC(=O)NCC(CCCC)CCCCCC] |
| T0065 | 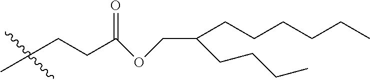 [SMILES: *CCC(=O)OCC(CCCC)CCCCCC] |
| T0066 | 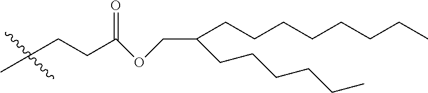 [SMILES: *CCC(=O)OCC(CCCCCC)CCCCCCCC] |
| T0068 | 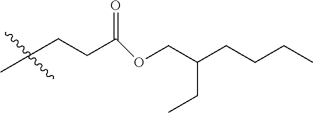 [SMILES: *CCC(=O)OCC(CC)CCCC] |
| T0069 | 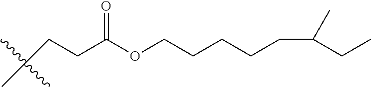 [SMILES: *CCC(=O)OCCCCCC(C)CC] |
| T0070 | 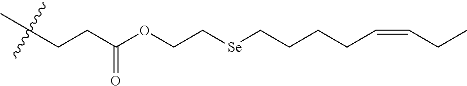 [SMILES: *CCC(=O)OCC[Se]CCCC/C=C\CC] |
| T0071 | 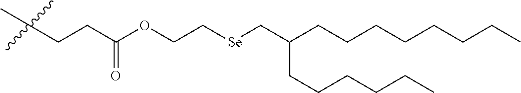 [SMILES: *CCC(=O)OCC[Se]CC(CCCCCC)CCCCCCCC] |
| T0072 | 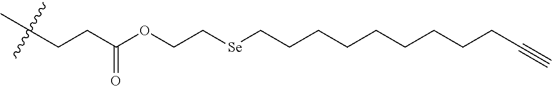 [SMILES: *CCC(=O)OCC[Se]CCCCCCCCCC#C] |
| T0079 | 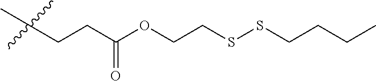 [SMILES: *CCC(=O)OCCSSCCCC] |
| T0089 | 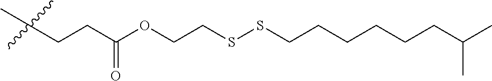 [SMILES: *CCC(=O)OCCSSCCCCCCC(C)C] |
| T0092 | 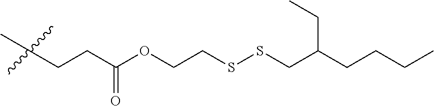 [SMILES: *CCC(=O)OCCSSCC(CC)CCCC] |
| T0093 | 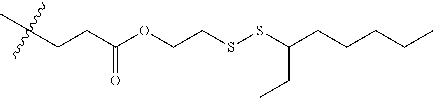 [SMILES: *CCC(=O)OCCSSC(CC)CCCCC] |
| T0106 | 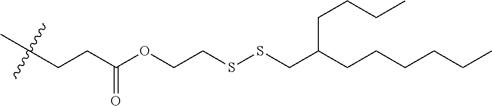 [SMILES: *CCC(=O)OCCSSCC(CCCC)CCCCCC] |
| T0108 | 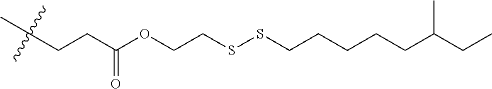 [SMILES: *CCC(=O)OCCSSCCCCCC(C)CC] |
| T0110 | 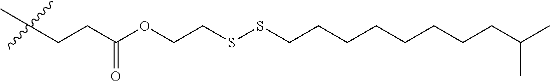 [SMILES: *CCC(=O)OCCSSCCCCCCCCC(C)C] |
| T0113 | 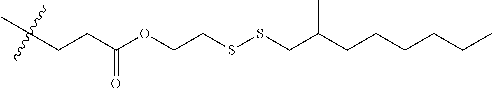 [SMILES: *CCC(=O)OCCSSCC(C)CCCCCC] |
| T0001 | 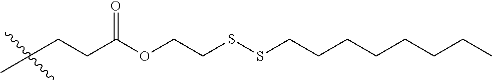 [SMILES: *CCC(=O)OCCSSCCCCCCCC] |
| T0097 | 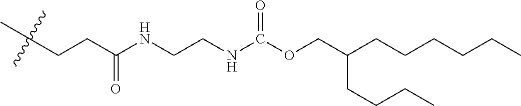 [SMILES: *CCC(=O)NCCNC(=O)OCC(CCCC)CCCCCC] |
| T0100 | 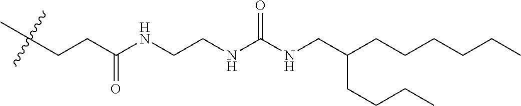 [SMILES: *CCC(=O)NCCNC(=O)NCC(CCCC)CCCCCC] |

In some embodiments, the ionizable lipid comprises at least two hydrophobic tails. In some embodiments, the at least two hydrophobic tails are independently of structure

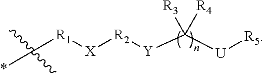

In some embodiments, the at least two hydrophobic tails are identical. In some embodiments, the at least two hydrophobic tails are not identical. In some embodiments, one of the at least two hydrophobic tails is different from the rest.

In some embodiments, the lipid composition comprises at least three hydrophobic tails. In some embodiments, the at least three hydrophobic tails are independently of structure

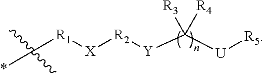

In some embodiments, the at least three hydrophobic tails are identical. In some embodiments, the at least three hydrophobic tails are not identical. In some embodiments, one of the at least three hydrophobic tails is different from the rest.

In some embodiments, the lipid composition comprises two hydrophobic tails. In some embodiments, the two hydrophobic tails are independently of structure

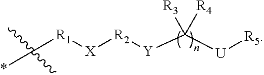

In some embodiments, the two hydrophobic tails are identical. In some embodiments, the two hydrophobic tails are not identical.

In some embodiments, the lipid composition comprises three hydrophobic tails. In some embodiments, the three hydrophobic tails are independently of structure

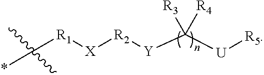

In some embodiments, the three hydrophobic tails are identical. In some embodiments, two of the three hydrophobic tails are identical and the third hydrophobic tail is different. In some embodiments, all three hydrophobic tails are different.

In some embodiments, the lipid composition comprises four hydrophobic tails. In some embodiments, the four hydrophobic tails are independently of structure

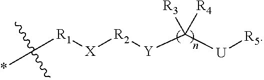

In some embodiments, the four hydrophobic tails are identical. In some embodiments, three of the four hydrophobic tails are identical and the fourth hydrophobic tail is different. In some embodiments, two of the four hydrophobic tails are identical, the other two hydrophobic tails are identical, and the two are different from other two. In some embodiments, two of the four hydrophobic tails are identical while the other two are different from each other and are different from the two. In some embodiments, all four hydrophobic tails are different.

In some embodiments, the ionizable lipid is selected from TABLE 2.

TABLE 2
Exemplary Ionizable Lipid

| Lipid# | Lipid Structure |
|---|---|
| L0522 | 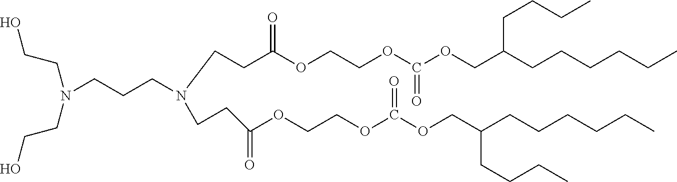 [SMILES: CCCCCCC(CCCC)COC(=O)OCCOC(=O)CCN(CCCN(CCO)CCO)CCC(=O)OCCOC(=O)OCC(CCCC)CCCCCC] |
| L0524 | 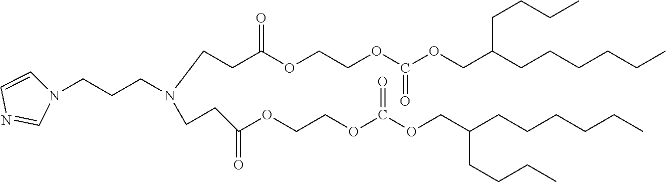 [SMILES: CCCCCCC(CCCC)COC(=O)OCCOC(=O)CCN(CCCn1ccnc1)CCC(=O)OCCOC(=O)OCC(CCCC)CCCCCC] |
| L0526 | 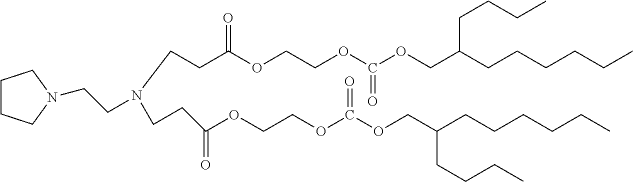 [SMILES: CCCCCCC(CCCC)COC(=O)OCCOC(=O)CCN(CCC(=O)OCCOC(=O)OCC(CCCC)CCCCCC)CCN1CCCC1] |
| L0539 | 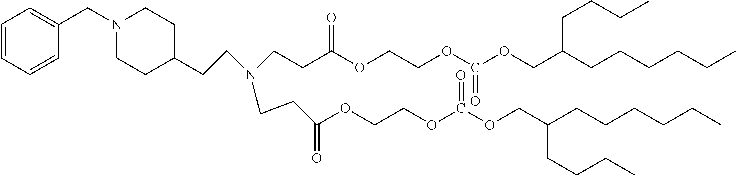 [SMILES: CCCCCCC(CCCC)COC(=O)OCCOC(=O)CCN(CCC(=O)OCCOC(=O)OCC(CCCC)CCCCCC)CCC1CCN(Cc2ccccc2)CC1] |
| L0668 | 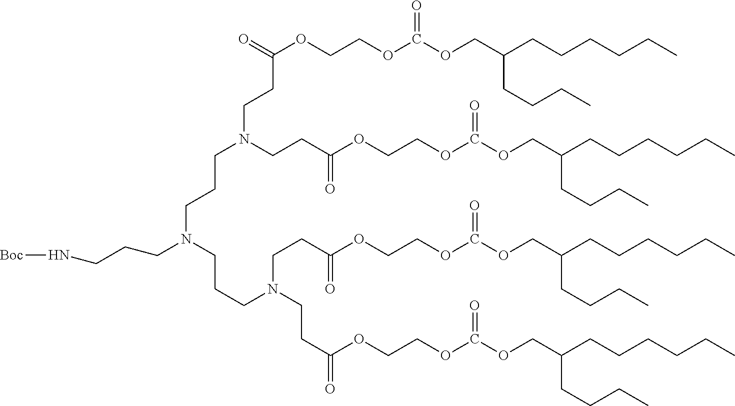 [SMILES: CCCCCCC(CCCC)COC(=O)OCCOC(=O)CCN(CCCN(CCCNC(=O)OC(C)(C)C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0674 | 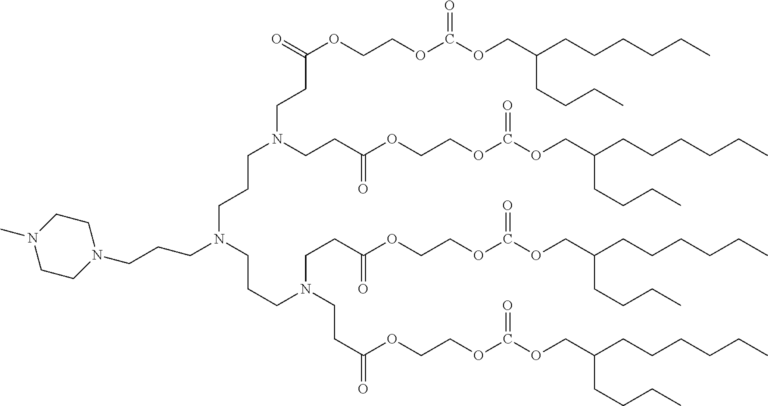 [SMILES: CCCCCCC(CCCC)COC(=O)OCCOC(=O)CCN(CCCN(CCCN(CCC(=O)OCCOC(=O)OCC(CCCC)CCCCCC)CCC(=O)OCCOC(=O)OCC(CCCC)CCCCCC)CCCN1CCN(C)CC1)CCC(=O)OCCOC(=O)OCC(CCCC)CCCCCC] |
| L0773 | 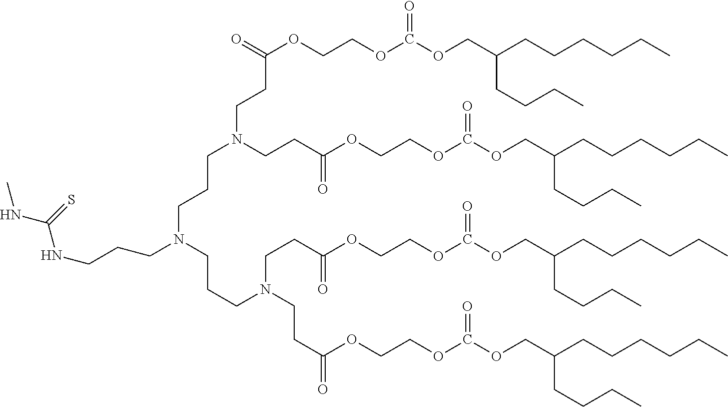 [SMILES: CCCCCCC(CCCC)COC(=O)OCCOC(=O)CCN(CCCN(CCCNC(=S)NC)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0665 | 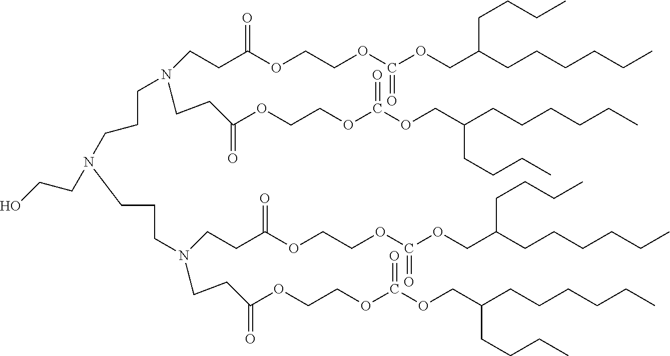 [SMILES: CCCCCCC(CCCC)COC(=O)OCCOC(=O)CCN(CCCN(CCO)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0666 | 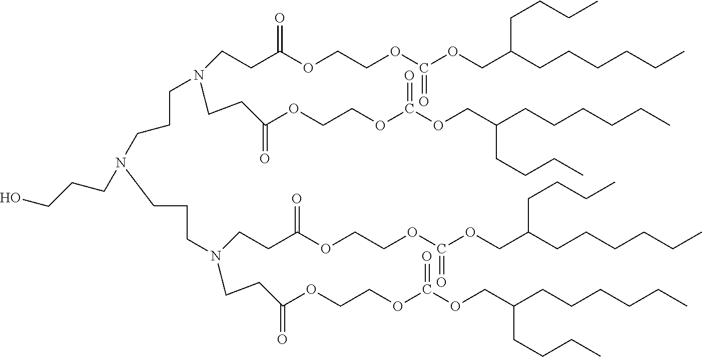 [SMILES: CCCCCCC(CCCC)COC(=O)OCCOC(=O)CCN(CCCN(CCCO)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0667 | 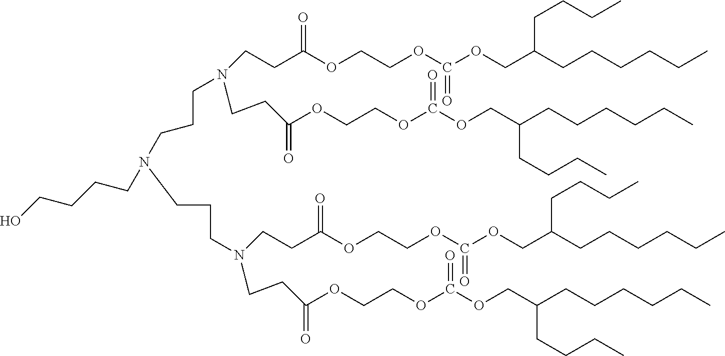 [SMILES: CCCCCCC(CCCC)COC(=O)OCCOC(=O)CCN(CCCN(CCCCO)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0586 | 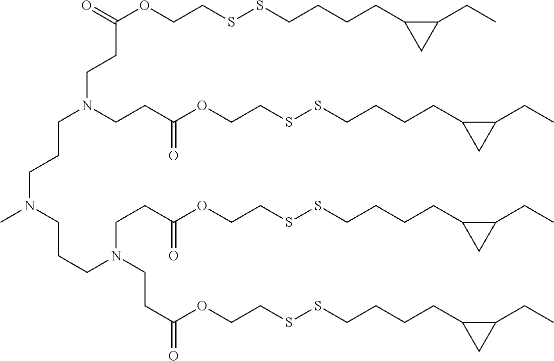 [SMILES: CCC1CC1CCCCSSCCOC(=O)CCN(CCCN(C)CCCN(CCC(=O)OCCSSCCCCC1CC1CC)CCC(=O)OCCSSCCCCC1CC1CC)CCC(=O)OCCSSCCCCC1CC1CC] |
| L0587 | 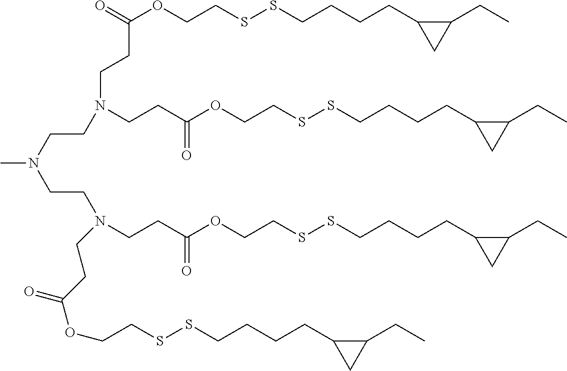 [SMILES: CCC1CC1CCCCSSCCOC(=O)CCN(CCC(=O)OCCSSCCCCC1CC1CC)CCN(C)CCN(CCC(=O)OCCSSCCCCC1CC1CC)CCC(=O)OCCSSCCCCC1CC1CC] |
| L0588 | 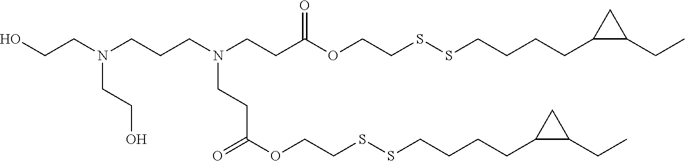 [SMILES: CCC1CC1CCCCSSCCOC(=O)CCN(CCCN(CCO)CCO)CCC(=O)OCCSSCCCCC1CC1CC] |
| L0589 | 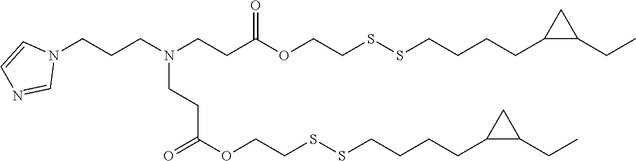 [SMILES: CCC1CC1CCCCSSCCOC(=O)CCN(CCCn1ccnc1)CCC(=O)OCCSSCCCCC1CC1CC] |
| L0591 | 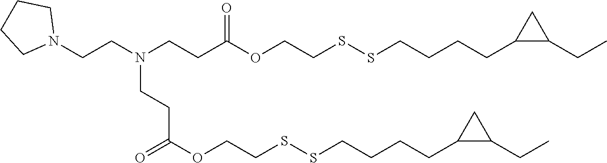 [SMILES: CCC1CC1CCCCSSCCOC(=O)CCN(CCC(=O)OCCSSCCCCC1CC1CC)CCN1CCCC1] |
| L0826 | 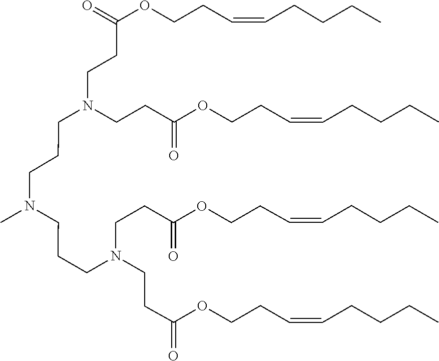 [SMILES: CCCC/C=C\CCOC(=O)CCN(CCCN(C)CCCN(CCC(=O)OCC/C=C\CCCC)CCC(=O)OCC/C=C\CCCC)CCC(=O)OCC/C=C\CCCC] |
| L0828 | 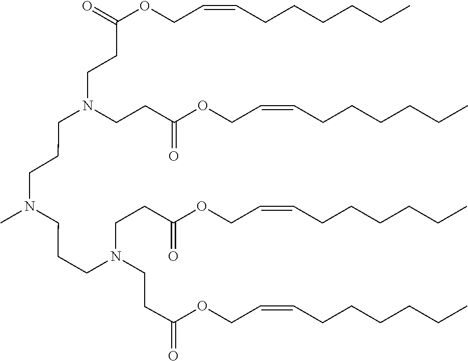 [SMILES: CCCCCC/C=C\COC(=O)CCN(CCCN(C)CCCN(CCC(=O)OC/C=C\CCCCCC)CCC(=O)OC/C=C\CCCCCC)CCC(=O)OC/C=C\CCCCCC] |
| L0531 | 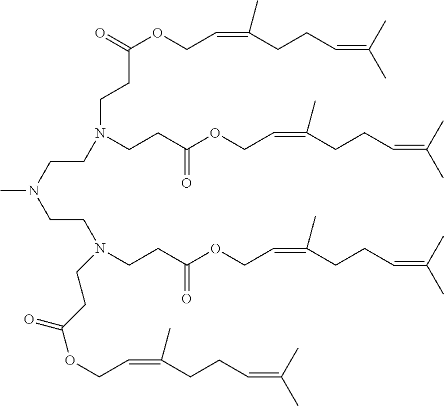 [SMILES: CC(C)=CCC/C(C)=C\COC(=O)CCN(CCC(=O)OC/C=C(/C)CCC=C(C)C)CCN(C)CCN(CCC(=O)OC/C=C(/C)CCC=C(C)C)CCC(=O)OC/C=C(/C)CCC=C(C)C] |
| L0534 | 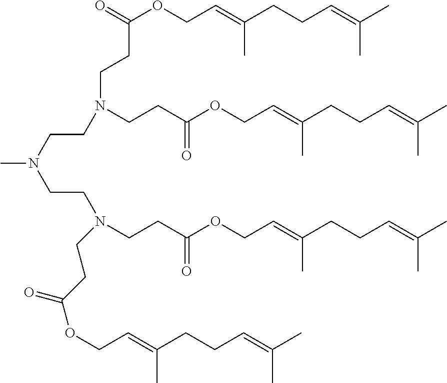 [SMILES: CC(C)=CCC/C(C)=C/COC(=O)CCN(CCC(=O)OC/C=C(\C)CCC=C(C)C)CCN(C)CCN(CCC(=O)OC/C=C(\C)CCC=C(C)C)CCC(=O)OC/C=C(\C)CCC=C(C)C] |
| L0823 | 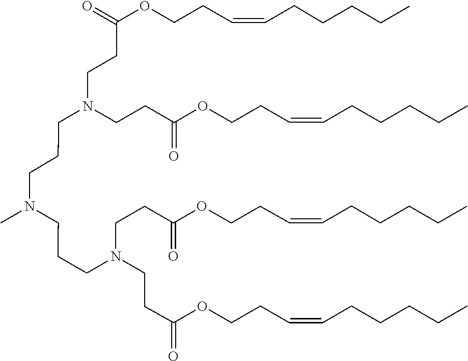 [SMILES: CCCCC/C=C\CCOC(=O)CCN(CCCN(C)CCCN(CCC(=O)OCC/C=C\CCCCC)CCC(=O)OCC/C=C\CCCCC)CCC(=O)OCC/C=C\CCCCC] |
| L0824 | 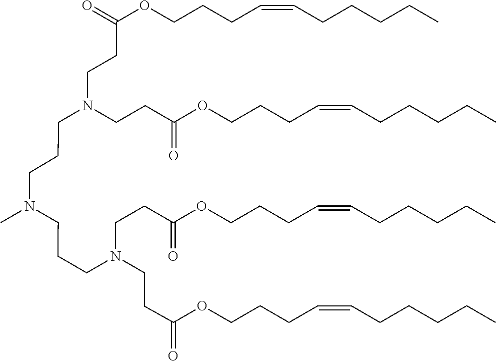 [SMILES: CCCCC/C=C\CCCOC(=O)CCN(CCCN(C)CCCN(CCC(=O)OCCC/C=C\CCCCC)CCC(=O)OCCC/C=C\CCCCC)CCC(=O)OCCC/C=C\CCCCC] |
| L0823 | 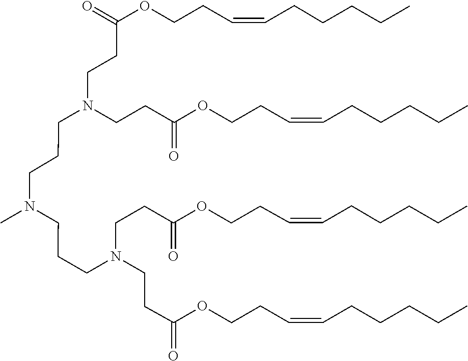 [SMILES: CCCCC/C=C\CCOC(=O)CCN(CCCN(C)CCCN(CCC(=O)OCC/C=C\CCCCC)CCC(=O)OCC/C=C\CCCCC)CCC(=O)OCC/C=C\CCCCC] |
| L0532 | 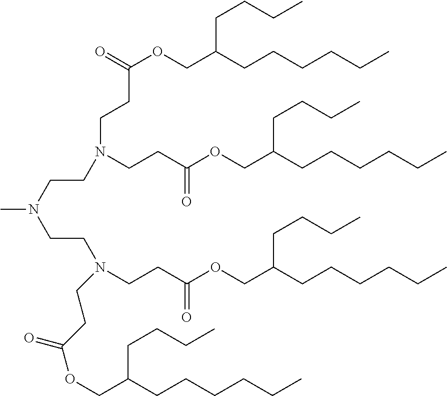 [SMILES: CCCCCCC(CCCC)COC(=O)CCN(CCC(=O)OCC(CCCC)CCCCCC)CCN(C)CCN(CCC(=O)OCC(CCCC)CCCCCC)CCC(=O)OCC(CCCC)CCCCCC] |
| L0533 | 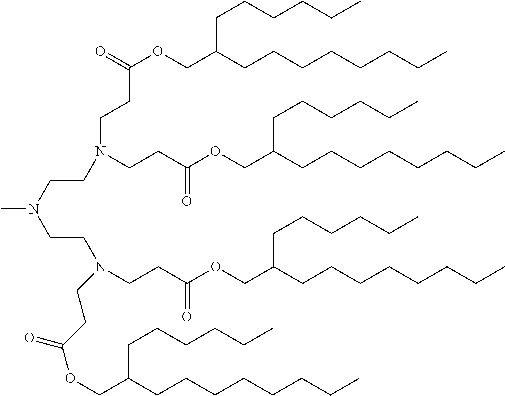 [SMILES: CCCCCCCCC(CCCCCC)COC(=O)CCN(CCC(=O)OCC(CCCCCC)CCCCCCCC)CCN(C)CCN(CCC(=O)OCC(CCCCCC)CCCCCCCC)CCC(=O)OCC(CCCCCC)CCCCCCCC] |
| L0535 | 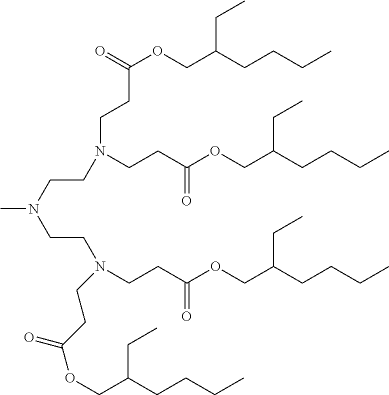 [SMILES: CCCCC(CC)COC(=O)CCN(CCC(=O)OCC(CC)CCCC)CCN(C)CCN(CCC(=O)OCC(CC)CCCC)CCC(=O)OCC(CC)CCCC] |
| L0630 | 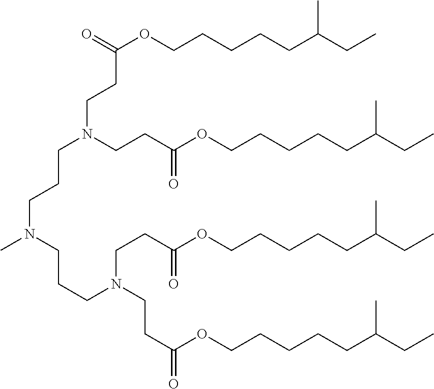 [SMILES: CCC(C)CCCCCOC(=O)CCN(CCCN(C)CCCN(CCC(=O)OCCCCCC(C)CC)CCC(=O)OCCCCCC(C)CC)CCC(=O)OCCCCCC(C)CC] |
| L0631 | 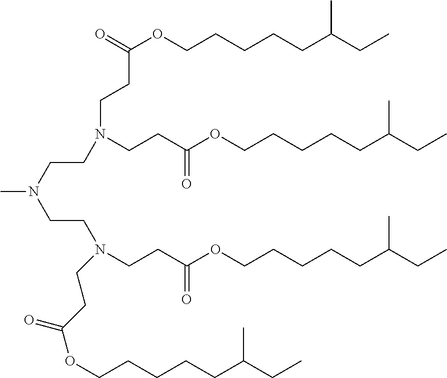 [SMILES: CCC(C)CCCCCOC(=O)CCN(CCC(=O)OCCCCCC(C)CC)CCN(C)CCN(CCC(=O)OCCCCCC(C)CC)CCC(=O)OCCCCCC(C)CC] |
| L0632 | 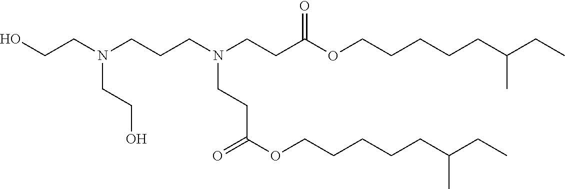 [SMILES: CCC(C)CCCCCOC(=O)CCN(CCCN(CCO)CCO)CCC(=O)OCCCCCC(C)CC] |
| L0633 | 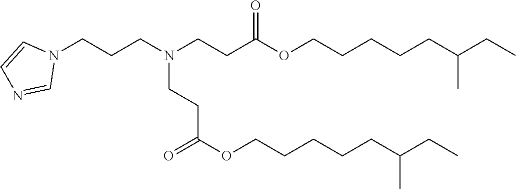 [SMILES: CCC(C)CCCCCOC(=O)CCN(CCCn1ccnc1)CCC(=O)OCCCCCC(C)CC] |
| L0506 | 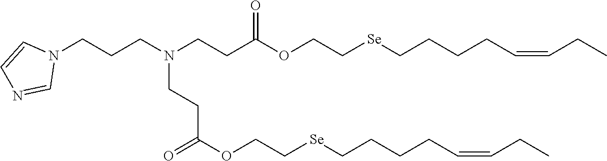 [SMILES: CC/C=C\CCCC[Se]CCOC(=O)CCN(CCCn1ccnc1)CCC(=O)OCC[Se]CCCC/C=C\CC] |
| L0507 | 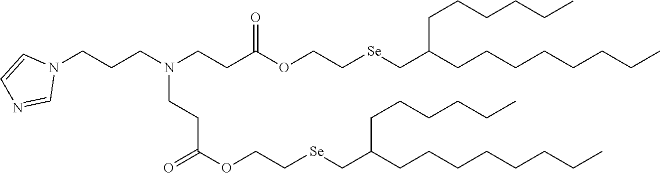 [SMILES: CCCCCCCCC(CCCCCC)C[Se]CCOC(=O)CCN(CCCn1ccnc1)CCC(=O)OCC[Se]CC(CCCCCC)CCCCCCCC] |
| L0508 | 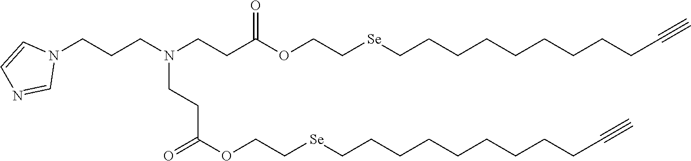 [SMILES: C#CCCCCCCCCC[Se]CCOC(=O)CCN(CCCn1ccnc1)CCC(=O)OCC[Se]CCCCCCCCCC#C] |
| L0509 | 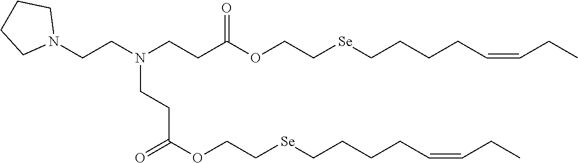 [SMILES: CC/C=C\CCCC[Se]CCOC(=O)CCN(CCC(=O)OCC[Se]CCCC/C=C\CC)CCN1CCCC1] |
| L0510 | 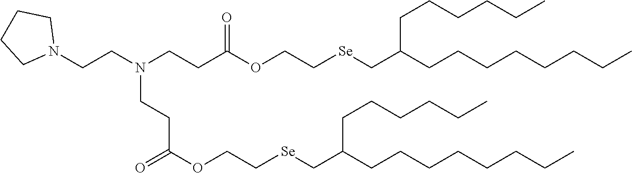 [SMILES: CCCCCCCCC(CCCCCC)C[Se]CCOC(=O)CCN(CCC(=O)OCC[Se]CC(CCCCCC)CCCCCCCC)CCN1CCCC1] |
| L0511 | 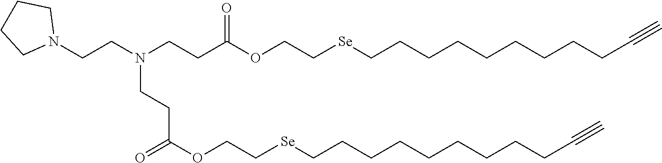 [SMILES: C#CCCCCCCCCC[Se]CCOC(=O)CCN(CCC(=O)OCC[Se]CCCCCCCCCC#C)CCN1CCCC1] |
| L0512 | 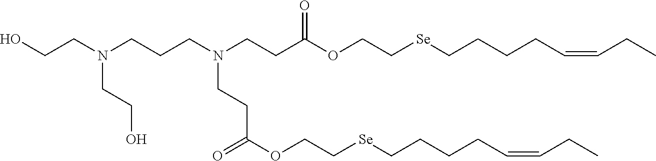 [SMILES: CC/C=C\CCCC[Se]CCOC(=O)CCN(CCCN(CCO)CCO)CCC(=O)OCC[Se]CCCC/C=C\CC] |
| L0513 | 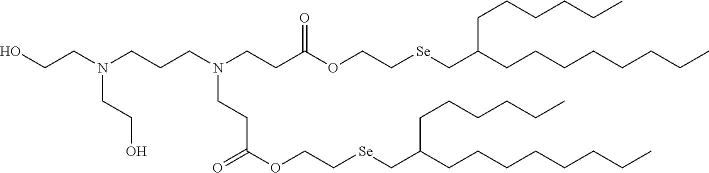 [SMILES: CCCCCCCCC(CCCCCC)C[Se]CCOC(=O)CCN(CCCN(CCO)CCO)CCC(=O)OCC[Se]CC(CCCCCC)CCCCCCCC] |
| L0514 | 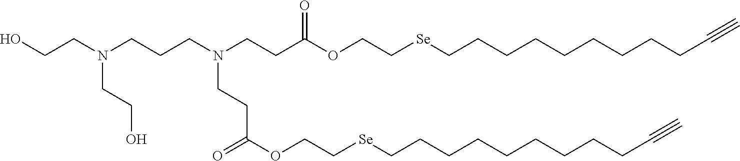 [SMILES: C#CCCCCCCCCC[Se]CCOC(=O)CCN(CCCN(CCO)CCO)CCC(=O)OCC[Se]CCCCCCCCCC#C] |
| L0825 | 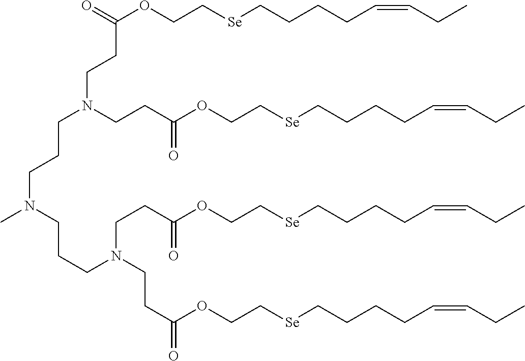 [SMILES: CC/C=C\CCCC[Se]CCOC(=O)CCN(CCCN(C)CCCN(CCC(=O)OCC[Se]CCCC/C=C\CC)CCC(=O)OCC[Se]CCCC/C=C\CC)CCC(=O)OCC[Se]CCCC/C=C\CC] |
| L0580 | 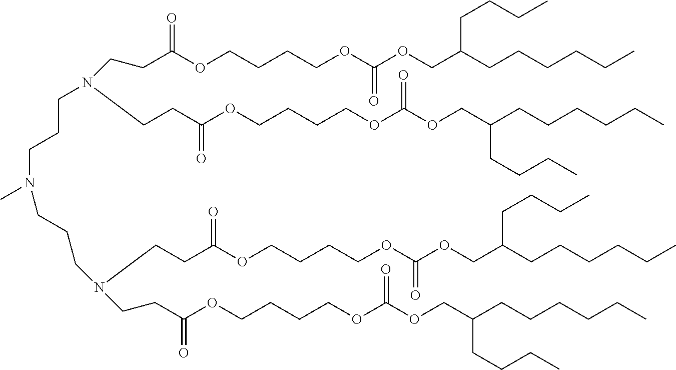 [SMILES: CCCCCCC(CCCC)COC(=O)OCCCCOC(=O)CCN(CCCN(C)CCCN(CCC(=O)OCCCCOC(=O)OCC(CCCC)CCCCCC)CCC(=O)OCCCCOC(=O)OCC(CCCC)CCCCCC)CCC(=O)OCCCCOC(=O)OCC(CCCC)CCCCCC] |
| L0582 | 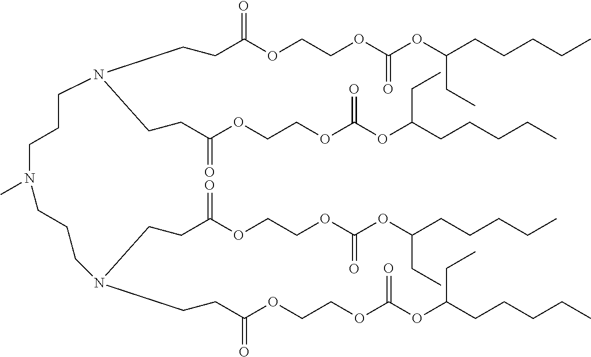 [SMILES: CCCCCC(CC)OC(=O)OCCOC(=O)CCN(CCCN(C)CCCN(CCC(=O)OCCOC(=O)OC(CC)CCCCC)CCC(=O)OCCOC(=O)OC(CC)CCCCC)CCC(=O)OCCOC(=O)OC(CC)CCCCC] |
| L0550 | 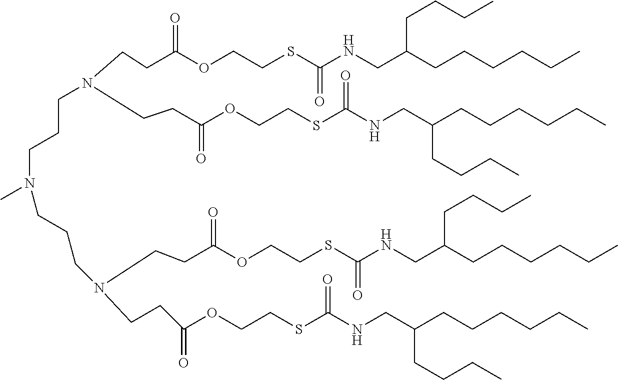 [SMILES: CCCCCCC(CCCC)CNC(=O)SCCOC(=O)CCN(CCCN(C)CCCN(CCC(=O)OCCSC(=O)NCC(CCCC)CCCCCC)CCC(=O)OCCSC(=O)NCC(CCCC)CCCCCC)CCC(=O)OCCSC(=O)NCC(CCCC)CCCCCC] |
| L0551 | 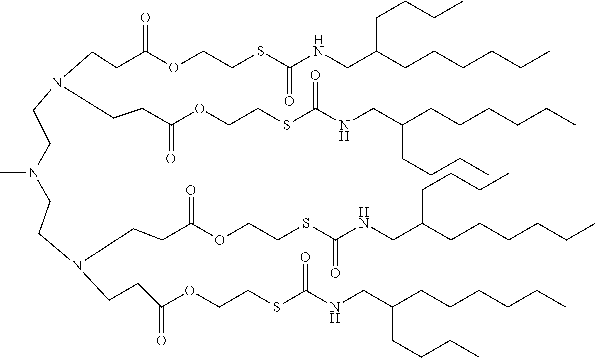 [SMILES: CCCCCCC(CCCC)CNC(=O)SCCOC(=O)CCN(CCC(=O)OCCSC(=O)NCC(CCCC)CCCCCC)CCN(C)CCN(CCC(=O)OCCSC(=O)NCC(CCCC)CCCCCC)CCC(=O)OCCSC(=O)NCC(CCCC)CCCCCC] |
| L0552 | 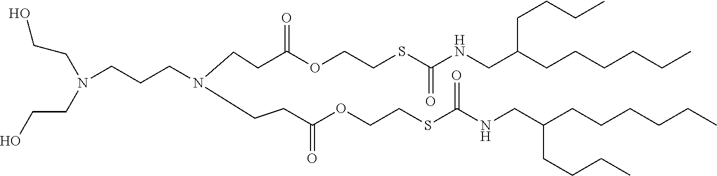 [SMILES: CCCCCCC(CCCC)CNC(=O)SCCOC(=O)CCN(CCCN(CCO)CCO)CCC(=O)OCCSC(=O)NCC(CCCC)CCCCCC] |
| L0553 | 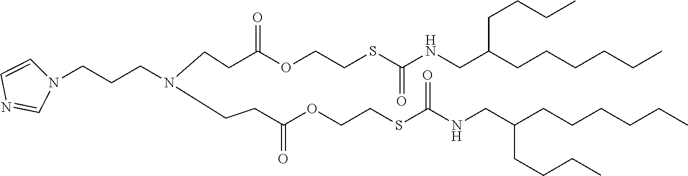 [SMILES: CCCCCCC(CCCC)CNC(=O)SCCOC(=O)CCN(CCCn1ccnc1)CCC(=O)OCCSC(=O)NCC(CCCC)CCCCCC] |
| L0555 | 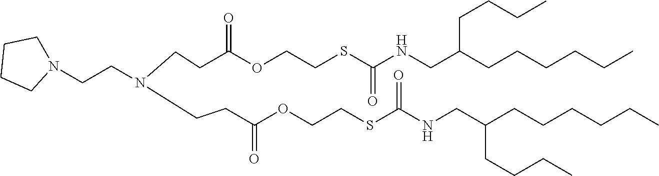 [SMILES: CCCCCCC(CCCC)CNC(=O)SCCOC(=O)CCN(CCC(=O)OCCSC(=O)NCC(CCCC)CCCCCC)CCN1CCCC1] |
| L0637 | 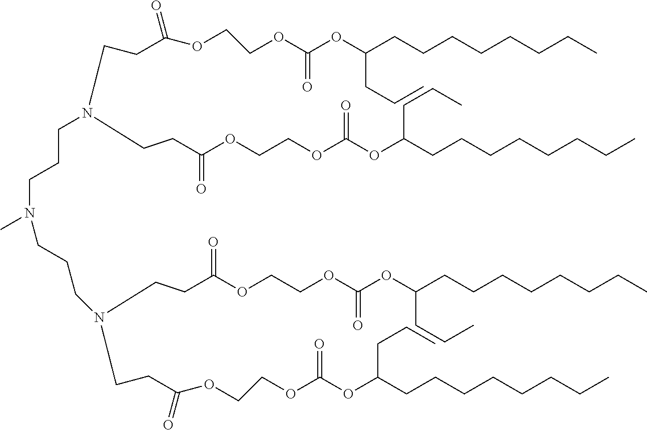 [SMILES: CCCCCCCCC(CCC)OC(=O)OCCOC(=O)CCN(CCCN(C)CCCN(CCC(=O)OCCOC(=O)OC(CCC)CCCCCCCC)CCC(=O)OCCOC(=O)OC(CCC)CCCCCCCC)CCC(=O)OCCOC(=O)OC(CCC)CCCCCCCC] |
| L0638 | 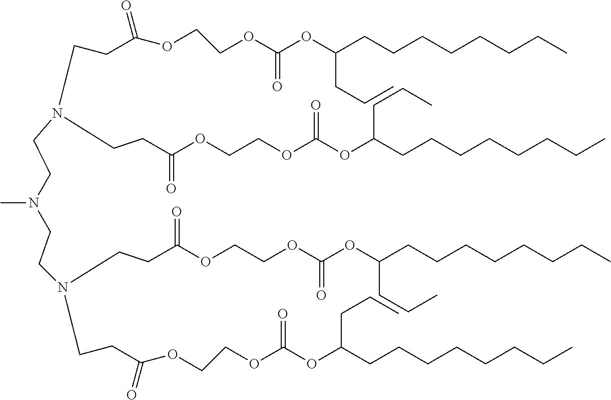 [SMILES: CCCCCCCCC(CCC)OC(=O)OCCOC(=O)CCN(CCC(=O)OCCOC(=O)OC(CCC)CCCCCCCC)CCN(C)CCN(CCC(=O)OCCOC(=O)OC(CCC)CCCCCCCC)CCC(=O)OCCOC(=O)OC(CCC)CCCCCCCC] |
| L0639 | 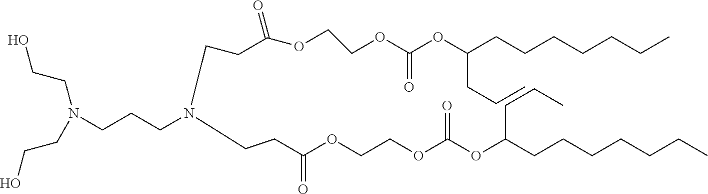 [SMILES: CCCCCCCC(CCC)OC(=O)OCCOC(=O)CCN(CCCN(CCO)CCO)CCC(=O)OCCOC(=O)OC(CCC)CCCCCCC] |
| L0640 | 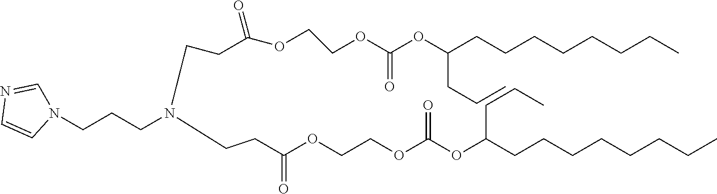 [SMILES: CCCCCCCCC(CCC)OC(=O)OCCOC(=O)CCN(CCCn1ccnc1)CCC(=O)OCCOC(=O)OC(CCC)CCCCCCCC] |
| L0654 | 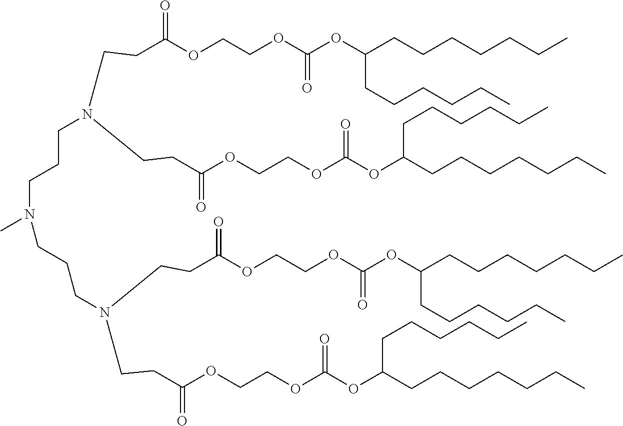 [SMILES: CCCCCCCC(CCCCCC)OC(=O)OCCOC(=O)CCN(CCCN(C)CCCN(CCC(=O)OCCOC(=O)OC(CCCCCC)CCCCCCC)CCC(=O)OCCOC(=O)OC(CCCCCC)CCCCCCC)CCC(=O)OCCOC(=O)OC(CCCCCC)CCCCCCC] |
| L0655 | 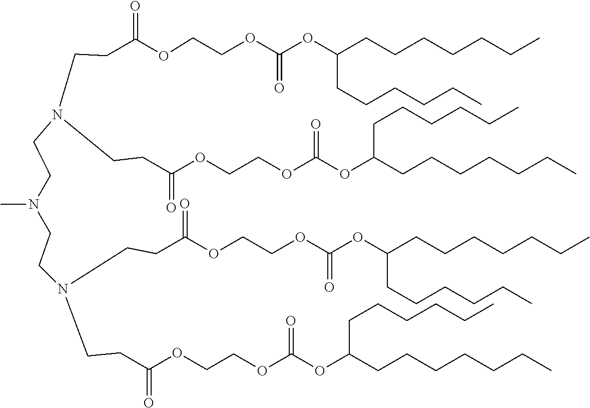 [SMILES: CCCCCCCC(CCCCCC)OC(=O)OCCOC(=O)CCN(CCC(=O)OCCOC(=O)OC(CCCCCC)CCCCCCC)CCN(C)CCN(CCC(=O)OCCOC(=O)OC(CCCCCC)CCCCCCC)CCC(=O)OCCOC(=O)OC(CCCCCC)CCCCCCC] |
| L0656 | 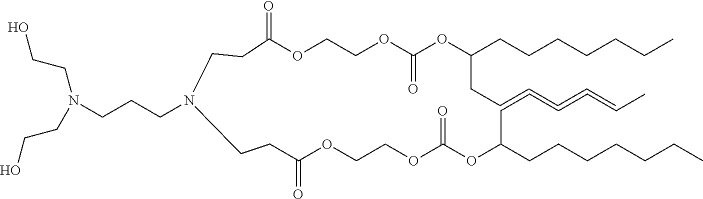 [SMILES: CCCCCCCC(CCCCCC)OC(=O)OCCOC(=O)CCN(CCCN(CCO)CCO)CCC(=O)OCCOC(=O)OC(CCCCCC)CCCCCCC] |
| L0657 | 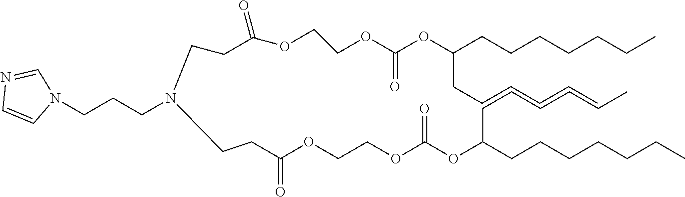 [SMILES: CCCCCCCC(CCCCCC)OC(=O)OCCOC(=O)CCN(CCCn1ccnc1)CCC(=O)OCCOC(=O)OC(CCCCCC)CCCCCCC] |
| L0659 | 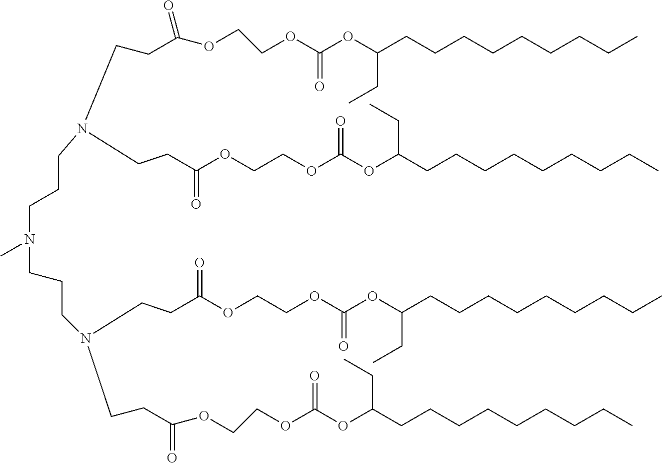 [SMILES: CCCCCCCCCC(CC)OC(=O)OCCOC(=O)CCN(CCCN(C)CCCN(CCC(=O)OCCOC(=O)OC(CC)CCCCCCCCC)CCC(=O)OCCOC(=O)OC(CC)CCCCCCCCC)CCC(=O)OCCOC(=O)OC(CC)CCCCCCCCC] |
| L0660 | 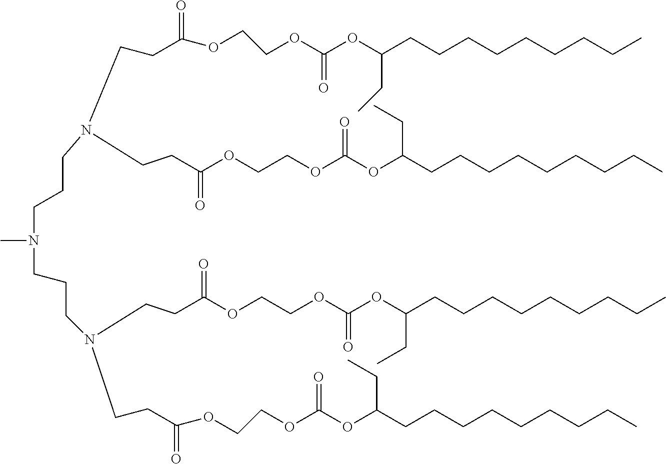 [SMILES: CCCCCCCCCC(CC)OC(=O)OCCOC(=O)CCN(CCCN(C)CCCN(CCC(=O)OCCOC(=O)OC(CC)CCCCCCCCC)CCC(=O)OCCOC(=O)OC(CC)CCCCCCCCC)CCC(=O)OCCOC(=O)OC(CC)CCCCCCCCC] |
| L0661 | 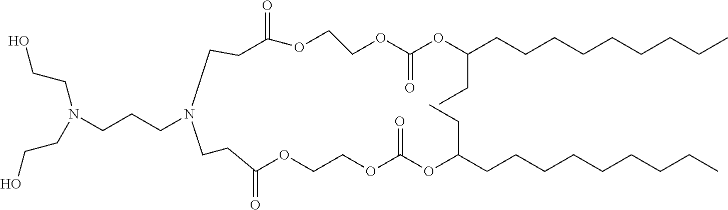 [SMILES: CCCCCCCCCC(CC)OC(=O)OCCOC(=O)CCN(CCCN(CCO)CCO)CCC(=O)OCCOC(=O)OC(CC)CCCCCCCCC] |
| L0662 | 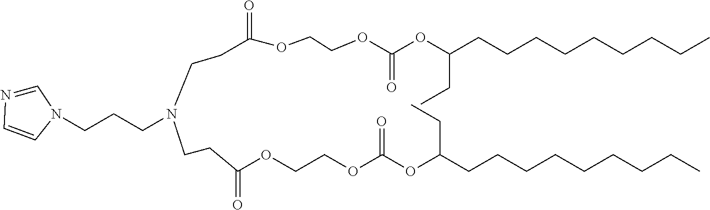 [SMILES: CCCCCCCCCC(CC)OC(=O)OCCOC(=O)CCN(CCCn1ccnc1)CCC(=O)OCCOC(=O)OC(CC)CCCCCCCCC] |
| L0664 | 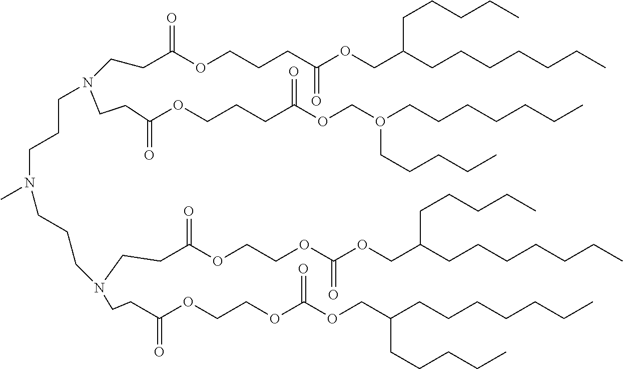 |
| L0739 | 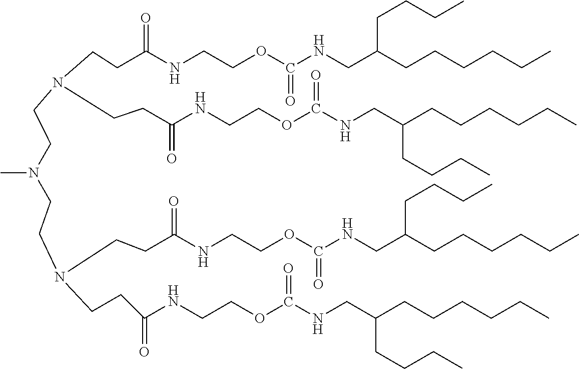 [SMILES: CCCCCCC(CCCC)CNC(=O)OCCNC(=O)CCN(CCC(=O)NCCOC(=O)NCC(CCCC)CCCCCC)CCN(C)CCN(CCC(=O)NCCOC(=O)NCC(CCCC)CCCCCC)CCC(=O)NCCOC(=O)NCC(CCCC)CCCCCC] |
| L0740 | 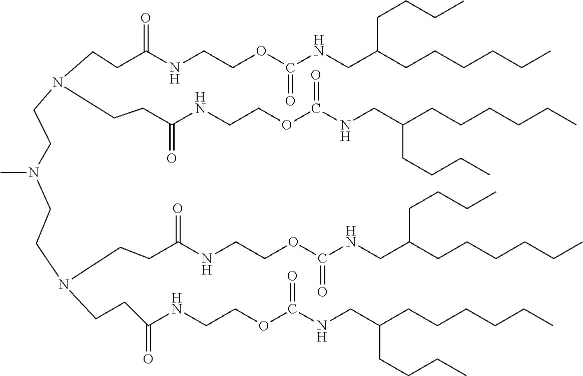 [SMILES: CCCCCCC(CCCC)CNC(=O)OCCNC(=O)CCN(CCC(=O)NCCOC(=O)NCC(CCCC)CCCCCC)CCN(C)CCN(CCC(=O)NCCOC(=O)NCC(CCCC)CCCCCC)CCC(=O)NCCOC(=O)NCC(CCCC)CCCCCC] |
| L0744 | 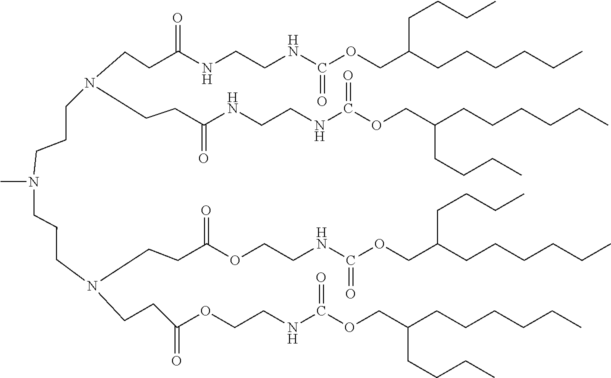 [SMILES: CCCCCCC(CCCC)COC(=O)NCCNC(=O)CCN(CCCN(C)CCCN(CCC(=O)OCCNC(=O)OCC(CCCC)CCCCCC)CCC(=O)OCCNC(=O)OCC(CCCC)CCCCCC)CCC(=O)NCCNC(=O)OCC(CCCC)CCCCCC] |
| L0745 | 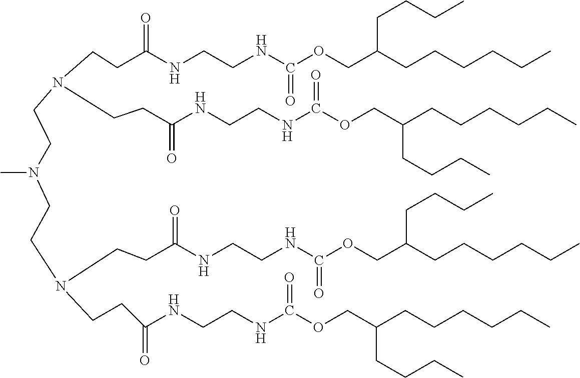 [SMILES: CCCCCCC(CCCC)COC(=O)NCCNC(=O)CCN(CCC(=O)NCCNC(=O)OCC(CCCC)CCCCCC)CCN(C)CCN(CCC(=O)NCCNC(=O)OCC(CCCC)CCCCCC)CCC(=O)NCCNC(=O)OCC(CCCC)CCCCCC] |
| L0775 | 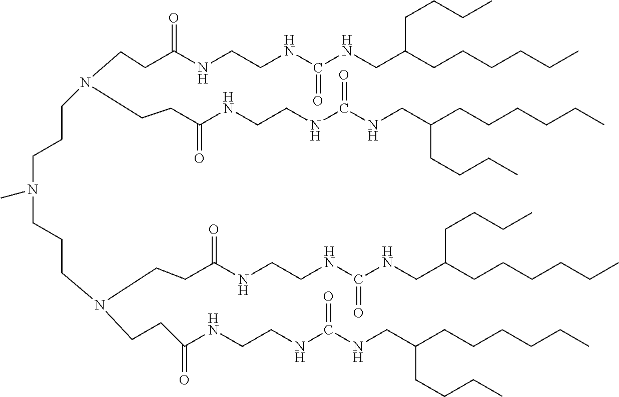 [SMILES: CCCCCCC(CCCC)CNC(=O)NCCNC(=O)CCN(CCCN(C)CCCN(CCC(=O)NCCNC(=O)NCC(CCCC)CCCCCC)CCC(=O)NCCNC(=O)NCC(CCCC)CCCCCC)CCC(=O)NCCNC(=O)NCC(CCCC)CCCCCC] |
| L0776 | 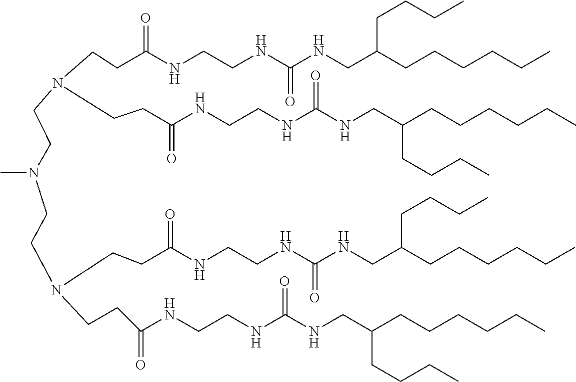 [SMILES: CCCCCCC(CCCC)CNC(=O)NCCNC(=O)CCN(CCC(=O)NCCNC(=O)NCC(CCCC)CCCCCC)CCN(C)CCN(CCC(=O)NCCNC(=O)NCC(CCCC)CCCCCC)CCC(=O)NCCNC(=O)NCC(CCCC)CCCCCC] |
| L0854 | 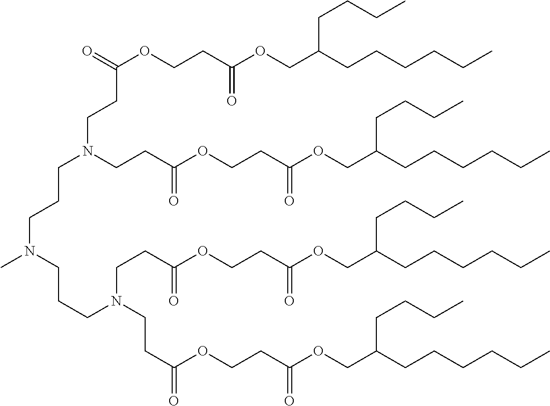 [SMILES: CCCCCCC(CCCC)COC(=O)CCOC(=O)CCN(CCCN(C)CCCN(CCC(=O)OCCC(=O)OCC(CCCC)CCCCCC)CCC(=O)OCCC(=O)OCC(CCCC)CCCCCC)CCC(=O)OCCC(=O)OCC(CCCC)CCCCCC] |
| L0855 | 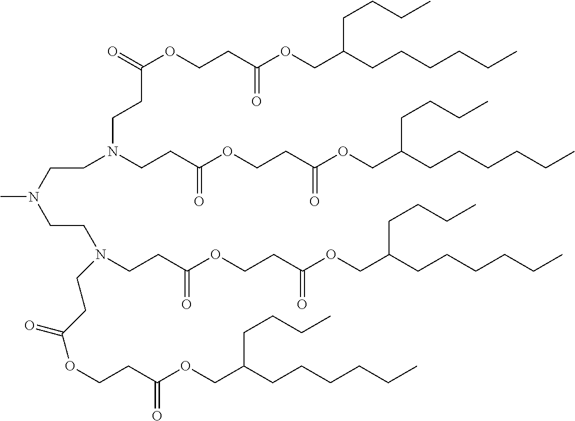 [SMILES: CCCCCCC(CCCC)COC(=O)CCOC(=O)CCN(CCC(=O)OCCC(=O)OCC(CCCC)CCCCCC)CCN(C)CCN(CCC(=O)OCCC(=O)OCC(CCCC)CCCCCC)CCC(=O)OCCC(=O)OCC(CCCC)CCCCCC] |
| L0905 | 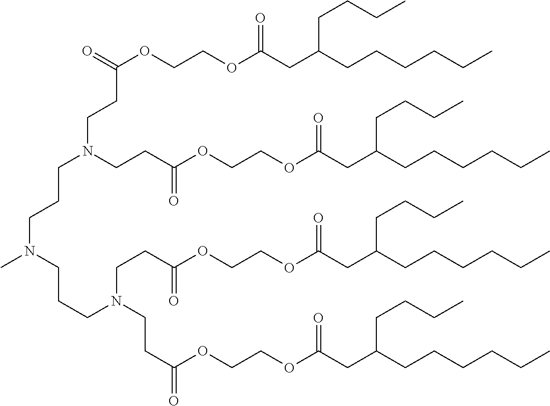 [SMILES: CCCCCCC(CCCC)CC(=O)OCCOC(=O)CCN(CCCN(C)CCCN(CCC(=O)OCCOC(=O)CC(CCCC)CCCCCC)CCC(=O)OCCOC(=O)CC(CCCC)CCCCCC)CCC(=O)OCCOC(=O)CC(CCCC)CCCCCC] |
| L0906 | 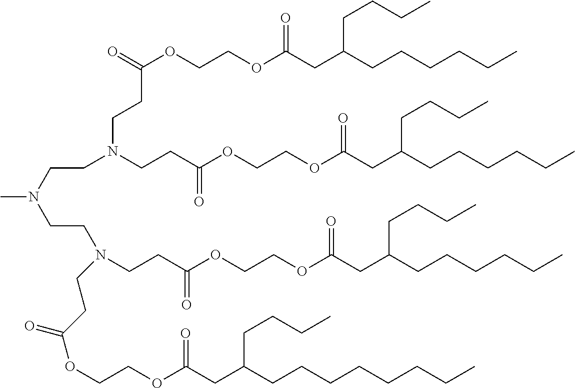 [SMILES: CCCCCCCCC(CCCC)CC(=O)OCCOC(=O)CCN(CCC(=O)OCCOC(=O)CC(CCCC)CCCCCC)CCN(C)CCN(CCC(=O)OCCOC(=O)CC(CCCC)CCCCCC)CCC(=O)OCCOC(=O)CC(CCCC)CCCCCC] |
| L0888 | 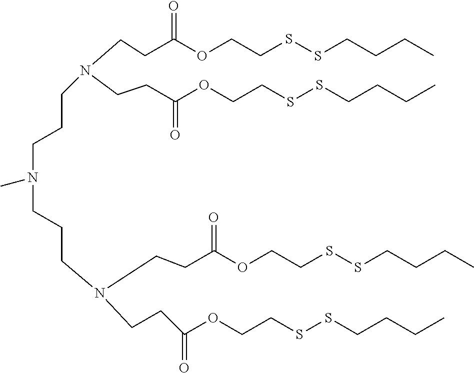 [SMILES: CCCCSSCCOC(=O)CCN(CCCN(C)CCCN(CCC(=O)OCCSSCCCC)CCC(=O)OCCSSCCCC)CCC(=O)OCCSSCCCC] |
| L0889 | 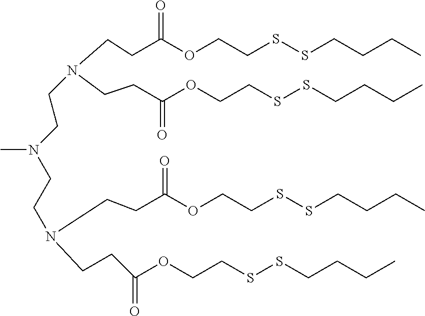 [SMILES: CCCCSSCCOC(=O)CCN(CCC(=O)OCCSSCCCC)CCN(C)CCN(CCC(=O)OCCSSCCCC)CCC(=O)OCCSSCCCC] |
| L0642 | 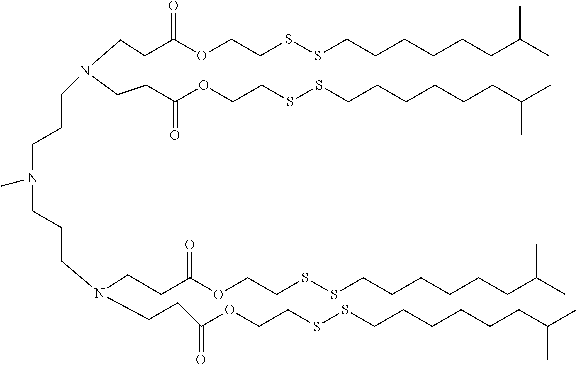 [SMILES: CC(C)CCCCCCSSCCOC(=O)CCN(CCCN(C)CCCN(CCC(=O)OCCSSCCCCCCC(C)C)CCC(=O)OCCSSCCCCCCC(C)C)CCC(=O)OCCSSCCCCCCC(C)C] |
| L0643 | 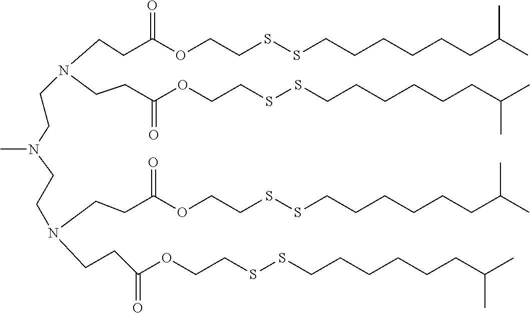 [SMILES: CC(C)CCCCCCSSCCOC(=O)CCN(CCC(=O)OCCSSCCCCCCC(C)C)CCN(C)CCN(CCC(=O)OCCSSCCCCCCC(C)C)CCC(=O)OCCSSCCCCCCC(C)C] |
| L0644 | 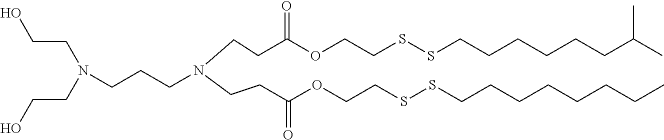 [SMILES: CCCCCCCCSSCCOC(=O)CCN(CCCN(CCO)CCO)CCC(=O)OCCSSCCCCCCC(C)C] |
| L0645 | 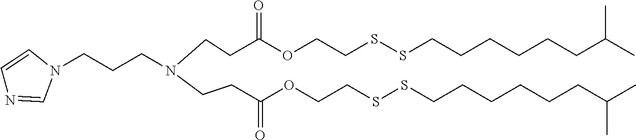 [SMILES: CC(C)CCCCCCSSCCOC(=O)CCN(CCCn1ccnc1)CCC(=O)OCCSSCCCCCCC(C)C] |
| L0893 | 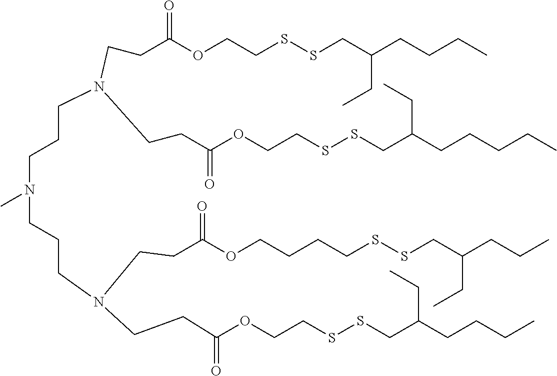 [SMILES: CCCCCC(CC)CSSCCOC(=O)CCN(CCCN(C)CCCN(CCC(=O)OCCCCSSCC(CC)CCC)CCC(=O)OCCSSCC(CC)CCCC)CCC(=O)OCCSSCC(CC)CCCC] |
| L0894 | 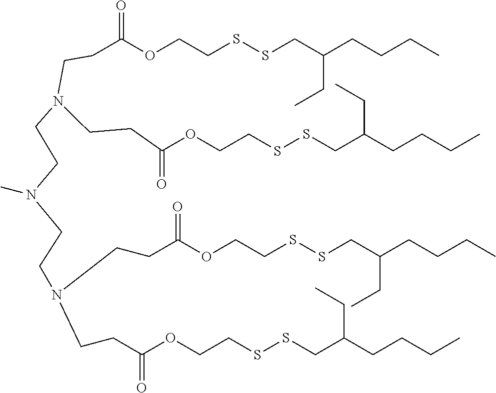 [SMILES: CCCCC(CC)CSSCCOC(=O)CCN(CCC(=O)OCCSSCC(CC)CCCC)CCN(C)CCN(CCC(=O)OCCSSCC(CC)CCCC)CCC(=O)OCCSSCC(CC)CCCC] |
| L0743 | 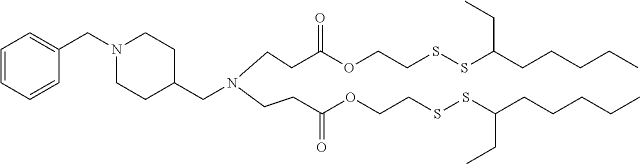 [SMILES: CCCCCC(CC)SSCCOC(=O)CCN(CCC(=O)OCCSSC(CC)CCCCC)CC1CCN(Cc2ccccc2)CC1] |
| L0882 | 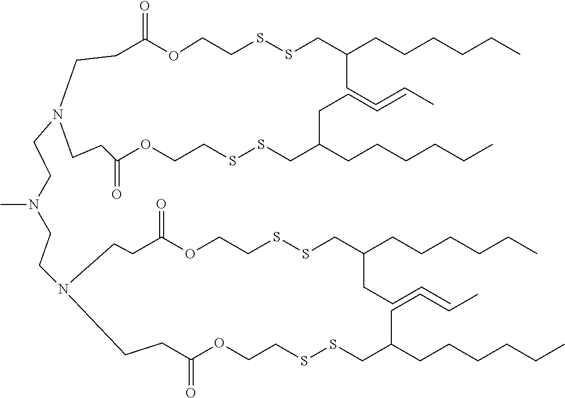 [SMILES: CCCCCCC(CCCC)CSSCCOC(=O)CCN(CCC(=O)OCCSSCC(CCCC)CCCCCC)CCN(C)CCN(CCC(=O)OCCSSCC(CCCC)CCCCCC)CCC(=O)OCCSSCC(CCCC)CCCCCC] |
| L0883 | 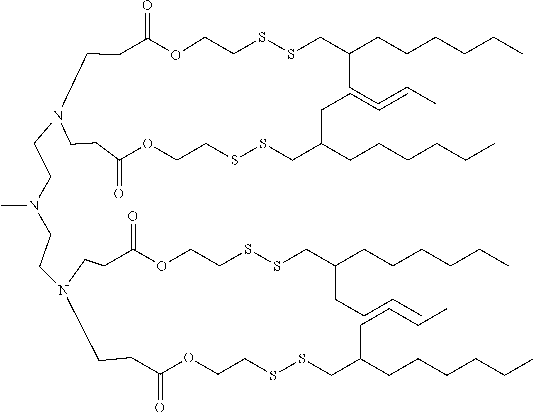 [SMILES: CCCCCCC(CCCC)CSSCCOC(=O)CCN(CCC(=O)OCCSSCC(CCCC)CCCCCC)CCN(C)CCN(CCC(=O)OCCSSCC(CCCC)CCCCCC)CCC(=O)OCCSSCC(CCCC)CCCCCC] |
| L0884 | 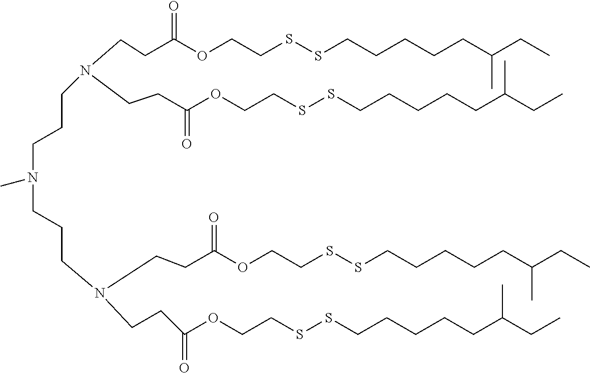 [SMILES: CCC(C)CCCCCSSCCOC(=O)CCN(CCCN(C)CCCN(CCC(=O)OCCSSCCCCCC(C)CC)CCC(=O)OCCSSCCCCCC(C)CC)CCC(=O)OCCSSCCCCCC(C)CC] |
| L0885 | 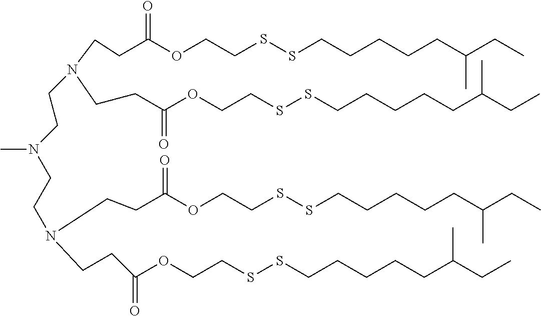 [SMILES: CCC(C)CCCCCSSCCOC(=O)CCN(CCC(=O)OCCSSCCCCCC(C)CC)CCN(C)CCN(CCC(=O)OCCSSCCCCCC(C)CC)CCC(=O)OCCSSCCCCCC(C)CC] |
| L0813 | 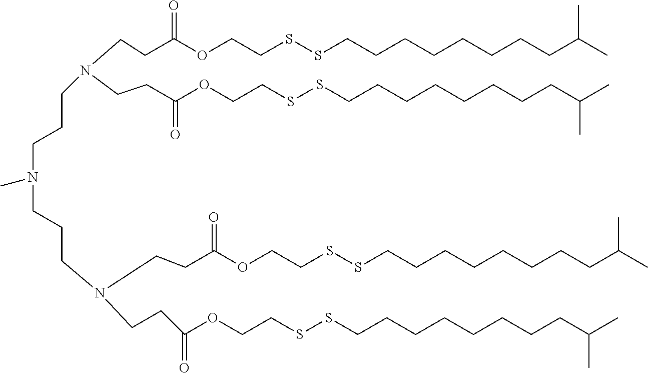 [SMILES: CC(C)CCCCCCCCSSCCOC(=O)CCN(CCCN(C)CCCN(CCC(=O)OCCSSCCCCCCCCC(C)C)CCC(=O)OCCSSCCCCCCCCC(C)C)CCC(=O)OCCSSCCCCCCCCC(C)C] |
| L0814 | 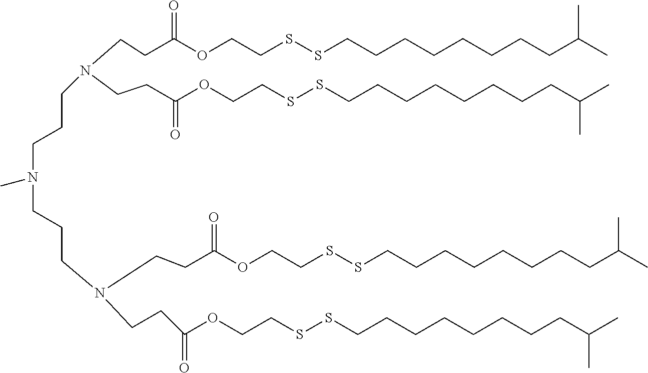 [SMILES: CC(C)CCCCCCCCSSCCOC(=O)CCN(CCCN(C)CCCN(CCC(=O)OCCSSCCCCCCCCC(C)C)CCC(=O)OCCSSCCCCCCCCC(C)C)CCC(=O)OCCSSCCCCCCCCC(C)C] |
| L0886 | 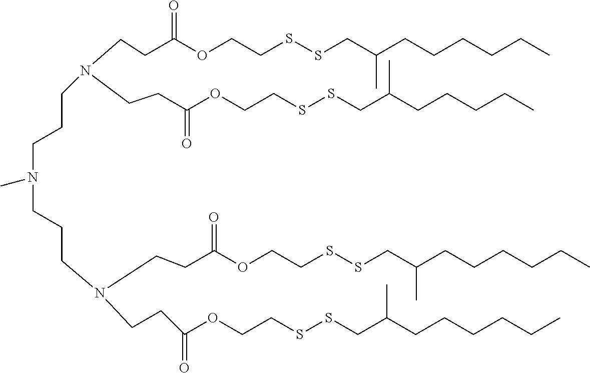 [SMILES: CCCCCCC(C)CSSCCOC(=O)CCN(CCCN(C)CCCN(CCC(=O)OCCSSCC(C)CCCCCC)CCC(=O)OCCSSCC(C)CCCCCC)CCC(=O)OCCSSCC(C)CCCCC] |
| L0887 | 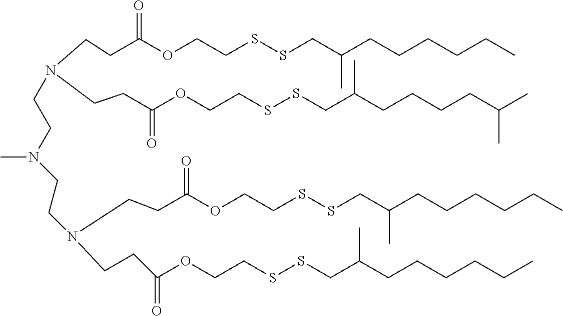 [SMILES: CCCCCCC(C)CSSCCOC(=O)CCN(CCC(=O)OCCSSCC(C)CCCCCC)CCN(C)CCN(CCC(=O)OCCSSCC(C)CCCCCC)CCC(=O)OCCSSCC(C)CCCCC(C)C] |
| L0481 | 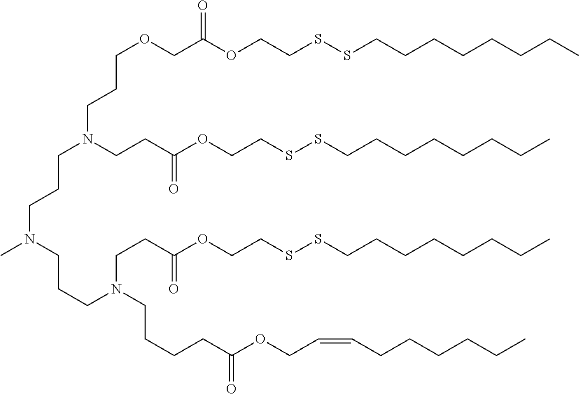 [SMILES: CCCCCC/C=C\COC(=O)CCCCN(CCCN(C)CCCN(CCCOCC(=O)OCCSSCCCCCCCC)CCC(=O)OCCSSCCCCCCCC)CCC(=O)OCCSSCCCCCCCC] |
| L0482 | 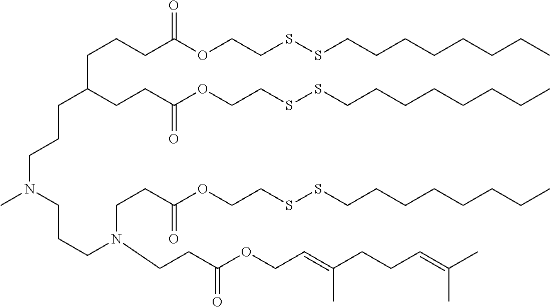 [SMILES: CCCCCCCCSSCCOC(=O)CCCC(CCCN(C)CCCN(CCC(=O)OC/C=C(\C)CCC=C(C)C)CCC(=O)OCCSSCCCCCCCC)CCC(=O)OCCSSCCCCCCCC] |
| L0483 | 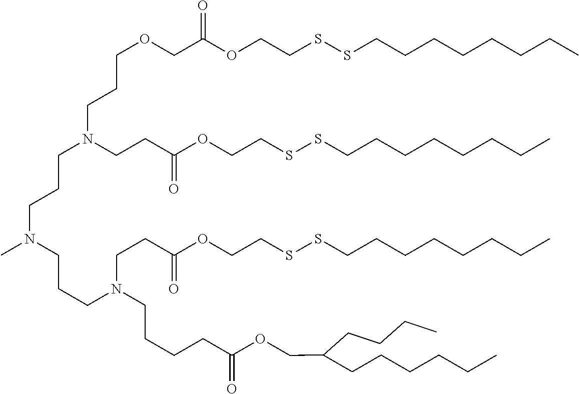 [SMILES: CCCCCCCCSSCCOC(=O)CCN(CCCCC(=O)OCC(CCCC)CCCCCC)CCCN(C)CCCN(CCCOCC(=O)OCCSSCCCCCCCC)CCC(=O)OCCSSCCCCCCCC] |
| L0484 | 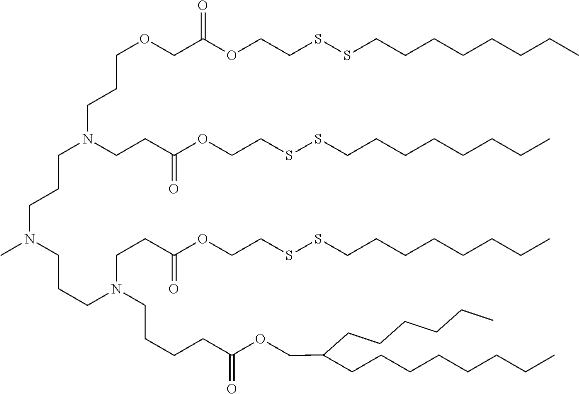 [SMILES: CCCCCCCCSSCCOC(=O)CCN(CCCCC(=O)OCC(CCCCCC)CCCCCCCC)CCCN(C)CCCN(CCCOCC(=O)OCCSSCCCCCCCC)CCC(=O)OCCSSCCCCCCCC] |
| L0486 | 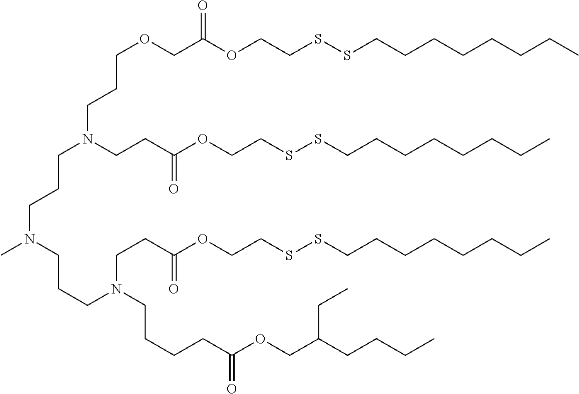 [SMILES: CCCCCCCCSSCCOC(=O)CCN(CCCCC(=O)OCC(CC)CCCC)CCCN(C)CCCN(CCCOCC(=O)OCCSSCCCCCCCC)CCC(=O)OCCSSCCCCCCCC] |
| L0581 | 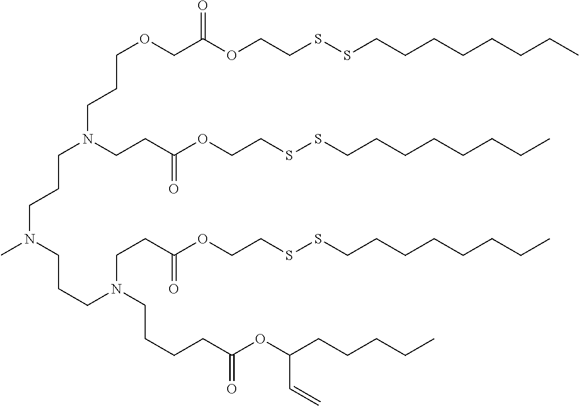 [SMILES: C=CC(CCCCC)OC(=O)CCCCN(CCCN(C)CCCN(CCCOCC(=O)OCCSSCCCCCCCC)CCC(=O)OCCSSCCCCCCCC)CCC(=O)OCCSSCCCCCCCC] |
| L0829 | 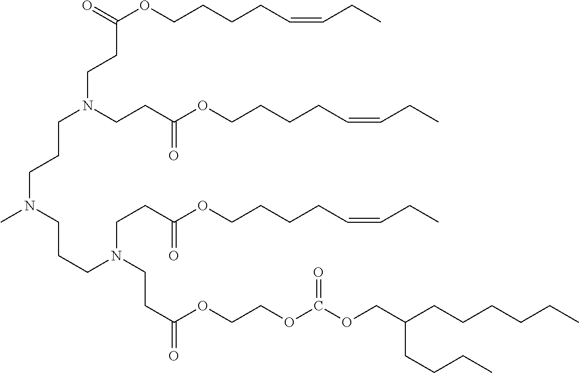 [SMILES: CC/C=C\CCCCOC(=O)CCN(CCCN(C)CCCN(CCC(=O)OCCCC/C=C\CC)CCC(=O)OCCOC(=O)OCC(CCCC)CCCCCC)CCC(=O)OCCCC/C=C\CC] |
| L0830 | 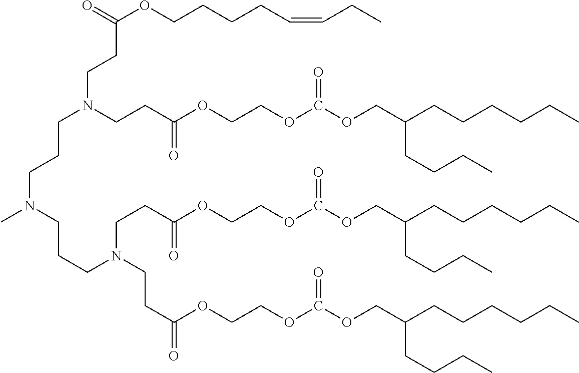 [SMILES: CC/C=C\CCCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0836 | 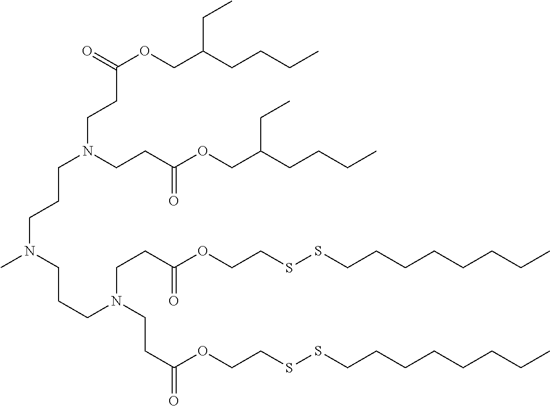 [SMILES: CCCCCCCCSSCCOC(=O)CCN(CCCN(C)CCCN(CCC(=O)OCC(CC)CCCC)CCC(=O)OCC(CC)CCCC)CCC(=O)OCCSSCCCCCCCC] |
| HTX-CH-L0088 | 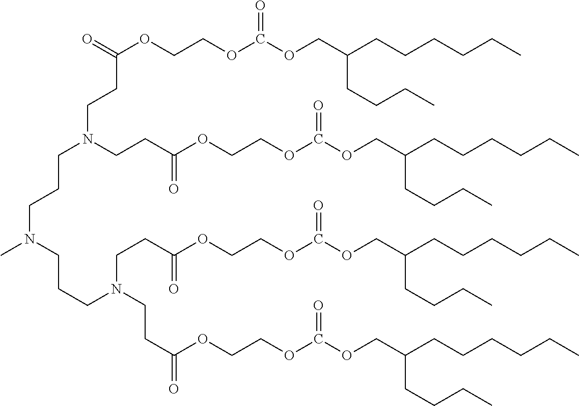 [SMILES: CCCCCCC(CCCC)COC(=O)OCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| HTX-CH-L0093 | 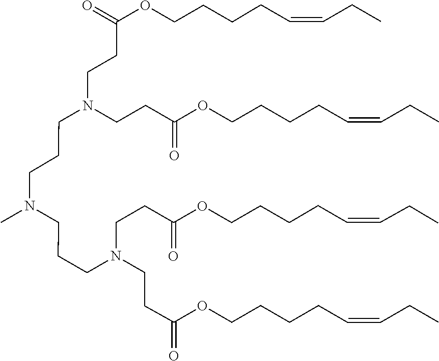 [SMILES: CC/C=C\CCCCOC(=O)CCN(CCCN(C)CCCN(CCC(=O)OCCCC/C=C\CC)CCC(=O)OCCCC/C=C\CC)CCC(=O)OCCCC/C=C\CC] |
| HTX-CH-L0010 | 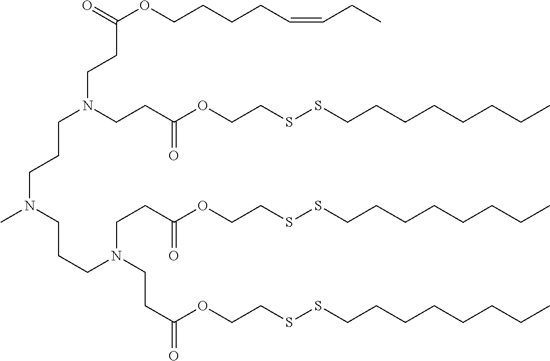 [SMILES: CC/C=C\CCCCOC(=O)CCN(CCCN(C)CCCN(CCC(=O)OCCSSCCCCCCCC)CCC(=O)OCCSSCCCCCCCC)CCC(=O)OCCSSCCCCCCCC] |
| HTX-CH-L0065 | 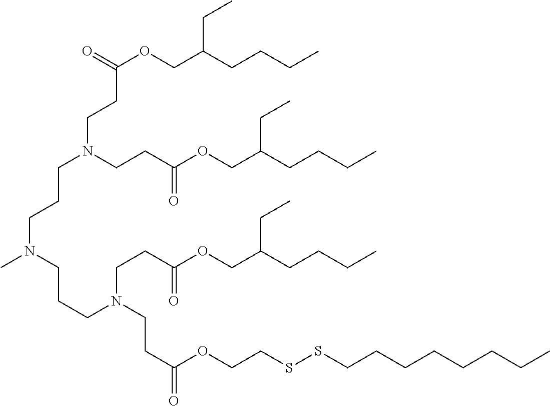 [SMILES: CCCCCCCCSSCCOC(=O)CCN(CCCN(C)CCCN(CCC(=O)OCC(CC)CCCC)CCC(=O)OCC(CC)CCCC)CCC(=O)OCC(CC)CCCC] |
| HTX-CH-L0090 | 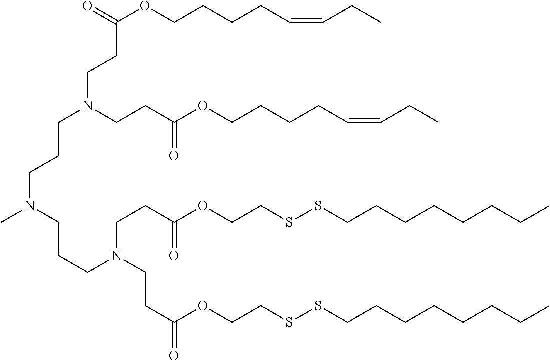 [SMILES: CC/C=C\CCCCOC(=O)CCN(CCCN(C)CCCN(CCC(=O)OCCSSCCCCCCCC)CCC(=O)OCCSSCCCCCCCC)CCC(=O)OCCCC/C=C\CC] |
| HTX-CH-L0095 | 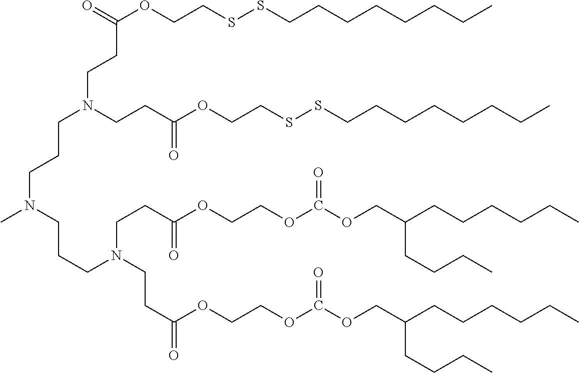 [SMILES: CCCCCCCCSSCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCSSCCCCCCCC] |
| HTX-CH-L0105 | 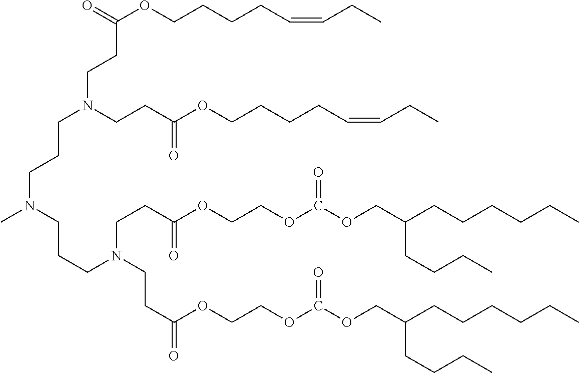 [SMILES: CC/C=C\CCCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCCC/C=C\CC] |
| HTX-CH-L0465 | 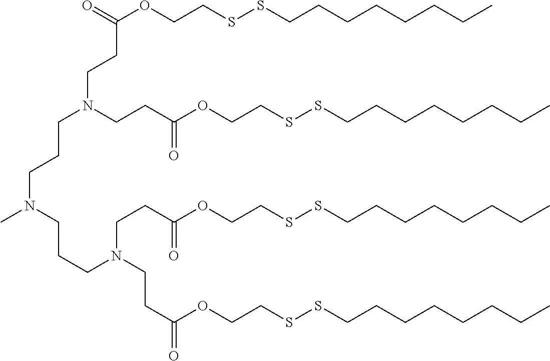 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCCCC] |

In some embodiments, the composition provide herein further comprises a steroid. In some embodiments, the steroid comprises a cholesterol or a cholesterol derivative. In some embodiments, the composition provided herein further comprises a helper lipid. In some embodiments, the helper lipid comprises 1,2-dioleoyl-sn-glycero-3-phosphoethanolamine (DOPE) or 1,2-dioleoyl-sn-glycero-3-phosphocholine (DOPC). In some embodiments, the composition provided herein further comprises a polymer conjugated lipid. In some embodiments, the polymer conjugated lipid comprises 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k). In some embodiments, the lipid composition further comprises a modification comprising a cell-penetrating peptide. In some cases, the cell-penetrating peptide is transactivator of transcription (TAT).

In some embodiments, the lipid composition comprises an ionizable lipid disclosed in this application, a steroid, a helper lipid, and a polymer conjugated lipid.

In some embodiments, the ionizable lipid is present in the lipid composition at a weight percentage from about 10% to about 20%, about 10% to about 30%, about 10% to about 40%, about 10% to about 50%, about 10% to about 60%, about 10% to about 80%, about 10% to about 90%, from about 20% to about 30%, from about 20% to about 30%, from about 20% to about 40%, from about 20% to about 50%, from about 20% to about 60%, from about 20% to about 70%, from about 20% to about 80%, from about 20% to about 90%, from about 30% to about 40%, from about 30% to about 50%, from about 30% to about 60%, from about 30% to about 70%, from about 30% to about 80%, from about 30% to about 90%, from about 40% to about 50%, from about 40% to about 60%, from about 40% to about 70%, from about 40% to about 80%, from about 40% to about 90%, from about 50% to about 60%, from about 50% to about 70%, from about 50% to about 80%, from about 50% to about 90%, from about 60% to about 70%, from about 60% to about 80%, from about 60% to about 90%, from about 70% to about 80%, from about 70% to about 90%, or from about 80% to about 90%.

In some embodiments, the helper lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the steroid is present in the lipid composition at a weight percentage from about 10% to about 20%, from about 10% to about 30%, from about 10% to about 40%, from about 20% to about 30%, from about 20% to about 40%, or from about 30% to about 40%.

In some embodiments, the polymer conjugated lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the weight ratio of the ionizable lipid/steroid/helper lipid/polymer conjugated lipid is about 14/4/1/1, about 15/4/1/1, about 16/4/1/1, about 17/4/1/1, about 18/4/1/1, about 19/4/1/1, about 20/4/1/1, about 14/4/2/1, about 15/4/2/1, about 16/4/2/1, about 16.4/4/2/1, about 16.6/4/2/1, about 16.8/4/2/1, about 17/4/2/1, about 18/4/2/1, about 19/4/2/1, or about 20/4/2/1. In some embodiments, the weight ratio of the ionizable lipid/steroid/helper lipid/polymer conjugated lipid is about 16.4/4/2/1 or 16.6/4/2/1.

In some embodiments, the lipid composition comprises an ionizable lipid disclosed in this application, a steroid and a helper lipid. In some embodiments, the ionizable lipid is present in the lipid composition at a weight percentage of from about 30% to about 90%, from about 40% to about 90%, from about 50% to about 90%, from about 60% to about 90%, from about 70% to about 90%, from about 50% to about 80%, from about 60% to about 80%, or from about 70% to about 80%.

In some embodiments, the helper lipid is present in the lipid composition at a weight percentage of from about 5% to about 40%, from about 5% to about 30%, from about 5% to about 20%, from about 5% to about 15%, from about 5% to about 10%, from about 7.5% to about 40%, from about 7.5% to about 30%, from about 7.5% to about 20%, from about 7.5% to about 15%, from about 7.5% to about 10%, from about 10% to about 40%, from about 10% to about 30%, from about 10% to about 20%, from about 10% to about 1⁷0.5%, or from about 10% to about 15%. In some embodiments, the helper lipid is present in the lipid composition at a weight percentage of from about 12% to about 14%.

In some embodiments, the steroid is present in the lipid composition at a weight percentage of from about 5% to about 40%, from about 5% to about 30%, from about 5% to about 20%, from about 5% to about 15%, from about 5% to about 10%, from about 7.5% to about 40%, from about 7.5% to about 30%, from about 7.5% to about 20%, from about 7.5% to about 15%, from about 7.5% to about 10%, from about 10% to about 40%, from about 10% to about 30%, from about 10% to about 20%, from about 10% to about 17.5%, from about 15% to about 30%, from about 15% to about 25%, from about 15% to about 22.5%, from about 15% to about 20%, or from about 15% to about 17.5%. In some embodiments, the steroid is present in the lipid composition at a weight percentage of from about 16.5% to about 17.5%.

In some embodiments, the weight ratio of the ionizable lipid/steroid/helper lipid is about 1/1/1, 2/1/1, about 3/1/1, about 4/1/1, about 5/1/1, about 6/1/1, about 2/2/1, about 3/2/1, about 4/2/1, about 5/2/1, about 20/4/3, about 17/4/3, about 16/4/3, about 8/2/1, or about 6/2/1. In some embodiments, the weight ratio of the ionizable lipid/steroid/helper lipid is about 16.6/4/3.

In some embodiments, the lipid composition further comprises an excipient. The excipient can comprise EC-16, (2-hydroxypropyl)-p-cyclodextrin ((HP-3-CD), stearic acid, Perfluoroundecanoic, Saponin, Mannitol, Borneol, Amikacin-EC16, Kanamycin-EC16, Neomycin-EC16, or Bile salts. In some embodiments, the excipient is present in the composition at a weight percentage from about 5% to about 60%. In some embodiments, the excipient is present in the composition at a weight percentage from about 1% to about 70%, from about 5% to about 60%, from about 5% to about 50%, from about 5% to about 40%, from about 5% to about 30%, from about 10% to about 50%, from about 10% to about 40%, from about 10% to about 30%, or from about 10% to about 20%.

In some embodiments, the pharmaceutical agent comprises a polynucleotide, an oligonucleotide, a polypeptide, an oligopeptide, a small molecule compound, or any combination thereof. In some embodiments, the polynucleotide is a messenger ribonucleic acid (mRNA).

In some embodiments, the pharmaceutical agent comprises a polynucleotide that encodes or is configured to regulate a target gene or a gene product thereof.

In some embodiments, the pharmaceutical agent comprises (a) a gene modulating moiety configured to specifically bind at least a portion of a target gene or a gene product thereof, or (b) a polynucleotide that encodes a gene modulating moiety configured to specifically bind at least a portion of a target gene or a gene product thereof.

In some embodiments, the gene modulating moiety comprises a guide nucleic acid configured to complex with at least a portion of the target gene or the gene product thereof, or a polynucleotide sequence that encodes the guide nucleic acid.

In some embodiments, the gene modulating moiety comprises a heterologous endonuclease or a polynucleotide comprising a sequence that encodes the heterologous endonuclease. In some embodiments, the heterologous endonuclease comprises a clustered regularly interspaced short palindromic repeats (CRISPR)-associated (Cas) nuclease. In some embodiments, the polynucleotide comprising a sequence that encodes the heterologous endonuclease.

In some embodiments, the pharmaceutical agent (or therapeutic agent) comprises mRNA encoding at least one therapeutic peptide for immunotherapy. In some embodiments, the pharmaceutical agent (or therapeutic agent) is suitable for cancer immunotherapy.

In some embodiments, the pharmaceutical agent is assembled in the lipid composition at a weight ratio of the pharmaceutical agent/lipid composition of from about 1:200 to about 1:100, from about 1:200 to about 1:50, from about 1:200 to about 1:40, from about 1:200 to about 1:30, from about 1:200 to about 1:20, from about 1:200 to about 1:10, from about 1:200 to about 1:5, from about 1:200 to about 1:1, from about 1:100 to about 1:50, from about 1:100 to about 1:40, from about 1:100 to about 1:25, from about 1:100 to about 1:20, from about 1:100 to about 1:15, from about 1:100 to about 1:10, from about 1:100 to about 1:5, from about 1:100 to about 1:1, from about 1:50 to about 1:30, from about 1:50 to about 1:25, from about 1:50 to about 1:20, from about 1:40 to about 1:30, from about 1:40 to about 1:25, from about 1:40 to about 1:20, or from about 1:30 to 1:20. In some embodiments, the pharmaceutical agent is assembled in the lipid composition at a weight ratio of the pharmaceutical agent/lipid composition of 1:25.

In some embodiments, the target gene or the gene product thereof is specific to or primarily found in a target organ (e.g., liver, spleen, or lymph nodes) or a target cell (e.g., a immune cell provided herein; e.g., a T cell) of a subject.

In some embodiments, the target gene or the gene product thereof is associated with a disease or disorder of the target organ or the target cell.

In some embodiments, the gene modulating moiety is configured to provide a modified expression profile of the target gene or the gene product thereof in a target organ (e.g., liver, spleen, lymph nodes) or a target cell (e.g., an immune cell provided herein; e.g., a T cell) of a subject.

In some embodiments, the composition is formulated for systemic or local administration. In some embodiments, the composition is formulated for intravenous administration. In some embodiments, the composition is formulated for intramuscular administration.

### Additional Lipids

In some embodiments, the lipid composition further comprises an additional lipid comprising a steroid or a steroid derivative, a PEG lipid, and a helper lipid (e.g., phospholipids or other zwitterionic lipids).

In some embodiments, the lipid composition further comprises a helper lipid. In some embodiments, the helper lipid comprises a lipid that contributes to the stability or delivery efficiency of the lipid compositions. In some embodiments, the helper lipid comprises a zwitterionic lipid. In some embodiments, the helper lipid comprises a phospholipid. In some embodiments, the phospholipid may contain one or two long chain (e.g., C6-C24) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge. In some embodiments of the lipid compositions, the phospholipid is not an ethylphosphocholine. In some embodiments, the helper lipid can comprise 1,2-dioleoyl-sn-glycero-3-phosphoethanolamine (DOPE) or 1,2-dioleoyl-sn-glycero-3-phosphocholine (DOPC).

In some embodiments, the compositions may further comprise a molar percentage of the phospholipid to the total lipid composition from about 5 to about 30.

In some embodiments, the helper lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the lipid composition comprises the phospholipid at a molar percentage from about 8% to about 23%. In some embodiments, the lipid composition comprises the phospholipid at a molar percentage from about 10% to about 20%. In some embodiments, the lipid composition comprises the phospholipid at a molar percentage from about 15% to about 20%. In some embodiments, the lipid composition comprises the phospholipid at a molar percentage from about 8% to about 15%. In some embodiments, the lipid composition comprises the phospholipid at a molar percentage from about 10% to about 15%. In some embodiments, the lipid composition comprises the phospholipid at a molar percentage from about 12% to about 18%. In some embodiments, the lipid composition comprises the phospholipid at a molar percentage of at least about 8%, at least about 10%, at least about 12%, at least about 15%, at least about 18%, at least about 20%, or at least about 23%. In some embodiments, the lipid composition comprises the phospholipid at a molar percentage of at most about 8%, at most about 10%, at most about 12%, at most about 15%, at most about 18%, at most about 20%, or at most about 23%.

In some embodiments, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

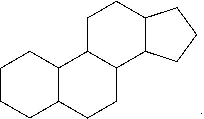 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

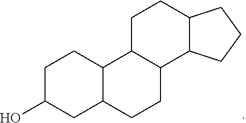 [SMILES: OC1CCC2C(CCC3C4CCCC4CCC23)C1]

In some embodiments, the steroid or steroid derivative is a cholestane or cholestane derivative. In a cholestane, the ring structure is further defined by the formula:

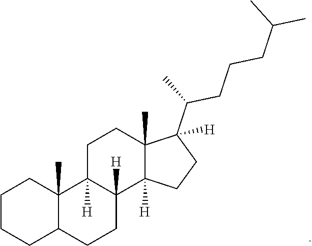

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholesterol and a sterol or a derivative thereof.

In some embodiments, the compositions may further comprise a molar percentage of the steroid to the total lipid composition from about 20 to about 60. In some embodiments, the steroid is present in the lipid composition at a weight percentage from about 10% to about 20%, from about 10% to about 30%, from about 10% to about 40%, from about 20% to about 30%, from about 20% to about 40%, or from about 30% to about 40%.

In some embodiments, the lipid composition comprises the steroid or steroid derivative at a molar percentage from about 15% to about 46%. In some embodiments, the lipid composition comprises the steroid or steroid derivative at a molar percentage from about 20% to about 40%. In some embodiments, the lipid composition comprises the steroid or steroid derivative at a molar percentage from about 25% to about 35%. In some embodiments, the lipid composition comprises the steroid or steroid derivative at a molar percentage from about 30% to about 40%. In some embodiments, the lipid composition comprises the steroid or steroid derivative at a molar percentage from about 20% to about 30%. In some embodiments, the lipid composition comprises the steroid or steroid derivative at a molar percentage of at least about 15%, at least about 20%, at least about 25%, at least about 30%, at least about 35%, at least about 40%, at least about 45%, or at least about 46%. In some embodiments, the lipid composition comprises the steroid or steroid derivative at a molar percentage of at most about 15%, at most about 20%, at most about 25%, at most about 30%, at most about 35%, at most about 40%, at most about 45%, or at most about 46%.

In some embodiments, the lipid composition further comprises a polymer conjugated lipid. In some embodiments, the polymer conjugated lipid is a PEG lipid. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present application are taught by U.S. Pat. No. 5,820,873, WO 2010/141069, or U.S. Pat. No. 8,450,298, which is incorporated herein by reference.

In some embodiments, the PEG lipid has a structural formula:

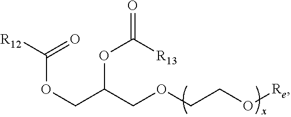

X, wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments, the PEG lipid has a structural formula:

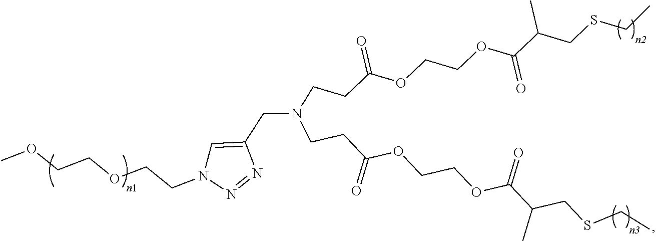

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments, the polymer conjugated lipid comprises 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k).

In some embodiments, the lipid composition comprises the polymer-conjugated lipid at a molar percentage from about 0.5% to about 20%. In some embodiments, the lipid composition comprises the polymer-conjugated lipid at a molar percentage from about 1% to about 8%. In some embodiments, the lipid composition comprises the polymer-conjugated lipid at a molar percentage from about 2% to about 7%. In some embodiments, the lipid composition comprises the polymer-conjugated lipid at a molar percentage from about 3% to about 5%. In some embodiments, the lipid composition comprises the polymer-conjugated lipid at a molar percentage from about 5% to about 10%. In some embodiments, the lipid composition comprises the polymer-conjugated lipid at a molar percentage of at least about 0.5%, at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, or at least about 10%. In some embodiments, the lipid composition comprises the polymer-conjugated lipid at a molar percentage of at most about 0.5%, at most about 1%, at most about 1.5%, at most about 2%, at most about 2.5%, at most about 3%, at most about 3.5%, at most about 4%, at most about 4.5%, at most about 5%, at most about 5.5%, at most about 6%, at most about 6.5%, at most about 7%, at most about 7.5%, at most about 8%, at most about 8.5%, at most about 9%, at most about 9.5%, at most about 10%, at most about 15%, or at most 20%.

In some embodiments, the polymer conjugated lipid is present in the lipid composition at a weight percentage from about 1% to about 5%, from about 1% to about 10%, from about 1% to about 20%, from about 5% to about 10%, from about 5% to about 20%, or from about 10% to about 20%.

### Therapeutic Payloads

Provided herein are lipid nanoparticle compositions comprising a therapeutic payload (e.g., a pharmaceutical agent or a therapeutic agent). The lipids and lipid compositions can be used for delivering a plurality of therapeutic payloads. In some embodiments, the therapeutic payload can comprise a peptide, a nucleic acid molecule, a small molecule drug, or a combination thereof. In some embodiments, the therapeutic payload comprises a therapeutic peptide. In some embodiments, the therapeutic payload comprises a nucleic acid molecule. The nucleic acid molecule can comprise an engineered DNA, RNA (e.g., mRNA, sgRNA), a hybrid nucleic acid or a combination thereof. In some embodiments, the nucleic acid molecule can encode a therapeutic peptide.

In some embodiments, the lipid nanoparticle (LNP) compositions comprises a therapeutic peptide or nucleic acid molecules (e.g., mRNA or DNA) encoding a therapeutic peptide. In some embodiments, the LNP comprises an mRNA encoding a therapeutic peptide. The therapeutic peptides may be suitable for immunotherapy. In some embodiments, the therapeutic peptide comprises a secretary peptide. In some embodiments, the therapeutic peptide comprises a transmembrane peptide. In some embodiments, the therapeutic peptide comprises an intracellular peptide.

In some cases, the therapeutic peptides for immunotherapy comprise heterologous immune receptors (e.g., antigen receptors), antigen-binding peptides (e.g., antibodies or fragments thereof), or immunomodulators. In some cases, a heterologous immune receptor comprises an antigen receptor, e.g., a chimeric antigen receptor (CAR) on immune cells (e.g., T cells). CARs can be designed for cell-based immunotherapy, for example, for CAR-T or CAR-pool therapy. The antigen-binding peptides of the present disclosure comprise secreted therapeutic peptides that can bind to an antigen (e.g., a cancer antigen). In some cases, the antigen-binding peptides can be multi-specific and bind to more than one antigen. In some embodiments, the multi-specific antigen-binding peptide can bind to an immune cell marker (e.g., CD3 on a T-cell) and a tumor antigen.

In some cases, the therapeutic peptides may further comprise a protein tag (e.g., a 6× His tag). In some cases, the protein tag can facilitate identification or purification of the therapeutic peptide.

In some cases, the LNPs of the present disclosure comprises one or more therapeutic peptides. In some embodiments, the LNP comprises two or more therapeutic peptides. In some embodiments, the LNP comprises 1, 2, 3, 4, 5, 6 or more therapeutic peptides.

In some embodiments, the LNP comprises one or more nucleic acid molecules (e.g., mRNA or DNA) encoding one or more therapeutic peptides for immunotherapy. In some cases, the LNPs comprises mRNA(s) encoding 1, 2, 3, 4, 5, 6, or more therapeutic peptides. In some embodiments, the LNP comprises a therapeutic peptide and a nucleic acid molecule encoding a therapeutic peptide.

In some embodiments, the therapeutic peptides may be an antigen-binding peptide. In some embodiments, the therapeutic peptides may be a multispecific antigen-binding peptide. In some cases, a multispecific antigen-binding peptide comprises a first domain that binds to an antigen expressed by an immune cell and a second domain that binds to an antigen expressed by a target cell (e.g., a tumor cell). The multispecific antigen-binding peptide can bind to an antigen expressed by any one of the immune cells provided herein.

In some embodiments, the therapeutic peptide comprises a chimeric antigen receptor derived from a neutralizing antibody comprising an antigen-binding fragment, a transmembrane domain, and an intracellular signaling domain described herein. The term “signaling domain” refers to the functional portion of a protein which acts by transmitting information within the cell to regulate cellular activity via defined signaling pathways by generating second messengers or functioning as effectors by responding to such messengers.

An “intracellular signaling domain,” as the term is used herein, refers to an intracellular portion of a molecule. The intracellular signaling domain generates a signal that promotes an immune effector function of the CAR containing cell, e.g., a CAR-T cell. Examples of immune effector function, e.g., in a CAR-T cell, include cytolytic activity and helper activity, including the secretion of cytokines. In some cases, the intracellular signaling domain can comprise a primary intracellular signaling domain. Exemplary primary intracellular signaling domains include those derived from the molecules responsible for primary stimulation, or antigen dependent simulation. In some cases, the intracellular signaling domain can comprise a costimulatory intracellular domain. Exemplary costimulatory intracellular signaling domains include those derived from molecules responsible for costimulatory signals, or antigen independent stimulation. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP 12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that can be used for an efficient immune response. Costimulatory molecules include, but are not limited to, an MHC class I molecule, BTLA and a Toll ligand receptor, as well as OX40, CD2, CD27, CD28, CD5, ICAM-1, LFA-1 (CD11a/CD18) and 4-1BB (CD137).

A costimulatory intracellular signaling domain can be derived from the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like.

The intracellular signaling domain can comprise the entire intracellular portion, or the entire native intracellular signaling domain, of the molecule from which it is derived, or a functional fragment thereof.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR is used. In some cases, the transmembrane domain can be selected or modified by amino acid substitution to avoid binding of such domains to the transmembrane domains of the same or different surface membrane proteins, e.g., to minimize interactions with other members of the receptor complex. In some cases, the transmembrane domain is capable of homodimerization with another CAR on the CAR-T cell surface. In some cases, the amino acid sequence of the transmembrane domain can be modified or substituted so as to minimize interactions with the binding domains of the native binding partner present in the same CAR-T cell.

The transmembrane domain can be derived either from a natural or from a recombinant source. Where the source is natural, the domain may be derived from any membrane-bound or transmembrane protein. In some cases, the transmembrane domain is capable of signaling to the intracellular domain(s) whenever the CAR has bound to a target. In some cases, the transmembrane domain can include at least the transmembrane region(s) of e.g., the alpha, beta, or zeta chain of the T-cell receptor, CD28, CD3 epsilon, CD45, CD4, CD5, CD8, CD9, CD16, CD22, CD33, CD37, CD64, CD80, CD86, CD134, CD137, CD154.

In some cases, the transmembrane domain can be attached to the extracellular region of the CAR, e.g., the antigen-binding domain of the CAR, via a hinge or a spacer, e.g., a hinge from a human protein. In some cases, the hinge can be a human Ig (immunoglobulin) hinge, e.g., an IgG4 hinge, or a CD8a hinge. In one aspect, the hinge or spacer comprises an IgG4 hinge.

The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. The term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the present disclosure include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal can also be involved. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic signaling domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use herein include those of TCR zeta, FcR gamma, FcR beta, CD3 gamma, CD3 delta, CD3 epsilon, CD5, CD22, CD79a, CD79b, and CD66d. In some cases, a CAR comprises an intracellular signaling domain, e.g., a primary signaling domain, of CD3-zeta. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

A costimulatory molecule is a cell surface molecule other than an antigen receptor or its ligands that can play a role for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CAR-T cells in vitro and augments human T cell persistence and antitumor activity in vivo (Song et al. Blood. 2012; 119(3):696-706).

A short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In some cases, the two or more, e.g., 2, 3, 4, 5, or more, costimulatory signaling domains, are separated by a linker molecule, e.g., a linker molecule described herein. In some cases, the intracellular signaling domain comprises two costimulatory signaling domains. In some cases, the linker molecule is a glycine residue. In some embodiments, the linker is an alanine residue.

In some embodiments, the therapeutic peptide can comprise an immunomodulator or a nucleic acid molecule (e.g., mRNA) encoding an immunomodulator.

In some cases, an immunomodulator comprises a cytokine, cytokine receptor, chemokine, chemokine receptor, immune co-receptor, or immune co-receptor ligand. Expression of the immunomodulator can be driven by an expression regulatory region disclosed herein.

In some cases, the therapeutic peptide comprises a cytokine or a functional fragment thereof, for example, G-CSF, GITRL, GM-CSF, IFN-α, IFN-β, IFN-γ, IL-lRA, IL-1α, IL-1β, IL-2, IL-3, IL-4, IL-5, IL-6, IL-7, IL-9, IL-10, IL-11, IL-12, IL-13, IL-14, IL-15, IL-16, IL-17, IL-18, IL-20, IL-23, LIF, LIGHT, LT-P, M-CSF, MSP, OSM, OX40L, SCF, TALL-1, TGF-β, TGF-β1, TGF-β2, TGF-β3, TNF-α, TNF-β, TRAIL, TRANCE, or TWEAK.

In some cases, the therapeutic peptide comprises a cytokine receptor or a functional fragment thereof, for example, a common gamma chain receptor, a common beta chain receptor, an interferon receptor, a TNF family receptor, a TGF-B receptor, Apo3, CD114, CD115, CD116, CD117, CD118, CD120, CD120a, CD120b, CD121, CD121a, CD121b, CD122, CD123, CD124, CD126, CD127, CD130, CD131, CD132, CD212, CD213, CD213a1, CD213a13, CD213a2, CD25, CD27, CD30, CD4, CD40, CD95 (Fas), CDwl19, CDwl21b, CDwl25, CDwl31, CDwl36, CDwl37 (41BB), CDw210, CDw217, GITR, HVEM, IL-11R, IL-11Ra, IL-14R, IL-15R, IL-15Ra, IL-18R, IL-18Rα, IL-18Rβ, IL-20R, IL-20Rα, IL-20Rβ, IL-9R, LIFR, LTβR, OPG, OSMR, OX40, RANK, TACI, TGF-βR1, TGF-βR2, TGF-βR3, TRAILR1, TRAILR2, TRAILR3, or TRAILR4.

In some cases, the therapeutic peptide for immunotherapy comprises a chemokine or a functional fragment thereof, for example, ACT-2, AMAC-a, ATAC, ATAC, BLC, CCL1, CCL11, CCL13, CCL14, CCL15, CCL16, CCL17, CCL18, CCL19, CCL2, CCL20, CCL21, CCL22, CCL23, CCL24, CCL25, CCL26, CCL27, CCL3, CCL4, CCL5, CCL7, CCL8, CKb-6, CKb-8, CTACK, CX3CL1, CXCL1, CXCL10, CXCL11, CXCL12, CXCL13, CXCL14, CXCL2, CXCL3, CXCL4, CXCL5, CXCL6, CXCL7, CXCL8, CXCL9, DC-CK1, ELC, ENA-78, eotaxin, eotaxin-2, eotaxin-3, Eskine, exodus-1, exodus-2, exodus-3, fractalkine, GCP-2, GROa, GROb, GROg, HCC-1, HCC-2, HCC-4, I-309, IL-8, ILC, IP-10, I-TAC, LAG-1, LARC, LCC-1, LD78a, LEC, Lkn-1, LMC, lymphoactin, lymphoactin b, MCAF, MCP-1, MCP-2, MCP-3, MCP-4, MDC, MDNCF, MGSA-a, MGSA-b, MGSA-g, Mig, MIP-1d, MIP-1a, MIP-1, MIP-2a, MIP-2b, MIP-3, MIP-3a, MIP-30, MIP-4, MIP-4a, MIP-5, MPIF-1, MPIF-2, NAF, NAP-1, NAP-2, oncostatin, PARC, PF4, PPBP, RANTES, SCM-1a, SCM-1b, SDF-1α/β, SLC, STCP-1, TARC, TECK, XCL1, or XCL2.

In some cases, the therapeutic peptide for immunotherapy comprises a chemokine receptor or a functional fragment thereof, for example, CCR1, CCR2, CCR3, CCR4, CCR5, CCR6, CCR7, CCR8, CCR9, CCR10, CX3CR1, CXCR1, CXCR2, CXCR3, CXCR4, CXCR5, XCR1, or XCR1.

In some cases, the therapeutic peptide for immunotherapy comprises costimulatory immune receptor, or a functional fragment thereof, for example, CD28, 2B4 (CD244, SLAMF4), 4-1BB (CD137), CD2 (LFA2, OX34), CD21, CD226 (DNAM1), CD27 (TNFRSF7), CD30 (TNFRSF8), CD4, CD40, CD8, CD84 (SLAMF5), CRACC (CD319,BLAME), CRTAM (CD355), DcR3, DR3 (TNFRSF25), GITR (CD357), HVEM (CD270), ICOS (CD278), LIGHT, LTOR (TNFRSF3), Ly108 (NTBA,CD352,SLAMF6), Ly9 (CD229,SLAMF3), OX40 (CD134), SLAM (CD150,SLAMF1), TIM1 (HAVCRI,KIM1), or TIM2.

In some cases, therapeutic peptide for immunotherapy comprises an activating NK receptor, or a functional fragment thereof, for example, CD100 (SEMA4D), CD16 (FcgRIIIA), CD160 (BY55), CD244 (2B4, SLAMF4), CD27, CD94− NKG2C, CD94− NKG2E, CD94-NKG2H, CD96, CRTAM, DAP12, DNAM1 (CD226), KIR2DL4, KIR2DS1, KIR2DS2, KIR2DS3, KIR2DS4, KIR2DS5, KIR3DS1, Ly49, NCR, NKG2D (KLRK1, CD314), NKp30 (NCR3), NKp44 (NCR2), NKp46 (NCR1), NKp80 (KLRF1, CLEC5C), NTB-A (SLAMF6), PSGL1, or SLAMF7 (CRACC, CS1, CD319).

In some cases, the therapeutic peptide for immunotherapy comprises an Fcγ receptor (FcγR), an Fcε receptor (FcεR), an Fcα receptor (FcαR), an Fcμ receptor (FcμR), neonatal Fc receptor (FcRn), CD4, CD5, CD8, CD21, CD22, CD27, CD28, CD32, CD40, CD45, CD66d, CD79a, CD79b, CD80, CD86, CD278 (ICOS), CD247ζ, CD247η, 41BB, DAP10, DAP12, FYN, LAT, Lck, MAPK, MHC complex, NFAT, NF-κB, PLC-γ, iC3b, C3dg, C3d, Zap70, MyD88, a functional fragment thereof, or a combination thereof.

In some cases, the therapeutic peptide for immunotherapy comprises a domain that is, is derived from, interacts with, increases expression of, or activates a transcription factor, such as, for example, E2A, Pax5, EBF, PU.1, Ikaros, GATA3, Th-POK, Tbet, Bc16, NF-κB, NFAT, AP-1, NFAT, STAT1, STAT2, STAT3, STAT4, STAT5, STAT5A, STAT5B, STAT6, STAT7, IRF1, IRF2, IRF3, IRF4, IRF5, IRF6, IRF7, IRF8, IRF9, AP-1, Eomes, FoxP3, Id2, PLZF, ROR-gamma-T, TCF7, ThPOK, or any combination thereof.

### Nucleic Acids

The LNPs provided herein may comprise nucleic acid molecules encoding a therapeutic peptide for immunotherapy. The nucleic acid may comprise an RNA, a DNA, a DNA/RNA hybrid, and/or comprises at least one of a nucleic acid analog, a chemically modified nucleic acid, or a chimera composed of two or more nucleic acids or nucleic acid analogs. In some embodiments, the LNP may comprise an mRNA, wherein the mRNA is encapsulated within the LNP.

In some cases, the nucleic acid molecules encapsulated by the lipid composition (LNP) further comprises regulatory sequences that can facilitate and/or promote expression of the therapeutic peptide for immunotherapy. The regulatory sequences can comprise a 5′ cap, a 5′ untranslated region, a promoter, a signal peptide sequence, a 3′ untranslated region, and a poly-A tail. In some cases, the regulatory sequences comprise an enhancer (e.g., CMV enhancer) to further enhance expression of the therapeutic peptide.

In some cases, the nucleic acid molecules may encode a signal peptide and the signal peptide can be cleaved during post-translational processing.

In some cases, nucleic acid molecules may comprise a naturally occurring or an artificial promoter. In some cases, the nucleic acid molecule comprises a promoter that is specific for expression in immune cells as compared to non-immune cells. In some cases, the nucleic acid comprises a T-cell specific promoter and the LNPs comprising the nucleic acid can be used for CAR-T therapy. In some cases, the T-cell specific promoter comprises promoters that drive endogenous expression of T-cell specific proteins comprising CD3 (e.g., CD3 gamma, CD3 delta, CD3 epsilon, CD3 zeta), CD4, CD8, CD28, TCRB or TRAC. In some cases, the promoter can result in stronger expression of therapeutic peptides in immune cells comprising lymphocytes, T cells, CD4+ T cells, CD8+ T cells, alpha-beta T cells, gamma-delta T cells, T regulatory cells (Tregs), cytotoxic T lymphocytes, Th1 cells, Th2 cells, Th17 cells, Th9 cells, naïve T cells, memory T cells, effector T cells, effector-memory T cells (TEM), central memory T cells (TCM), resident memory T cells (TRM), follicular helper T cells (TFH), Natural killer T cells (NKTs), tumor-infiltrating lymphocytes (TILs), Natural killer cells (NKs), Innate Lymphoid Cells (ILCs), ILC1 cells, ILC2 cells, ILC3 cells, lymphoid tissue inducer (LTi) cells, B cells, B1 cells, B1a cells, Bib cells, B2 cells, plasma cells, B regulatory cells, memory B cells, marginal zone B cells, follicular B cells, germinal center B cells, antigen presenting cells (APCs), monocytes, macrophages, M1 macrophages, M2 macrophages, tissue-associated macrophages, dendritic cells, plasmacytoid dendritic cells, neutrophils, mast cells, basophils, eosinophils, common myeloid progenitors, common lymphoid progenitors, or any combination thereof.

In some cases, the nucleic acid molecule comprises a hepatocyte-specific promoter. In some cases, the nucleic acid molecule comprises a strong promoter that enhances the expression of the therapeutic peptide for immunotherapy. The strong promoter can comprise a Human cytomegalovirus (hCMV), chicken beta-actin/CMV enhancer (CAG), elongation factor-1alpha (EF1α), or phosphoglycerokinase (PGK) promoter.

In some cases, the nucleic acid molecule may comprise natural, synthetic, and/or artificial nucleotide analogues or bases. In some cases, the synthetic or artificial nucleotide analogues or bases comprise modifications at one or more of a deoxyribose moiety, ribose moiety, phosphate moiety, nucleoside moiety, or a combination thereof.

In some cases, a nucleotide analogue or artificial nucleotide base comprises a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Illustrative alkyl moiety includes, but are not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some cases, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety.

In some cases, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some cases, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C-,4′-C-oxy-methylene-linked bicyclic ribonucleotide monomer.

In some cases, additional modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some cases, a nucleotide analogue comprises a modified base, for example, N1-methylpseudouridine, 5-propynyluridine, 5-propynylcytidine, 6-methyladenine, 6-methylguanine, N, N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes universal bases. By way of example, universal bases include but are not limited to 3-nitropyrrole, 5-nitroindole, or nebularine.

In some cases, one or more modifications optionally occur at the internucleotide linkage. In some instances, a modified internucleotide linkages can include, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates of 3′-5′linkage or 2′-5′linkage; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some cases, one or more modifications comprise a modified phosphate backbone in which the modification generates a neutral or uncharged backbone. In some instances, the phosphate backbone is modified by alkylation to generate an uncharged or neutral phosphate backbone. As used herein, alkylation includes methylation, ethylation, and propylation. In some cases, an alkyl group, as used herein in the context of alkylation, refers to a linear or branched saturated hydrocarbon group containing from 1 to 6 carbon atoms. In some instances, exemplary alkyl groups include, but are not limited to, methyl, ethyl, n-propyl, isopropyl, n-butyl, isobutyl, sec-butyl, tert-butyl, n-pentyl, isopentyl, neopentyl, hexyl, isohexyl, 1, 1-dimethylbutyl, 2,2-dimethylbutyl, 3.3-dimethylbutyl, and 2-ethylbutyl groups.

In some cases, a modified phosphate is a phosphate group as described in U.S. Pat. No. 9,481,905.

In some embodiments, additional modified phosphate backbones comprise methylphosphonate, ethylphosphonate, methylthiophosphonate, or methoxyphosphonate. In some cases, the modified phosphate is methylphosphonate. In some cases, the modified phosphate is ethylphosphonate. In some cases, the modified phosphate is methylthiophosphonate. In some cases, the modified phosphate is methoxyphosphonate.

In some cases, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage.

In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

### Methods

Provided herein are methods of treating a disease or disorder using the compounds or the lipid nanoparticle (LNP) compositions described herein. In some embodiments, the methods comprise using the LNPs to deliver a therapeutic payload for immunotherapy. In some embodiments, the methods comprise using the LNPs to deliver a therapeutic payload to treat a cancer.

In some embodiments, the compounds or lipid compositions (or pharmaceutical compositions) of the present application can be administrated through any suitable routes comprising parenteral delivery (e.g., injections), such as intravenous, intramuscular, subcutaneous, intramedullary injections, as well as intrathecal, direct intraventricular, intraperitoneal, intranasal, or intraocular injections.

In some embodiments, the compounds or lipid compositions can be administered in a local rather than systemic manner, for example, via injection of the pharmaceutical composition directly into a targeted tissue, preferably in a sustained release formulation. Local delivery can be affected in various ways, depending on the tissue to be targeted.

In some embodiments, the compounds or lipid compositions can be injected into the site of injury, disease manifestation, or pain, for example. In some embodiments, the compounds or lipid compositions can be provided in lozenges for oral, tracheal, or esophageal application. In some embodiments, the compounds or lipid compositions can be supplied in liquid, tablet or capsule form for administration to the stomach or intestines. In some embodiments, the compounds or lipid compositions can be supplied in suppository form for rectal or vaginal application. In some embodiments, the compounds or lipid compositions can even be delivered to the eye by use of creams, drops, or even injection.

In some embodiments, provided herein is a method for potent delivery to a cell of a subject comprising administrating to the subject the pharmaceutical composition as described in the present application. In some embodiments, the pharmaceutical composition comprises a therapeutic payload (e.g., pharmaceutical or therapeutic agent) assembled with a lipid composition as described in the present application, wherein the lipid composition comprises an ionizable lipid. The lipid composition may further comprise a steroid, a helper lipid, and/or a polymer conjugated lipid.

In some aspects, the methods provided herein can achieve targeted delivery of the therapeutic payload/agent to a target organ or cell. In some embodiments, the target organ comprises the liver, lung, brain, heart, kidneys, or secondary lymphoid tissues (e.g., spleen or lymph nodes). In some embodiments, the target organ comprises the liver. In some embodiments, the target organ comprises the secondary lymphoid tissues. In some embodiments, the target organ comprises the spleen. Target cells provided herein may comprise a cell of the target organ. In some embodiments, the target cell comprises a cell of the liver. A cell of the liver may comprise a hepatocyte, a Kupffer cell, a Stellate cell, a sinusoidal endothelial cell, or a cholangiocyte. In some embodiments, the target cell comprises a neuron or a glial cell. In some embodiments, the target cell comprises an immune cell. In some embodiments, the target cell comprises a lymphocyte (e.g., T cell or B cell).

In some embodiments, the method comprises delivering a therapeutic payload/agent to a target organ or cell of a subject, thereby providing an effective amount or activity of the therapeutic payload/agent in the target organ or cell that is at least 1.1-fold greater than the amount or activity achieved in a non-target organ or cell of the subject. In some embodiments, the effective amount or activity of the therapeutic agent in the target organ or target cell is at least 1.1-fold greater, at least 1.2-fold greater, at least 1.3-fold greater, at least 1.4-fold greater, at least 1.5-fold greater, at least 1.6-fold greater, at least 1.7-fold greater, at least 1.8-fold greater, at least 1.9-fold greater, at least 2-fold greater, at least 2.1-fold greater, at least 2.2-fold greater, at least 2.3-fold greater, at least 2.4-fold greater, at least 2.5-fold greater, at least 3-fold greater, at least 3.2-fold greater, at least 3.5-fold greater, at least 3.7-fold greater, at least 4-fold greater, at least 4.2-fold greater, at least 4.5-fold greater, at least 4.7-fold greater, at least 5-fold greater, at least 5.2-fold greater, at least 5.5-fold greater, at least 5.7-fold greater, at least 6-fold greater, at least 6.2-fold greater, at least 6.5-fold greater, at least 6.7-fold greater, at least 7-fold greater, at least 7.5-fold greater, at least 8-fold greater, at least 8.5-fold greater, at least 9-fold greater, at least 9.5-fold greater, at least 10-fold greater, at least 10.5-fold greater, at least 11-fold greater, at least 11.5-fold greater, at least 12-fold greater, at least 12.5-fold greater, at least 13-fold greater, at least 13.5-fold greater, at least 14-fold greater, at least 14.5-fold greater, at least 15-fold greater, at least 16-fold greater, at least 17-fold greater, at least 18-fold greater, at least 19-fold greater, at least 20-fold greater, at least 25-fold greater, at least 30-fold greater, at least 35-fold greater, at least 40-fold greater, at least 45-fold greater, at least 50-fold greater, at least 55-fold greater, at least 60-fold greater, at least 65-fold greater, at least 70-fold greater, at least 75-fold greater, at least 80-fold greater, at least 85-fold greater, at least 90-fold greater, at least 95-fold greater, at least 100-fold greater, at least 150-fold greater, at least 200-fold greater, at least 250-fold greater, or at least 300-fold greater, than the amount or activity achieved in a non-target organ or cell of the subject.

In some embodiments, the methods comprise administering the lipid compositions provided herein, thereby providing an effective amount or activity of the therapeutic agent at least 1.1-fold greater than the amount or activity achieved by administering other compositions. In some embodiments, the effective amount or activity of the therapeutic agent results from administering a lipid composition described herein is at least 1.1-fold greater, at least 1.2-fold greater, at least 1.3-fold greater, at least 1.4-fold greater, at least 1.5-fold greater, at least 1.6-fold greater, at least 1.7-fold greater, at least 1.8-fold greater, at least 1.9-fold greater, at least 2-fold greater, at least 2.1-fold greater, at least 2.2-fold greater, at least 2.3-fold greater, at least 2.4-fold greater, at least 2.5-fold greater, at least 3-fold greater, at least 3.2-fold greater, at least 3.5-fold greater, at least 3.7-fold greater, at least 4-fold greater, at least 4.2-fold greater, at least 4.5-fold greater, at least 4.7-fold greater, at least 5-fold greater, at least 5.2-fold greater, at least 5.5-fold greater, at least 5.7-fold greater, at least 6-fold greater, at least 6.2-fold greater, at least 6.5-fold greater, at least 6.7-fold greater, at least 7-fold greater, at least 7.5-fold greater, at least 8-fold greater, at least 8.5-fold greater, at least 9-fold greater, at least 9.5-fold greater, at least 10-fold greater, at least 10.5-fold greater, at least 11-fold greater, at least 11.5-fold greater, at least 12-fold greater, at least 12.5-fold greater, at least 13-fold greater, at least 13.5-fold greater, at least 14-fold greater, at least 14.5-fold greater, at least 15-fold greater, at least 16-fold greater, at least 17-fold greater, at least 18-fold greater, at least 19-fold greater, at least 20-fold greater, at least 25-fold greater, at least 30-fold greater, at least 35-fold greater, at least 40-fold greater, at least 45-fold greater, at least 50-fold greater, at least 55-fold greater, at least 60-fold greater, at least 65-fold greater, at least 70-fold greater, at least 75-fold greater, at least 80-fold greater, at least 85-fold greater, at least 90-fold greater, at least 95-fold greater, at least 100-fold greater, at least 150-fold greater, at least 200-fold greater, at least 250-fold greater, or at least 300-fold greater, than the amount or activity achieved by administering other compositions.

In some embodiments, the delivery of the therapeutic payload or pharmaceutical agent to a cell may alter the genome, transcriptome, or expression levels. The cell may be allowed to, or able to, propagate and the alteration may be passed on to the cells generated from the cell that the therapeutic was delivered to. In this manner, the therapeutic effect may be propagated to a larger number of cells. The alteration to the genome, transcriptome or expression level may also persist in a given cell.

### Dosing Level

In another aspect, provided is high-potency dosage form of a pharmaceutical agent (or a therapeutic agent) formulated with an ionizable lipid, the dosage form comprising a therapeutic agent (e.g., a therapeutic peptide or a nucleic acid encoding the therapeutic peptide) assembled with a lipid composition as described herein.

In some embodiments, the therapeutic agent is present in the dosage form at a dose of about 10, 9, 8, 7, 6, 5, 4, 3, 2, 1.5, 1.0, 0.5, 0.2, 0.1, 0.05, 0.02, 0.01, 0.005, 0.002, or 0.001 milligram per kilogram (mg/kg, or mpk) body weight, or of a range between (inclusive) any two of the foregoing values.

In some embodiments, the therapeutic agent is present in the dosage form at a dose of no more than about 10 milligram per kilogram (mg/kg, or mpk) body weight. In some embodiments, the therapeutic agent is present in the dosage form at a dose of no more than about 9 mg/kg, no more than about 8 mg/kg, no more than about 7 mg/kg, no more than about 6 mg/kg, no more than about 5 mg/kg, no more than about 4 mg/kg, no more than about 3 mg/kg, no more than about 2 mg/kg, no more than about 1 mg/kg, no more than about 0.5 mg/kg, no more than about 0.2 mg/kg, no more than about 0.1 mg/kg, no more than about 0.05 mg/kg, or no more than about 0.01 mg/kg. In some embodiments, the therapeutic agent is present in the dosage form at a concentration of no more than about 5 milligram per milliliter (mg/mL).

In some embodiments, the therapeutic agent is present in the dosage form at a concentration of about 5, 4, 3, 2, 1, 0.5, 0.2, or 0.1 milligram per milliliter (mg/mL), or of a range between (inclusive) any two of the foregoing values.

In some embodiments, the therapeutic agent is present in the dosage form at a concentration of no more than about 5 milligram per milliliter (mg/mL). In some embodiments, the therapeutic agent is present in the dosage form at a concentration of no more than about 2 milligram per milliliter (mg/mL). In some embodiments, the therapeutic agent is present in the dosage form at a concentration of no more than about 1 milligram per milliliter (mg/mL). In some embodiments, the therapeutic agent is present in the dosage form at a concentration of no more than about 0.5 milligram per milliliter (mg/mL). In some embodiments, the therapeutic agent is present in the dosage form at a concentration of no more than about 0.1 milligram per milliliter (mg/mL).

In some embodiments, the therapeutic agent (e.g., proteins, nucleic acids) is present in the dosage form at a concentration of about 10, 9, 8, 7, 6, 5, 4, 3, 2, 1, 0.5, 0.2, or 0.1 microgram per milliliter (g/mL), or of a range between (inclusive) any two of the foregoing values. In some embodiments, the therapeutic agent is present in the dosage form at a concentration of no more than about 10, no more than about 9, no more than about 8, no more than about 7, no more than about 6, no more than about 5, no more than about 4, no more than about 3, no more than about 2, no more than about 1, no more than about 0.5, no more than about 0.2, no more than about 0.1 microgram per milliliter (g/mL).

Any suitable dosage form can be prepared for delivery, for example, via oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments, the pharmaceutical agent is administered at a dosage of no more than about 10 milligram per kilogram (mg/kg, or mpk) body weight, no more than about 9 mg/kg, no more than about 8 mg/kg, no more than about 7 mg/kg, no more than about 6 mg/kg, no more than about 5 mg/kg, no more than about 4 mg/kg, no more than about 3 mg/kg, no more than about 2 mg/kg, no more than about 1 mg/kg, no more than about 0.5 mg/kg, no more than about 0.2 mg/kg, no more than about 0.1 mg/kg, no more than about 0.05 mg/kg, or no more than about 0.01 mg/kg body weight. In some embodiments, the pharmaceutical agent is administered at a dosage from about 1 μg/kg body weight to about 3 mg/kg body weight.

In some embodiments, the administration of a dose of the lipid composition provided here can be repeated. If desired, the effective dose of the active lipid composition can be administered as one, two, three, four, five, six or more doses administered separately at appropriate intervals throughout the course of treatment. In some embodiments, the lipid composition can be administered two or three times daily. In some embodiments, the lipid composition will be administered once daily. In some embodiments, the lipid composition is administered about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, or about every 18 weeks. In some embodiments, the lipid composition is administered about every 1 month, about every 2 months, about every 3 months, about every 4 months, about every 5 months, about every 6 months, about every 7 months, about every 8 months, about every 9 months, about every 10 months, about every 11 months, about every 12 months, about every 13 months, about every 14 months, about every 15 months, about every 16 months, about every 17 months, about every 18 months, about every 2 years, about every 2.5 years, about every 3 years, about every 3.5 years, about every 4 years, about every 4.5 years, or about every 5 years. Any subject in need thereof can be treated with the method of the present application. In some embodiments, the subject has been determined to likely respond to the therapeutic agent. For example, the subject may have, is suffering from, or suspected of having a disease or condition. The therapeutic or prophylactic agent(s) as described elsewhere herein may be effective for providing a therapeutic effect for the subject by a variety of mechanisms, for example, via gene therapy (e.g., requiring repeated administration), altered protein production, (e.g., in vivo) chimeric antigen receptor (CAR) T-cell generation, immuno-oncology, vaccine-based approach, reactivation of tumor suppressors, or other mechanisms.

In some embodiments, the subject has been determined to have a (e.g., missense or nonsense) mutation in a target gene. In some embodiments, the mutation in the target gene is associated with a genetic disease or disorder.

In some embodiments, the subject has been determined to exhibit an aberrant expression or activity of a protein or polynucleotide that corresponds to a target gene. In some embodiments, the aberrant expression or activity of the protein or polynucleotide is associated with a genetic disease or disorder.

In some embodiments, the subject is selected from the group consisting of mouse, rat, monkey, and human. In some embodiments, the subject is a human.

In some embodiments, provided herein is a method for targeted delivery of a therapeutic agent to a cell type comprising contacting the cell with the pharmaceutical composition of the present application. In some embodiments of the method, the pharmaceutical composition comprises a therapeutic agent assembled with a lipid composition as described in the present application, e.g., wherein the lipid composition comprises any of the head or tail groups disclosed herein.

In some embodiments, the contacting is ex vivo. In some embodiments, the contacting is in vitro. In some embodiments, the contacting is in vivo. In some embodiments, the contacting comprises administering to a subject the composition comprising the therapeutic agent assembled with the lipid composition.

### Pharmaceutical Compositions

The compositions and methods of the present disclosure may be utilized to treat an individual in need thereof. The pharmaceutical composition described herein may comprise a therapeutic or prophylactic composition, or any combination thereof. In some embodiments, the lipidoid compositions can be assembled with mRNA encoding a therapeutic peptide (e.g., antigen-binding peptide or immunomodulator) for immunotherapy. In some embodiments, the individual is a mammal (e.g., a human or a non-human mammal). When administered to an animal (e.g., a human or a non-human animal) the composition or the lipidoid composition is preferably administered as a pharmaceutical composition comprising, for example, a lipidoid composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as intravenous or intramuscular injection, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a lipidoid composition such as a lipidoid composition of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipidoid composition of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipidoid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipidoid composition may also be formulated for inhalation. In some embodiments, a lipidoid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipidoid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipidoid (e.g., nanoparticle) composition as described herein, with the carrier and, optionally, one or more accessory ingredients. In general, the formulations are prepared by uniformly and intimately bringing into association a lipidoid (e.g., nanoparticle) composition as described herein with liquid carriers, or finely divided solid carriers, or both, and then, if necessary, shaping the product.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. In some embodiments, the pharmaceutical compositions comprising the mRNA/LNPs provided herein are administered through parenteral routes (e.g., intravenous injection or intramuscular injection). Pharmaceutical compositions suitable for parenteral administration comprise one or more active lipidoid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject lipidoid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active lipidoid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a lipidoid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipidoid composition or combination of lipidoid compositions employed, or the ester, salt or amide thereof, the route of administration, the time of administration, the rate of excretion of the particular lipidoid composition(s) being employed, the duration of the treatment, other drugs, lipidoid compositions and/or materials used in combination with the particular lipidoid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipidoid composition at levels lower than that required in order to achieve the desired therapeutic effect and gradually increase the dosage until the desired effect is achieved. By “therapeutically effective amount” is meant the concentration of a lipidoid composition that is sufficient to elicit the desired therapeutic effect. It is generally understood that the effective amount of the lipidoid composition will vary according to the weight, sex, age, and medical history of the subject. Other factors which influence the effective amount may include, but are not limited to, the severity of the patient's condition, the disorder being treated, the stability of the lipidoid composition, and, if desired, another type of therapeutic agent being administered with the lipidoid composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipidoid composition used in the compositions and methods of the invention will be that amount of the lipidoid composition that is the lowest dose effective to produce a therapeutic effect. Such an effective dose will generally depend upon the factors described above.

If desired, the effective dose of the active lipidoid composition may be administered as one, two, three, four, five, six or more doses administered separately at appropriate intervals throughout the course of treatment, optionally, in unit dosage forms. In some embodiments of the present invention, the active lipidoid composition may be administered two or three times daily. In some embodiments, the active lipidoid composition will be administered once daily.

The patient or subject receiving this treatment is any animal in need, including primates, in particular humans; and other mammals such as equines, cattle, swine, sheep, cats, and dogs; poultry; and pets in general.

In some embodiments, lipidoid compositions of the invention may be used alone or conjointly administered with another type of therapeutic agent.

The present disclosure includes the use of pharmaceutically acceptable salts of lipidoid compositions of the invention in the compositions and methods of the present invention. In some embodiments, contemplated salts of the invention include, but are not limited to, alkyl, dialkyl, trialkyl or tetra-alkyl ammonium salts. In some embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In some embodiments, contemplated salts of the invention include, but are not limited to, Na, Ca, K, Mg, Zn or other metal salts. In some embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d glucoheptonic acid, d gluconic acid, d glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1 tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Kits

Another aspect provides a kit comprising the pharmaceutical composition and/or lipid nanoparticle (LNP) formulations described herein. In some embodiments, the kit can further comprise an apparatus for administering the compositions provided herein, such as appropriate syringes for intravenous or intramuscular administration.

In any herein-disclosed method, the method further comprises providing instructions for use (IFU), the IFU including instructions for administering the LNP compositions to a subject. In some embodiments, the user instruction directs a user to inject the LNP compositions comprising mRNA intravenously or intramuscularly for use in immunotherapy.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Chemical Synthesis

### Lipid Synthesis

Lipids synthesized are listed in TABLE 2. Homogeneous lipids (e.g., lipids comprising one kind of lipid tails) were synthesized through a solvent free Michael Addition reaction between an amine head group and an alkyl-acrylate lipid tail. A headgroup comprising an amine, for example, the aliphatic headgroup 3,3′-Diamino-N-methyldipropylamine, and an acrylate tail were mixed at 1 to 5 molar ratio in Teflon-lined glass screw-top vials at 70° C. for 48 h. The crude products were purified using a Teledyne Isco Chromatography system using methanol/DCM as mobile phase. The purified lipidoids were characterized by electrospray ionization mass spectrometry (ESI-MS).

In the cases of asymmetric heterogenous lipids (e.g., lipids comprising different lipid tails), the headgroup comprising an amine was mixed with a first kind of lipid tail at 1 to 3.5 molar ratio. After 48 hours of stirring at 70° C., the partially assembled lipid products were purified as described above. Subsequently the partially assembled lipid products were mixed with a second lipid tail at 1: 1.5 molar ratio. The final asymmetric heterogenous lipids were isolated and verified by ESI-MS.

In the cases of symmetric heterogenous lipids (e.g., lipids comprising equal numbers of different lipid tails), headgroups comprising Boc-protected amines were reacted with a first kind of lipid tail at 1 to 2.5 molar ratio. After mixing at 70° C. for 48 hours, the half-assembled lipid products were purified. The Boc protecting group was removed by trifluoroacetic acid in DCM, and the half-assembled lipids were further reacted with a second lipid tail to afford the full heterogenous but symmetric lipid products.

### Lipid Tail Synthesis

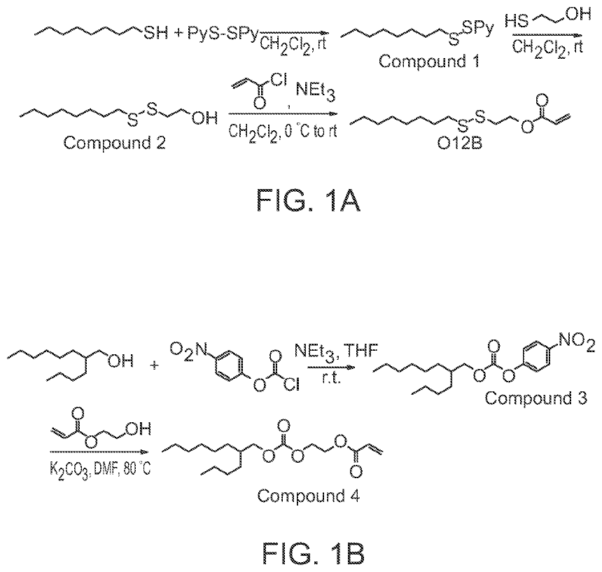

The synthesis of lipid tail 012B is illustrated in FIG. 1A as an example for lipid tail synthesis. Briefly, 2,2′-dipyridyl disulfide was dissolved in dichloromethane under inert gas in presence of trace amount of acetic acid. 1-octanethiol solution in dichloromethane was then added dropwise. The reaction mixture was stirred overnight and concentrated by rotary evaporation. Crude reaction mixture was filtered through a short pad of silica gel and flushed with ethyl acetate/hexanes mixture. The collected solvents were concentrated, and the residue was purified by flash column chromatography on silica to obtain Compound 1 in FIG. 1A with good yield. Compound 2 was prepared and purified in a similar way. 2-mercaptoethanol was used as nucleophile for the synthesis of Compound 2 under inert gas. For lipid tail 012B synthesis, Compound 2 was dissolved in dichloromethane under an inert atmosphere and cooled to 0° C. 1.5 equivalent of triethylamine was added while stirring. Then neat acryloyl chloride (1.2 eq.) was added slowly. Crude reaction mixture was concentrated by rotary evaporation, filtered through a short pad of silica gel and flushed with ethyl acetate/hexanes mixture. The collected solvents were concentrated, and the residue was purified by flash column chromatography on silica to obtain lipid tail with satisfactory overall yield.

The synthesis of alkene lipid tail replies on a similar scheme to the above-mentioned, wherein the biodegradable alcohol Compound 2 with disulfide bond is replaced with the commercially available unsaturated alcohol cis-5-octene-1-ol.

The synthesis of Compound 4, a lipid tail comprising carbonate, is illustrated in FIG. 1B. Briefly, 4-Nitrophenyl chloroformate (20.2.6 g, 110 mmol) was added in portions to a solution of 2-butyl-1-octanol (18.6 g, 100 mmol) and triethylamine (27.9 ml, 200 mmol) in THE under Argon in a round flask with a magnetic stir bar at room temperature. The reaction mixture was stirred for 2-3 hours until completion, as monitored by TLC. Then the reaction mixture was diluted with ethyl acetate, and washed sequentially with 1N HCl solution, saturated NaHCO₃ solution, and brine. The organic layers were dried over MgSO₄, filtered, and concentrated. The residue was purified by flash column chromatography on silica to afford Compound 3 (31.0 g, 88.3 mmol, 88%).

Compound 3 (5.62 g, 16 mmol) was dissolved in DMF and added to a solution of 2-hydroxyethyl acrylate (1.86 g, 16 mmol) in DMF in a two-neck round-bottom flask charged with a magnetic stir bar under Ar. The reaction mixture was heated to 50° C., and K₂CO₃ (5.5 g, 40 mmol) was added portion wise to the reaction mixture after 10 min. Then the reaction mixture was heated to 80° C. for 2-3 hours until completion. DMF was removed by rotary evaporation and the residue was partitioned by addition of water and ethyl acetate. The aqueous layer was extracted with ethyl acetate three times. The combined organic layers were washed with water and brine, dried over Na₂SO₄, filtered, and concentrated. The residue was purified by flash column chromatography on silica to afford Compound 4 (3.68 g, 11.2 mmol, 70%).

### Example 2: LNP Formulation and Characterization

RNA working solutions (0.134 mg/ml) were prepared in 25 mM sodium acetate buffer using DNase/RNase-free distilled water. Lipid stock solutions (10 mg/mL) were freshly prepared with active lipids or helper lipids (e.g., cholesterol, DOPC or DSPC, or ALC-0159) in ethanol. Reagents were mixed by vortexing and sonicating until the lipids were completely dissolved. A lipid mixture was first prepared by combining the active lipids (e.g., ionizable lipids of the present disclosure) and helper lipids into one vial and mixing thoroughly. Lipid nanoparticles were then formed by mixing the RNA working solution (0.134 mg/ml) and the lipid mixture at 3:1 volume ratio, either manually (for smaller scale) or by Precision Igniter microfluidic device. The formulation solution was transferred into a 300 kDa MWCO Float-A-Lyzer G2 Dialysis Device and dialyzed against at least 1000-folds volume of 50 mM Tris buffer overnight at 4° C. The LNP solution was sterilized with a 0.22 μm filter. TABLE 3 demonstrates an example of a lipid nanoparticle formulation.

TABLE 3
Example calculation for one LNP formulation

| | RNA | Lipid | Weight ratio | | Volume for |
|---|---|---|---|---|---|
| | working | mixtures | among lipid | | each lipid |
| | solution | volume | components | | component |
| Formulation | (uL) | (uL) | (10 mg/mL) | | (μL) |
| LNP-0088 | 1500 | 436 | Active lipid | 16.6 | 332 |
| | | | (HTX-CH- | | |
| | | | L0088) | | |
| | | | Cholesterol | 4 | 80 |
| | | | DOPC | 2 | 40 |
| | | | DMG-PEG | 1 | 20 |

For LNP characterization, size and polydispersity index (PDI) were determined by dynamic light scattering using a Zetasizer Nano ZS. 10 μL of LNP suspension was transferred to a low-volume cuvette containing 900 μL of Ca²⁺ and Mg²⁺ free PBS at pH 7.4. Refractive index was set to 1.14 and temperature was set to 25° C. Encapsulation efficiency (EE %) was measured using a fluorescence plate-based assay employing the Ribogreen reagent (Invitrogen) as per PNI Ribogreen assay protocol 16. This assay measures the quantity of mRNA in samples with intact LNPs to determine the quantity of unencapsulated RNA as well as in LNP samples disrupted by triton X-100 to measure the total RNA. EE % is calculated as the difference between the total RNA and the unencapsulated RNA divided by the total RNA. Formulations and results from characterization assays are listed in TABLE 4.

TABLE 4
Characterization of LNPs with mRNA payload

| | Molar percentage of lipids | | | | | | | | |
|---|---|---|---|---|---|---|---|---|---|
| | Active | | | DMG- | | Size | | | [mRNA] |
| Lipid # | lipid | Cholesterol | DOPC | PEG | Lipids:mRNA | (nm) | PDI | EE % | Ug/ml |
| L0665 | 16.8 | 4.0 | 2.0 | 1.0 | 25:1 | 107.6 | 0.16 | 97.2 | 65.8 |
| L0666 | 16.8 | 4.0 | 2.0 | 1.0 | 25:1 | 246.5 | 0.26 | 96.1 | 71.9 |
| L0667 | 16.8 | 4.0 | 2.0 | 1.0 | 25:1 | 101.7 | 0.19 | 97 | 77.3 |
| L0668 | 16.8 | 4.0 | 2.0 | 1.0 | 25:1 | 134.7 | 0.11 | 97 | 67.1 |
| L0674 | 16.8 | 4.0 | 2.0 | 1.0 | 25:1 | 124.4 | 0.22 | 98.92 | 71.22 |
| L0773 | 16.8 | 4.0 | 2.0 | 1.0 | 25:1 | 135.3 | 0.089 | 98.7 | 95.3 |
| L0824 | 16.8 | 4.0 | 2.0 | 1.0 | 25:1 | 95.98 | 0.12 | 68 | 93.4 |
| L0826 | 16.8 | 4.0 | 2.0 | 1.0 | 25:1 | 141 | 0.082 | 39 | 95.2 |
| L0823 | 16.8 | 4.0 | 2.0 | 1.0 | 25:1 | 117 | 0.069 | 52 | 94 |
| L0586 | 16.8 | 4.0 | 2.0 | 1.0 | 25:1 | 151.8 | 0.079 | 21.2 | 78 |
| L0630 | 16.8 | 4.0 | 2.0 | 1.0 | 25:1 | 121.9 | 0.102 | 60.74 | 74.99 |
| L0631 | 16.8 | 4.0 | 2.0 | 1.0 | 25:1 | 122.2 | 0.105 | 45.59 | 88.88 |
| L0825 | 16.8 | 4.0 | 2.0 | 1.0 | 25:1 | 118.7 | 0.099 | 62 | 91.2 |
| L0550 | 16.8 | 4.0 | 2.0 | 1.0 | 25:1 | 121 | 0.14 | 84.3 | 61.1 |
| L0637 | 16.8 | 4.0 | 2.0 | 1.0 | 25:1 | 186.1 | 0.34 | 94 | 66.8 |
| L0638 | 16.8 | 4.0 | 2.0 | 1.0 | 25:1 | 121.5 | 0.22 | 95.7 | 65.1 |
| L0654 | 16.8 | 4.0 | 2.0 | 1.0 | 25:1 | 119.4 | 0.1 | 90.6 | 65.6 |
| L0659 | 16.8 | 4.0 | 2.0 | 1.0 | 25:1 | 104.3 | 0.1 | 96.2 | 69.6 |
| L0660 | 16.8 | 4.0 | 2.0 | 1.0 | 25:1 | 122.2 | 0.11 | 97 | 71 |
| L0664 | 16.8 | 4.0 | 2.0 | 1.0 | 25:1 | 149.1 | 0.09 | 97.8 | 74.3 |
| L0744 | 16.8 | 4.0 | 2.0 | 1.0 | 25:1 | 97 | 0.118 | 95.5 | 74 |
| L0775 | 16.8 | 4.0 | 2.0 | 1.0 | 25:1 | 5911 | 0.863 | | |
| L0642 | 16.8 | 4.0 | 2.0 | 1.0 | 25:1 | 186.4 | 0.124 | 84.5 | 99.9 |
| L0645 | 16.8 | 4.0 | 2.0 | 1.0 | 25:1 | 161 | 0.106 | 74.2 | 90 |
| L0813 | 16.8 | 4.0 | 2.0 | 1.0 | 25:1 | 183 | 0.107 | 84 | 96.7 |
| L0814 | 16.8 | 4.0 | 2.0 | 1.0 | 25:1 | 178.8 | 0.093 | 70 | 90.2 |
| L0829 | 16.8 | 4.0 | 2.0 | 1.0 | 25:1 | 98.3 | 0.13 | 92 | 78 |
| L0830 | 16.8 | 4.0 | 2.0 | 1.0 | 25:1 | 125.1 | 0.105 | 48 | 91 |
| L0854 | 16.8 | 4.0 | 2.0 | 1.0 | 25:1 | 120.3 | 0.098 | 94.6 | 77.7 |
| L0855 | 16.8 | 4.0 | 2.0 | 1.0 | 25:1 | 99.86 | 0.125 | 95.1 | 78.9 |
| L0088 | 16.8 | 4.0 | 2.0 | 1.0 | 25:1 | 153.9 | 0.087 | 85.2 | 69.7 |

### Example 3: MRNA/LNP Delivery to Mice

IV Injection of Luc mRNA/LNP Resulted in Protein Expression In Vivo

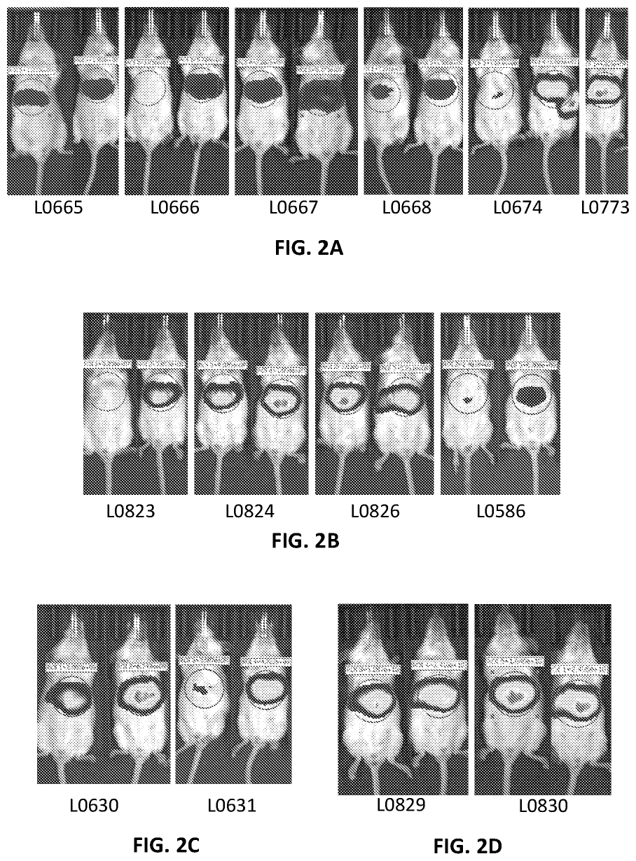

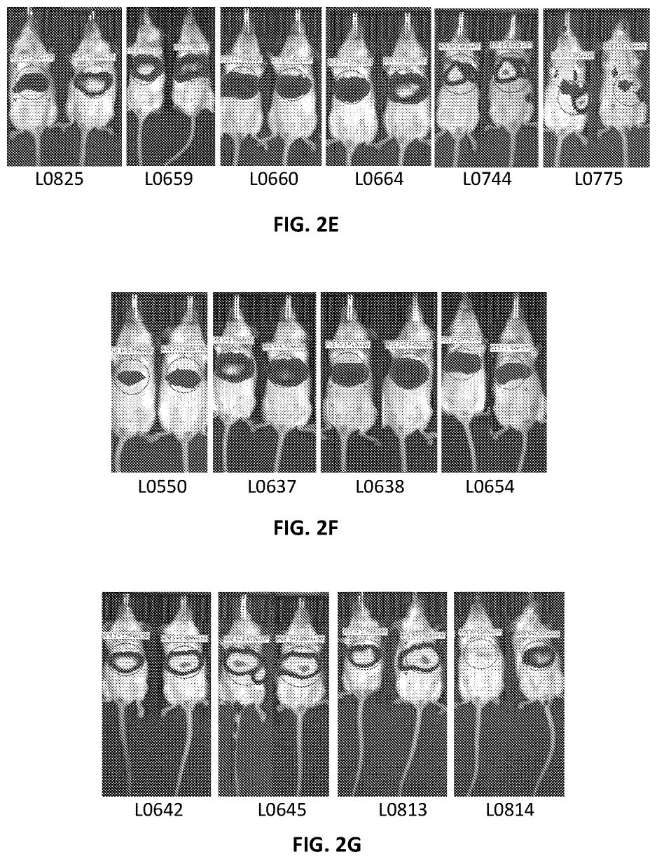

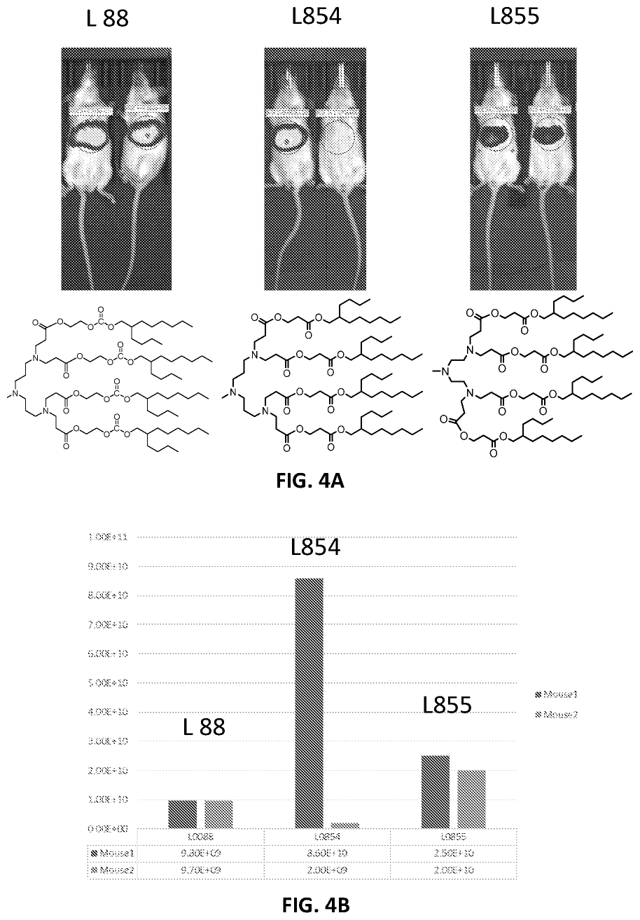

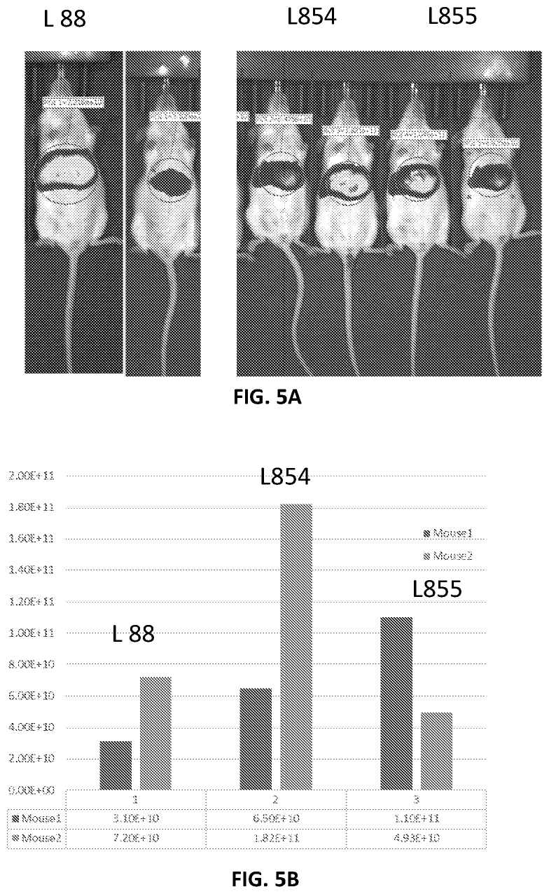

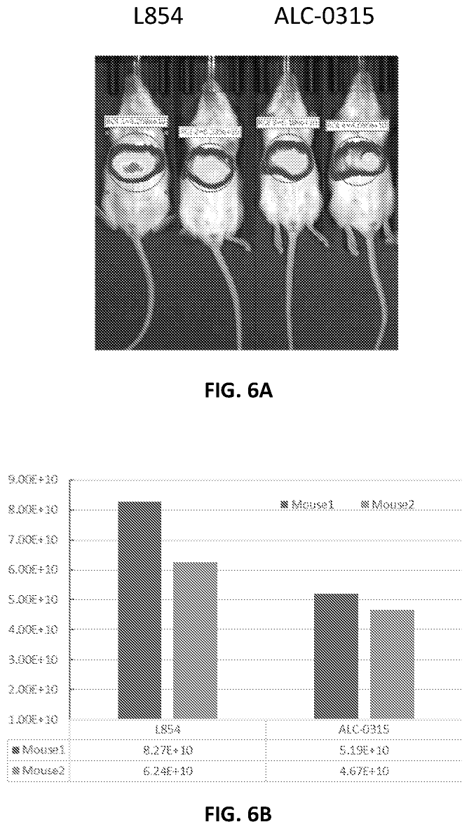

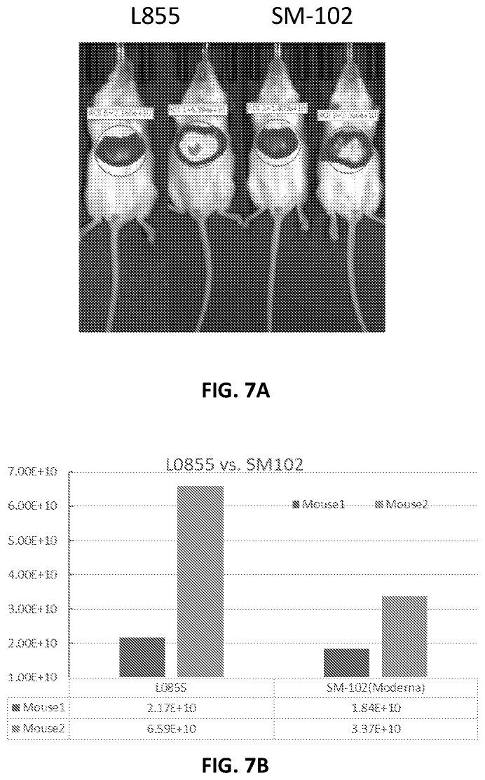

All animal experiments were conducted in accordance with the approved animal protocols. LNPs were formulated with lipids and mRNA encoding Luciferase (Luc) based on TABLE 3. LNPs comprising 5 μg mRNA and 50 μg active lipids (e.g., ionizable lipids) were injected intravenously (i.v.) to female adult BALB/c mice (6-8 wk. old). Six hours post injection, 100 μL of luciferin K salt (15 mg/mL in PBS) was administered to the mice through intraperitoneal injections. The mice were then imaged using the In Vivo Imaging System (IVIS) and the total flux of areas of interest was quantified. FIGS. 2A-2G illustrate representative IVIS data using the LNPs formulated with active lipids from TABLE 4. As depicted in FIGS. 2A-2G, i.v. injections of Luc mRNA/LNPs to the mice resulted in in vivo production of Luc, primarily observed in the liver. FIGS. 4A-7B show further analysis of the IVIS results. In FIGS. 4A-4B and FIGS. 5A-5B, the lipid nanoparticles were formulated with luciferase mRNA and lipids L88, L854, and L855, respectively. Quantification of luciferase intensity is shown in FIG. 4B and FIG. 5B. The experiment was conducted in duplicates. However, due to lab error the second mouse in FIG. 4A-4B intended for testing the luciferase mRNA/L854-LNP did not receive the full injection. Lipid nanoparticles formulated with L854 and L855 resulted in more efficient delivery of the luciferase mRNA to the liver in vivo. Existing lipid formulations in the industry were used as controls in the experiment. For example, lipids provided herein were compared to ALC-0315 (Pfizer) and SM-102 (Moderna). As shown in FIGS. 6A-6B LNPs formulated with lipids L854 resulted in more luciferase mRNA delivery to the liver than ALC-0315. Similarly, as depicted in FIGS. 7A-7B, LNPs formulated with lipids L855 resulted in more luciferase mRNA delivery than SM-102 in vivo.

IM Injection of hEPO mRNA/LNP Resulted in Protein Expression In Vivo

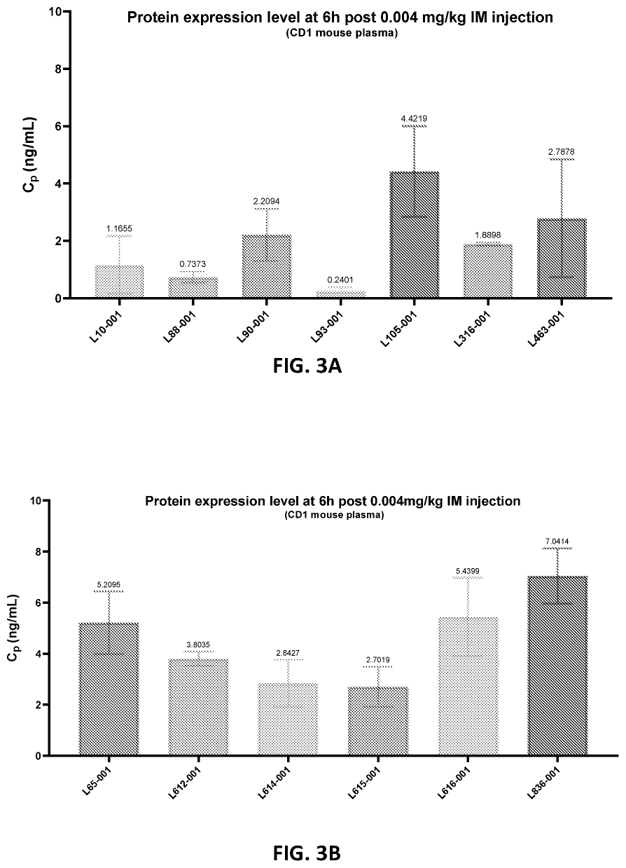

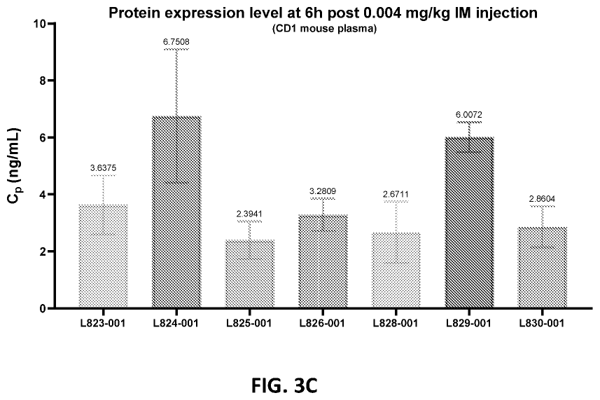

CD-1 mice were administered a single intramuscular (IM) dose of 0.004 mg/kg LNP comprising modified mRNA encoded human erythropoietin (hEPO). Blood was collected from mice 6 hours post injection via the submandibular vein. Samples were centrifuged at 3000×g for 10 minutes and the plasma samples were aliquoted and stored at −80° C. until analysis. hEPO concentration in the plasma was determined via ELISA, and quantification analysis was performed using Softmax Pro (Molecular device). Results were shown in FIGS. 3A-3C as mean±SD. As depicted in FIGS. 3A-3C, hEPO was detected in all samples tested, indicating that the LNP formulations used successfully delivered the modified hEPO mRNA to inside the cells.

1. A compound represented by Formula (III): 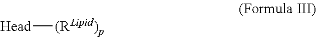
or a pharmaceutically acceptable salt thereof;
wherein:
the Head is selected from the group consisting of 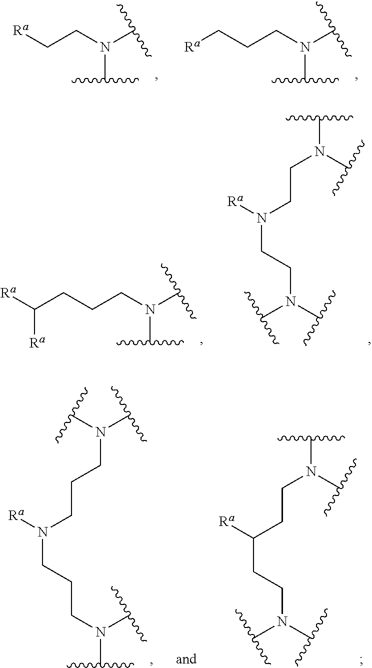
wherein each R^{a} is independently selected from the group consisting of H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₃-C₇ carbocycle, and 3- to 7-membered heterocycle;
each R^{Lipid} is independently selected from the group consisting of: 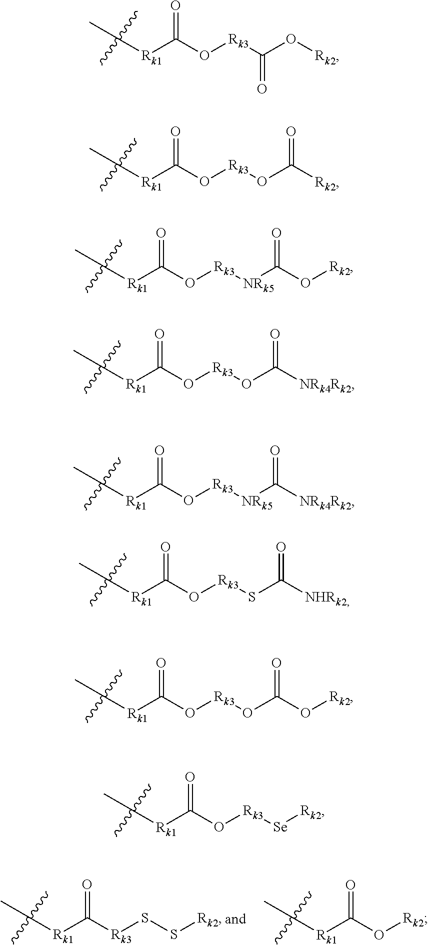
Rₖ₁ and Rₖ₃ are each independently C₁-C₁₀ alkyl;
each Rₖ₂ is independently selected from the group consisting of C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl;
each Rₖ₄ and Rₖ₅ are independently selected from the group consisting of H and C₁-C₁₀ alkyl; and
p is selected from the group consisting of 1, 2, 3, 4, 5, and 6.

2. The compound or pharmaceutically acceptable salt of claim 1, wherein R^{Lipid} is selected from the group consisting of

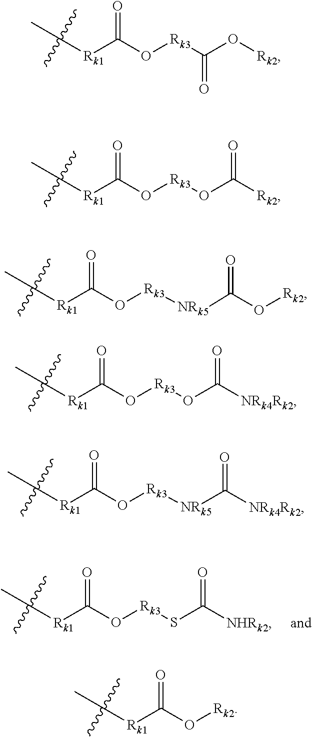

3. The compound or pharmaceutically acceptable salt of claim 1, wherein the Head is selected from the group consisting of

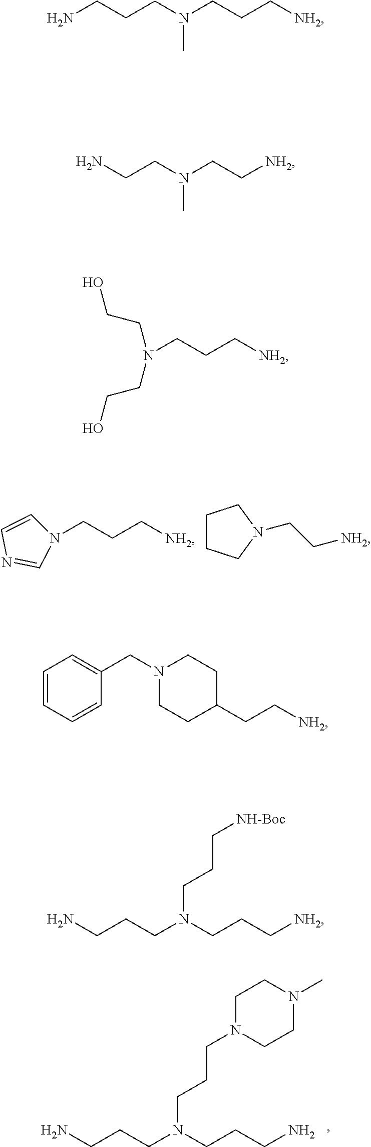

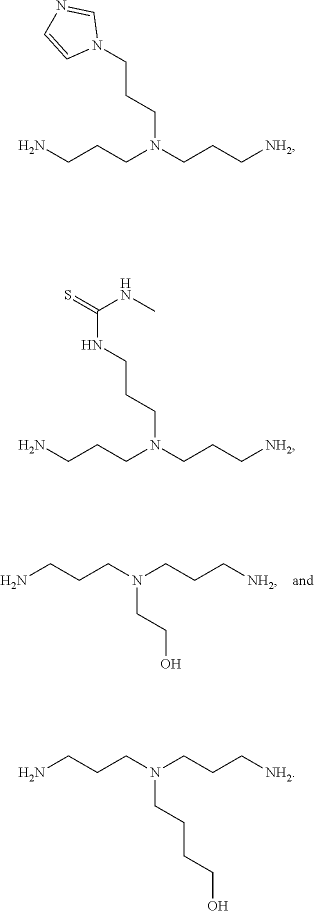 [SMILES: NCCCN(CCCN)CCCn1ccnc1 ; CNC(=S)NCCCN(CCCN)CCCN ; NCCCN(CCO)CCCN ; "and" ; NCCCN(CCCN)CCCCO]

4. The compound or pharmaceutically acceptable salt of claim 1, wherein R^{Lipid} is represented by a structure in TABLE 1.

5. The compound or pharmaceutically acceptable salt of claim 1, wherein the compound is represented by a structure in TABLE 2.

6. A composition comprising a pharmaceutical agent assembled with a lipid composition comprising an ionizable lipid represented by Formula (III): 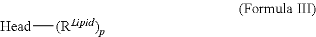
or a pharmaceutically acceptable salt thereof;
wherein:
the Head is selected from the group consisting of 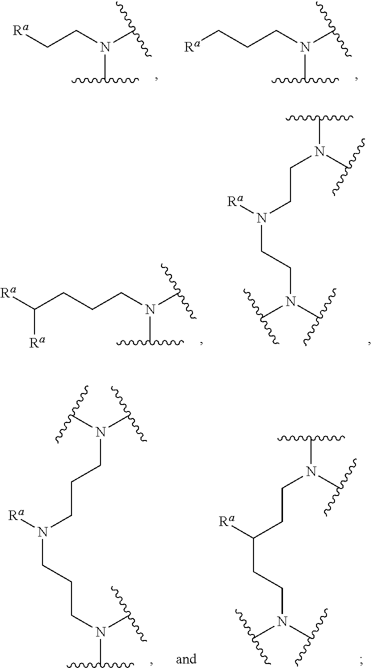
wherein each R^{a} is independently selected from the group consisting of H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₃-C₇ carbocycle, and 3- to 7-membered heterocycle;
each R^{Lipid} is independently selected from the group consisting of: 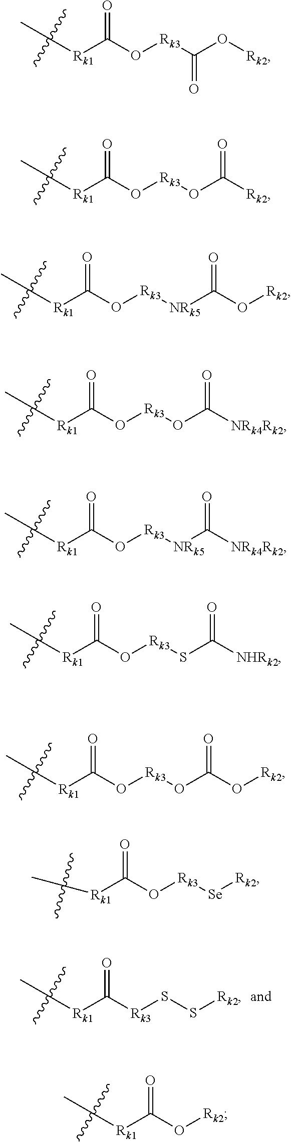
Rₖ₁ and Rₖ₃ are each independently C₁-C₁₀ alkyl;
each Rₖ₂ is independently selected from the group consisting of C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl;
each Rₖ₄ and Rₖs are independently selected from the group consisting of H and C₁-C₁₀ alkyl; and
p is selected from 1, 2, 3, 4, 5, and 6.

7. A composition comprising a pharmaceutical agent assembled with a lipid composition comprising an ionizable lipid, wherein the ionizable lipid has an amine head group and at least one hydrophobic tail R^{Lipid} having a structure of Formula (I): 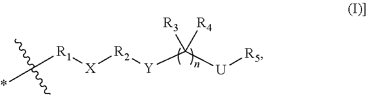
or a pharmaceutically acceptable salt thereof;
wherein:
*indicates a point of attachment to a nitrogen in the amine headgroup;
R₁ and R₂ are each independently selected from the group consisting of C₁-C₁₂ alkylene, C₁-C₁₂ alkenylene, and 1- to 12-membered heteroalkylene;
X is 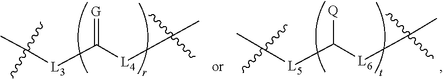
each of L₃, L₄, L₅, and L₆ is independently selected from the group consisting of a bond, O, S, and NR^{c};
G is selected from the group consisting of O, S, and NR^{d};
Q is selected from the group consisting of OR^{e}, SR^{f}, and NR^{g}R^{h};
r is selected from the group consisting of 1, 2, 3, 4, 5, and 6;
t is selected from the group consisting of 1, 2, 3, 4, 5, and 6;
each of R^{e}, R^{d}, R^{c}, R^{f}, R^{g}, and R^{h} is independently selected from the group consisting of H, C₁-C₁₀ alkyl, 1- to 10-membered heteroalkyl, aryl, and heteroaryl;
Y and U are each independently selected from the group consisting of a bond, O, S, NR¹⁰, and Se;
n is 0 or 1;
R₃ and R₄, are each independently selected from the group consisting of H, C₁-C₁₀ alkyl, 1- to 10-membered heteroalkyl, aryl, and heteroaryl;
or R₃ and R₄ together with the atom to which they are attached to form ═O;
R₅ is selected from the group consisting of C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl;
R¹⁰ is selected from the group consisting of H and C₁-C₅ alkyl; and
the pharmaceutical agent comprises an mRNA encoding at least one therapeutic peptide for immunotherapy.

8. The composition of claim 7, wherein the at least one hydrophobic tail R^{Lipid} has a structure selected from the group consisting of 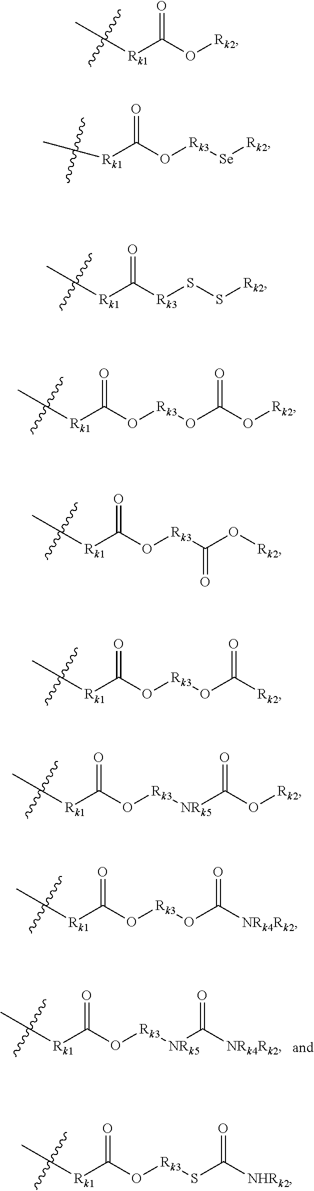
wherein:
each Rₖ₁ and Rₖ₃ is independently C₁-C₁₀ alkyl;
each Rₖ₂ is independently selected from the group consisting of C₁-C₂₀ alkyl, C₂-C₂₀ alkenyl, C₂-C₂₀ alkynyl, C₃-C₂₀ cycloalkyl, 1- to 20-membered heteroalkyl, 3- to 20-membered heterocycloalkyl, aryl, and heteroaryl; and
each Rₖ₄ and Rₖ₅ is independently selected from the group consisting of H and C₁-C₁₀ alkyl.

9. The composition of claim 7, wherein the amine head group is represented by: 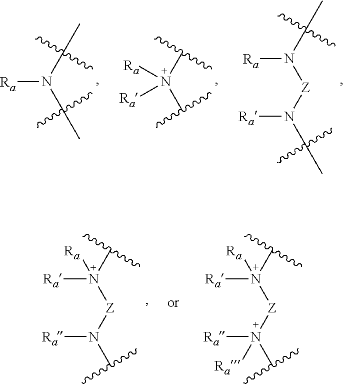
wherein Ra, Ra′, Ra″, and R^{a}′″ are each independently selected from the group consisting of H, C₁-C₂₀ alkyl, C₂-C₂₀ alkenyl, C₂-C₂₀ alkynyl, C₃-C₂₀ cycloalkyl, 3- to 20-membered heterocycloalkyl, 1- to 20-membered heteroalkyl, C₆-C₂₀ aryl, and 6- to 20-membered heteroaryl; and
Z is selected from the group consisting of C₁-C₂₀ alkylene, a 1- to 20-membered heteroalkylene, arylene, and heteroarylene.

10. The composition of claim 7, wherein the amine headgroup is represented by Formula (II): 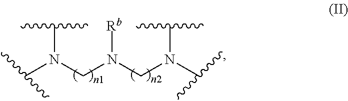
or a pharmaceutically acceptable salt thereof, wherein:
R^{b} is substituted or unsubstituted C₁-C₆ alkyl; and
n1 and n2 are each independently selected from the group consisting of 1, 2, 3, 4, 5, and 6.

11. The composition of claim 7, wherein the amine head group is selected from the group consisting of

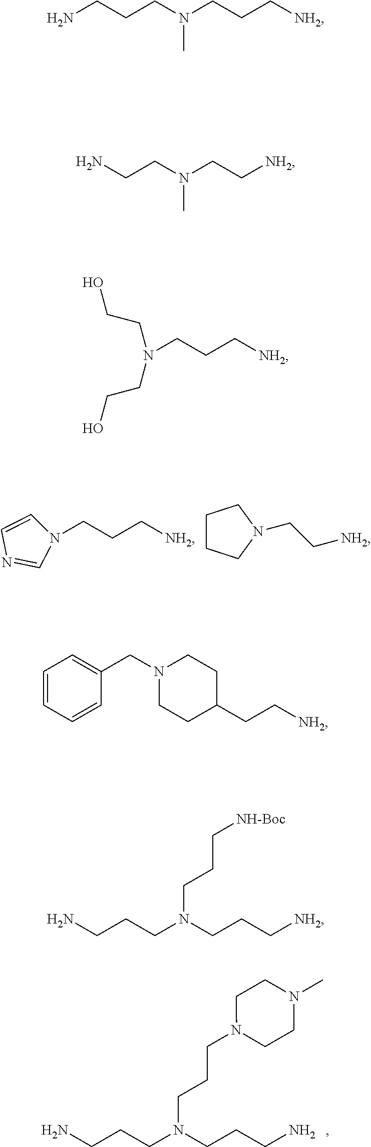

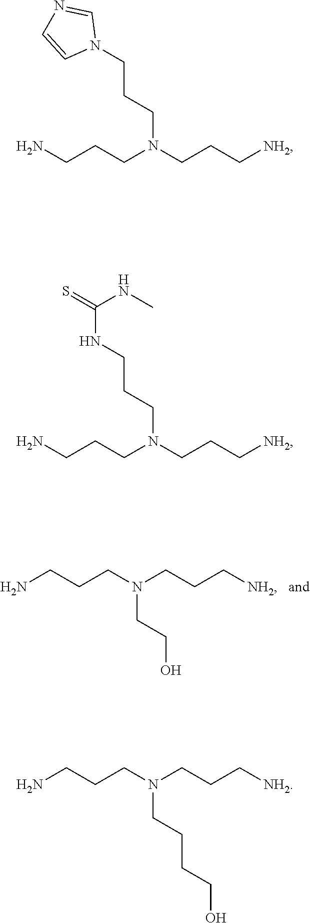 [SMILES: NCCCN(CCCN)CCCn1ccnc1 ; CNC(=S)NCCCN(CCCN)CCCN ; NCCCN(CCO)CCCN ; "and" ; NCCCN(CCCN)CCCCO]

12. The composition of claim 6, wherein the lipid composition further comprises a steroid.

13. (canceled)

14. The composition of claim 6, wherein the lipid composition further comprises a helper lipid.

15. (canceled)

16. The composition of claim 6, wherein the lipid composition further comprises a polymer conjugated lipid.

17. The composition of claim 16, wherein the polymer conjugated lipid is a PEG conjugated lipid.

18. (canceled)

19. (canceled)

20. The composition of claim 16, wherein the lipid composition further comprises a steroid, a helper lipid, and a polymer conjugated lipid.

21-27. (canceled)

28. The composition of claim 6, wherein the lipid composition further comprises a steroid and a helper lipid.

29-32. (canceled)

33. The composition of claim 6, wherein the composition further comprises an excipient.

34. The composition of claim 33, wherein the excipient is selected from the group consisting of (2-hydroxypropyl)-β-cyclodextrin ((HP-β-CD), stearic acid, Perfluoroundecanoic, Saponin, Mannitol, Borneol, Amikacin-EC16, Kanamycin-EC16, Neomycin-EC16, 80-EC16, and Bile salts.

35-41. (canceled)

42. A method for delivering a pharmaceutical agent to a target organ in a subject in need thereof, the method comprising administering an effective amount of the composition according to claim 6.

43-50. (canceled)
